(19) United States
(12) Patent Application Publication
(10) Pub. No.: US 2022/0338750 A1
(43) Pub. Date: Oct. 27, 2022

(54) DEVICES, SYSTEMS, AND METHODS FOR MAGNETIC RESONANCE IMAGING (MRI)-GUIDED PROCEDURES

(71) Applicants: UNIVERSITY OF VIRGINIA PATENT FOUNDATION, Charlottesville, VA (US); VIRGINIA TECH INTELLECTUAL PROPERTIES, INC., Blacksburg, VA (US)

(72) Inventors: Steven P. ALLEN, Charlottesville, VA (US); Craig H. Meyer, Charlottesville, VA (US); Eli VLAISAVLJEVICH, Blacksburg, VA (US); Richey M. DAVIS, Blacksburg, VA (US); Austin D. FERGUSSON, Blacksburg, VA (US); Connor W. EDSALL, Blacksburg, VA (US)

(21) Appl. No.: 17/642,298

(22) PCT Filed: Sep. 19, 2020
(86) PCT No.: PCT/US2020/051681

§ 371 Date: Mar. 11, 2022

Related U.S. Application Data
(60) Provisional application No. 62/903,308, filed on Sep. 20, 2019.

(51) Int. Cl. A61B 5/055; A61B 90/00

(52) U.S. Cl. CPC A61B 5/055; A61B 90/37; A61B 2090/374

(57) ABSTRACT

Disclosed herein are devices, systems, and methods for use in a magnetic resonance imaging (MRI)-guided procedure in which focused energy is applied to an area of interest of a subject. Disclosed herein are coupling baths comprising an aqueous solution comprising a plurality of paramagnetic particles dispersed in water, wherein, when magnetic resonance images are collected from the area of interest of the subject for MRI guidance: the coupling bath is located proximate to the area of interest, and the composition, the average particle size, the shape, the concentration, the presence or absence of the capping layer, the identity of the plurality of ligands when the capping layer is present, the average thickness of the capping layer when the capping layer is present, or a combination thereof is/are selected such that the coupling bath reduces or prevents imaging artifacts in the magnetic resonance images for the MRI guidance.

### CROSS-REFERENCE TO RELATED APPLICATIONS

This application claims the benefit of priority to U.S. Provisional Application No. 62/903,308 filed Sep. 20, 2019, which is hereby incorporated herein by reference in its entirety.

### STATEMENT OF GOVERNMENT SUPPORT

This invention was made with government support under Grant No. TR003015 awarded by the National Institutes of Health. The government has certain rights in the invention.

### BACKGROUND

Coupling baths, nominally composed of degassed water, play important roles during magnetic resonance imaging (MRI)-guided procedures in which focused energy is applied to an area of interest of a subject, such as in transcranial focused ultrasound surgery. However, this large water bolus also degrades the quality of intraoperative magnetic resonance (MR) guidance imaging. A need exists for devices, systems, and methods for suppressing these image degradations while preserving compatibility with the application of the focused energy. The devices, systems, and methods discussed herein address these and other needs.

### SUMMARY

In accordance with the purposes of the disclosed devices, systems, and methods as embodied and broadly described herein, the disclosed subject matter relates to devices, systems, and methods for magnetic resonance imaging (MRI)-guided procedures.

For example, disclosed herein are coupling baths for use in a magnetic resonance imaging (MRI)-guided procedure in which focused energy is applied to an area of interest of a subject, the coupling bath comprising: an aqueous solution comprising a plurality of paramagnetic particles dispersed in water; each of the plurality of paramagnetic particles having a composition, an average particle size, a shape, a concentration, and an optional capping layer; the optional capping layer, when present, comprising a plurality of ligands attached to a surface of the paramagnetic particle and having an average thickness; wherein, when magnetic resonance images are collected from the area of interest of the subject for the MRI guidance: the coupling bath is located proximate to the area of interest, and the composition, the average particle size, the shape, the concentration, the presence or absence of the capping layer, the identity of the plurality of ligands when the capping layer is present, the average thickness of the capping layer when the capping layer is present, or a combination thereof is/are selected such that the coupling bath reduces or prevents imaging artifacts in the magnetic resonance images for the MRI guidance.

In some examples, the plurality of paramagnetic particles are biocompatible. In some examples, the plurality of paramagnetic particles comprise a metal selected from the group consisting of Fe, Mn, Ni, Gd, and combinations thereof. In some examples, the plurality of paramagnetic particles comprise Fe. In some examples, the plurality of paramagnetic particles comprise an iron oxide. In some examples, the plurality of paramagnetic particles comprise Fe₃O₄.

In some examples, the plurality of paramagnetic particles have an average particle size of 250 nanometers (nm) or less, 100 nm or less, or 50 nm or less. In some examples, the plurality of paramagnetic particles have an average particle size of from 30 nm to 50 nm. In some examples, the plurality of paramagnetic particles are substantially spherical in shape.

In some examples, each of the plurality of paramagnetic particles further comprises a capping layer comprising a plurality of ligands, the plurality of ligands being attached to a surface of each of the plurality of paramagnetic particles. In some examples, the plurality of ligands are hydrophilic such that the capping layer is hydrophilic. In some examples, the plurality of ligands comprise poly((meth)acrylic acid). In some examples, the capping layer has an average thickness of from 1 nm to 10 nm.

In some examples, the plurality of paramagnetic particles have a concentration of 10 mM or less, 5 mM or less, or 1 mM or less in the coupling bath.

In some examples, the coupling bath has an r2 relaxivity of 20 per second per mM or more and/or an R2 relaxation rate of 20 per second or more when subjected to multi-spin-echo measurements on a 1.5 T or 3 T MRI scanner. In some examples, the coupling bath has a T2 relaxation time of 50 ms or less when subjected to multi-spin-echo measurements on a 1.5 T or 3 T MRI scanner. In some examples, the coupling bath has a probability of cavitation is 0.5 or less for a peak negative pressure of 30 mega pascals (MPa) or less when subjected to highly focused, shocked, 5 cycle pulses at a pulse rate of 1 Hz. In some examples, the coupling bath has a cavitation duty cycle of 0.5 or less for a peak negative pressure of 30 MPa or less when subjected to continuous wave acoustic sonications with acoustic powers of from 1 to 1000 W for 10 seconds. In some examples, the coupling bath has an MR signal in a T2 and/or T2*-weighted image that is less than the corresponding MR signal from a corresponding coupling bath in the absence of the plurality of paramagnetic particles by 50% or more, 75% or more, 90% or more, 95% or more, or 99% or more. In some examples, the coupling bath has a peak temperature change that is different than the corresponding peak temperature change for a corresponding coupling bath in the absence of the plurality of paramagnetic particles by 10% or less, 5% or less, or 1% or less, when insonated for 10 seconds at an acoustic power of from 50 to 1000 W. In some examples, the coupling bath has a peak temperature change that is different than the corresponding peak temperature change for a corresponding coupling bath in the absence of the plurality of paramagnetic particles by 5° C. or less, when insonated for 10 seconds at an acoustic power of from 50 to 1000 W. In some examples, the coupling bath has similar mechanical, thermal, acoustic, and/or electromagnetic properties as a corresponding coupling bath in the absence of the plurality of paramagnetic particles.

Also disclosed herein are methods for a magnetic resonance imaging (MRI)-guided procedure on a subject wherein focused energy is applied to an area of interest of the subject, the method comprising providing the any of the coupling baths described herein proximate the area of interest, such that, when magnetic resonance images are collected from the area of interest of the subject for the MRI guidance, the coupling bath reduces or prevents imaging artifacts in the magnetic resonance images for the MRI guidance. In some examples, the use of the coupling bath improves the image-guidance and/or safety of the MRI guided procedure relative to a corresponding coupling bath in the absence of the plurality of paramagnetic particles. In some examples, the method further comprises collecting magnetic resonance images from the area of interest of the subject for the MRI guidance. In some examples, the magnetic resonance images for the MRI guidance are collected intraoperatively. In some examples, the methods further comprise circulating the coupling bath during the collection of the magnetic resonance images for the MRI guidance.

In some examples, the MRI-guided procedure comprises MRI-guided microwave ablation, MRI-guided laser interstitial surgery, MRI-guided focused ultrasound (FUS), or a combination thereof.

In some examples, the focused energy applied to the area of interest in the MRI-guided procedure comprises focused ultrasound (FUS). In some examples, the MRI-guided procedure is a transcranial focused ultrasound (T-FUS) procedure. In some examples, the coupling bath is acoustically compatible with clinical transcranial FUS procedures. In some examples, the prefocal acoustic field is below the 0.5 cavitation probability threshold of the coupling bath. In some examples, the prefocal acoustic field is below the non-trivial cavitation probability threshold of the coupling bath.

Also disclosed herein are systems for a magnetic resonance imaging (MRI)-guided procedure on a subject wherein focused energy is applied to an area of interest of the subject, the system comprising any of the coupling baths described herein proximate the area of interest and a magnetic resonance imaging device configured to collect images from the area of interest of the subject for the MRI guidance, wherein when the magnetic resonance images are collected from the area of interest of the subject for MRI guidance, the coupling bath reduces or prevents imaging artifacts in the magnetic resonance images for the MRI guidance.

In some examples, the system further comprises a device configured to apply focused energy to the area of interest in the subject. In some examples, the device configured to apply focused energy to the area of interest in the subject comprises a transcranial focused ultrasound (T-FUS) device. In some examples, the T-FUS device comprises a transducer and the coupling bath is located between the transducer and the area of interest of the subject.

In some examples, the system further comprises a means for circulating the coupling bath.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The details of one or more embodiments of the invention are set forth in the accompanying drawings and description below. Other features, object, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 is a system diagram illustrating an imaging system capable of implementing aspects of the present disclosure in accordance with one or more embodiments.

FIG. 2 is a diagram showing one example embodiment of a system with thermal therapy used with MRI, which is capable of implementing aspects of the present disclosure in accordance with one or more embodiments.

FIG. 3 is a diagram showing another example embodiment of a system with thermal therapy used with MRI, which is capable of implementing aspects of the present disclosure in accordance with one or more embodiments.

FIG. 4 is a basic, schematic representation of an ultrasound system.

FIG. 5 illustrates an example computing device.

FIG. 6A is a schematic setup of a transcranial focused ultrasound (T-FUS) procedure. Under MR guidance, acoustic pulses (red lines) propagate from the transducer surface, through the coupling bath (blue), and into the patient.

FIG. 6B-FIG. 6D show example imaging artifacts. FIG. 6B shows that if the imaging field of view is too small, the large bolus of the coupling bath can alias into the anatomy of interest (red arrow). FIG. 6C shows that the large MR signal of the coupling bath can skew pre-scan calibrations, causing sub-optimal RF calibration (red arrow). FIG. 6D shows that vibration and flow effects can cause MR signal from the coupling bath to superimpose onto the anatomy of interest (red arrow).

FIG. 7 is a schematic of Experiments 1 and 2. The bowl of a clinical, hemispherical transducer array was filled with various aqueous, suspended magnetite nanoparticle coupling baths and imaged by MRI. The transducer also insonated a gel target while MRI thermometry monitored thermal deposition.

FIG. 8 is a schematic of Experiment 3. The transducer in FIG. 7 was filled with degassed water and used to insonate various aqueous, suspended magnetite nanoparticle mixtures suspended in a small holder and backed by an acoustic absorber. PCD detectors were used to record cavitation activity.

FIG. 9 is a schematic of Experiment 4. A shock-scattering histotripsy transducer (Tx) was placed in a water tank and directly insonated various coupling baths located in an acoustically transparent holder. A high speed camera and passive cavitation detector (PCD) were used to record cavitation activity.

FIG. 10 is an example anatomical (spin-echo) image acquired while ablating a gel target using a 650 kHz T-FUS system and using a 0 mM nanoparticle concentration in the coupling bath.

FIG. 11 is an example anatomical (spin-echo) image acquired while ablating a gel target using a 650 kHz T-FUS system and using a 0.25 mM nanoparticle concentration in the coupling bath. The 0.25 mM nanoparticle concentration suppresses the water bath signal by 98% in the spin-echo image.

FIG. 12 is an example thermometry (gradient-echo) image acquired while ablating a gel target using a 650 kHz T-FUS system and using a 0 mM nanoparticle concentration in the coupling bath.

FIG. 13 is an example thermometry (gradient-echo) image acquired while ablating a gel target using a 650 kHz T-FUS system and using a 0.25 mM nanoparticle concentration in the coupling bath. The 0.25 mM nanoparticle concentration suppresses the water bath signal by 90% in the gradient-echo image.

FIG. 14 is the signal magnitude in the coupling bath in the turbo spin echo images relative to that of the gel as a function of nanoparticle concentration. The coupling bath signal magnitude decreases with an apparently multi-exponential curve, with the majority of signal loss occurring in the first concentration step.

FIG. 15-FIG. 17 are example HASTE images of a homogeneous gel target in a hemispherical transducer filled with continuously circulating coupling baths containing varying concentrations of magnetite nanoparticles. Water motion causes MR signal from the coupling bath to incoherently superimpose onto the gel.

FIG. 18 is a plot of the ratio of the standard deviation of the ROI's (green ellipses). The gel is ordinarily quite homogeneous and motion artifact creates spatial fluctuations that increase the relative standard deviation in an ROI. The nanoparticles effectively suppress these fluctuations with the largest improvement caused by the first concentration increment.

FIG. 19 is the mean and standard deviation of the temperatures in a gel target placed at the focus of a 650 kHz T-FUS transducer as a function of transmitted acoustic power while using 10 s sonications for two coupling bath compositions. The 0.25 mM nanoparticle concentration coupling bath caused a roughly 5% (˜2° C.) decrease in peak temperature across all acoustic powers above 200 W. Data points are slightly shifted horizontally to increase figure legibility.

FIG. 20 is example cavitation duty cycle curves, fit to the observed relative frequency of spectra containing cavitation emissions, observed while insonating coupling baths containing various concentrations of magnetite nanoparticles in a high duty cycle regime.

FIG. 21 is the 0.5 cavitation duty cycle threshold power, derived from all fits, including those shown in FIG. 20, as a function of nanoparticle concentration. This threshold decreases linearly with pressure at a rate of 308 WmM⁻¹.

FIG. 22 is the cavitation dose estimated for each acoustic power level for several coupling bath compositions. As nanoparticle concentration increases, the number and spectral power of cavitation events increases, increasing thermal dose.

FIG. 23 is example cavitation probability curves, fit to the relative frequency of cavitation events, as a function of peak negative pressure of acoustic pulses, observed while insonating coupling baths containing various concentrations of magnetite nanoparticles in a low duty cycle regime.

FIG. 24 is the 0.5 cavitation probability threshold pressure, derived from all fits computed from this insonation scheme, including those shown in FIG. 23, as a function of nanoparticle concentration. The nanoparticles reduce this threshold by 15±7%. For comparison, the high duty cycle power thresholds presented FIG. 20-FIG. 22 are converted into pressure and reproduced here.

FIG. 25 is a schematic setup of a T-FUS procedure, showing acoustic propagation (red) through a conducting water bath (blue).

FIG. 26 shows imaging errors caused by too small an imaging field-of-view. Water signal superimposes onto and obscures the desired patient signal (arrow).

FIG. 27 shows water vibration and flow causes imaging errors in the water bath and the patient (arrows).

FIG. 28A is an anatomical image of a gel target using a clinical T-FUS system with the water bath under circulation (worst case scenario) using a 0 mM magnetite suspension in the coupling bath.

FIG. 28B is an anatomical image of a gel target using a clinical T-FUS system with the water bath under circulation (worst case scenario) using a 0.5 mM magnetite suspension in the coupling bath. The 0.5 mM suspension effectively removes motion-induced imaging errors (arrow).

FIG. 28C is a thermometry image of a gel target taken with a 50% reduced imaging field-of-view (15 cm) with a 0 mM magnetite suspension in the coupling bath.

FIG. 28D is a thermometry image of a gel target taken with a 50% reduced imaging field-of-view (15 cm) with a 0.5 mM magnetite suspension in the coupling bath. The magnetite suspension eliminates superimposing water bath signals.

FIG. 29 shows the cavitation threshold pressures for aqueous magnetite suspensions as a function of magnetite concentration. The particles decrease the magnitude of this threshold relative to water.

FIG. 30 shows the cavitation score (relative average spectral power over the first subharmonic) detected in water baths with varying concentrations of magnetite nanoparticles when exposed to 650 kHz, 10 s sonications at the natural focus of a hemispherical T-FUS transducer. The particles enhance subharmonic emissions over a broad range of transmitted acoustic powers.

FIG. 31 shows example cavitation probability curves as a function of peak negative pressures (PNP) deposited in 0, 0.12, and 2.5 mM concentration of aqueous magnetite particles when subjected to 5 cycle, acoustic pulses fired with a 1 Hz pulse repetition rate. The particles demonstrably lower the cavitation threshold relative to water.

FIG. 32 shows T2-weighted images of a human volunteer coupled to a 650 kHz transcranial T-FUS system as a function of the concentration of magnetite nanoparticles in the water bath. The nanoparticles effectively suppress the water signal and remove the aliasing artifact located near the patient's anterior and posterior.

FIG. 33 shows the T2 relaxation curves for samples with varying particle concentrations.

FIG. 34 shows the T1 relaxation curves for samples with varying particle concentrations.

FIG. 35 shows the r2 relaxivity as a function of particle concentration calculated from the results in FIG. 33.

FIG. 36 shows the r1 relaxivity as a function of particle concentration calculated from the results in FIG. 34.

FIG. 37 shows the results of pulsed cavitation threshold tests.

FIG. 38 results of continuous wave cavitation threshold experiments for “T” dilution.

FIG. 39 results of continuous wave cavitation threshold experiments for “U” dilution.

FIG. 40 plot of the 0.5 cavitation duty cycle threshold pressures as both a function iron concentration and measured dissolved oxygen content for the “T” dilution.

FIG. 41 plot of the 0.5 cavitation duty cycle threshold pressures as both a function iron concentration and measured dissolved oxygen content for the “U” dilution.

### DETAILED DESCRIPTION

The devices, systems, and methods described herein may be understood more readily by reference to the following detailed description of specific aspects of the disclosed subject matter and the Examples included therein.

Before the present devices, systems, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include any applicable human, animal, or other organism, living or dead, or other biological or molecular structure or chemical environment, and may relate to particular components of the subject, for instance specific organs, bones, tissues, or fluids, which may be in a particular location of the subject referred to herein as an “area of interest” or a “region of interest.” For example, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “(meth)acryl . . . ” includes “acryl . . . ,” “methacryl . . . ,” or mixtures thereof.

Disclosed herein are devices, systems, and methods for use in a magnetic resonance imaging (MRI)-guided procedure in which focused energy is applied to an area of interest of a subject.

For example, disclosed herein are coupling baths for use in a magnetic resonance imaging (MRI)-guided procedure in which focused energy is applied to an area of interest of a subject. The coupling bath can comprise an aqueous solution comprising a plurality of paramagnetic particles dispersed in water (e.g., degassed water). The coupling baths disclosed herein for use in a magnetic resonance imaging (MRI)-guided procedure in which focused energy is applied to an area of interest of a subject, the coupling baths comprising an aqueous solution comprising a plurality of paramagnetic particles dispersed in water; each of the plurality of paramagnetic particles having a composition, an average particle size, a shape, a concentration, and an optional capping layer; the optional capping layer, when present, comprising a plurality of ligands attached to a surface of the paramagnetic particle and having an average thickness; wherein, when magnetic resonance images are collected from the area of interest of the subject for the MRI guidance: the coupling bath is located proximate to the area of interest, and the composition, the average particle size, the shape, the concentration, the presence or absence of the capping layer, the identity of the plurality of ligands when the capping layer is present, the average thickness of the capping layer when the capping layer is present, or a combination thereof is/are selected such that the coupling bath reduces or prevents imaging artifacts in the magnetic resonance images for the MRI guidance. For example, the coupling baths disclosed herein can reduce or prevent imaging artifacts in the magnetic resonance images for the MRI guidance relative to magnetic resonance images collected using a corresponding coupling bath in the absence of the plurality of paramagnetic particles, e.g. a coupling bath consisting essentially of or consisting of water (e.g., degassed water).

The plurality of paramagnetic particles can, in some examples, be biocompatible. In some examples, the plurality of paramagnetic particles comprise a metal selected from the group consisting of Fe, Mn, Ni, Gd, Cu, Co, V, and combinations thereof. In some examples, the plurality of paramagnetic particles comprise a metal selected from the group consisting of Fe, Mn, Ni, Gd, and combinations thereof. In some examples, the plurality of paramagnetic particles comprise Fe. The plurality of paramagnetic particles can, for example, comprise an iron oxide, such as Fe₃O₄.

The plurality of paramagnetic particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The plurality of paramagnetic particles have, for example, an average particle size of 250 nanometers (nm) or less (e.g., 225 nm or less, 200 nm or less, 175 nm or less, 150 nm or less, 125 nm or less, 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, 15 nm or less, 10 nm or less, or 5 nm or less). In some examples, the plurality of paramagnetic particles can have an average particle size of 1 nm or more (e.g., 2 nm or more, 3 nm or more, 4 nm or more, 5 nm or more, 10 nm or more, 15 nm or more, 20 nm or more, 25 nm or more, 30 nm or more, 35 nm or more, 40 nm or more, 45 nm or more, 50 nm or more, 55 nm or more, 60 nm or more, 65 nm or more, 70 nm or more, 75 nm or more, 80 nm or more, 85 nm or more, 90 nm or more, 95 nm or more, 100 nm or more, 125 nm or more, 150 nm or more, 175 nm or more, or 200 nm or more). The average particle size of the plurality of paramagnetic particles can range from any of the minimum values described above to any of the maximum values described above. For example, the plurality of paramagnetic particles can have an average particle size of from 1 nm to 250 nm (e.g., from 1 nm to 125 nm, from 125 nm to 250 nm, from 1 nm to 50 nm, from 50 nm to 100 nm, from 100 nm to 150 nm, from 150 nm to 200 nm, from 200 nm to 250 nm, from 1 nm to 200 nm, from 1 nm to 150 nm, from 1 nm to 100 nm, from 10 nm to 250 nm, from 10 nm to 100 nm, from 10 nm to 50 nm, from 20 nm to 50 nm, or from 30 nm to 50 nm).

In some examples, the plurality of paramagnetic particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

The plurality of paramagnetic particles can comprise particles of any shape (e.g., a sphere, a rod, a quadrilateral, an ellipse, a triangle, a polygon, etc.). In some examples, the plurality of paramagnetic particles can have a regular shape, an irregular shape, an isotropic shape, an anisotropic shape, or a combination thereof. In some examples, the plurality of paramagnetic particles are substantially spherical.

In some examples, each of the plurality of paramagnetic particles further comprises a capping layer comprising a plurality of ligands, the plurality of ligands being attached to a surface of each of the plurality of paramagnetic particles. Suitable ligands for capping layers for particles are known in the art. The capping layer can be used to stabilize the plurality of paramagnetic particles. The capping layer can, for example, prevent oxidation on the surface of the plurality of paramagnetic particles and/or to increase the dispersibility of the plurality of paramagnetic particles.

In some examples, the ligands can be attached to the surface of the paramagnetic particles, for example, by coordination bonds. Ligands can also be associated with the paramagnetic particles via non-covalent interactions. In some examples, the ligands can individually be selected to be hydrophilic, hydrophobic, or amphiphilic. In addition, the plurality of ligands can, in combination, be selected so as to provide a shell surrounding each of the plurality of paramagnetic particles, the shell being hydrophilic, hydrophobic, or amphiphilic. In some examples, the plurality of ligands are hydrophilic such that the capping layer is hydrophilic. The ligands can comprise coordinating or bonding functional groups, such as thiol, amine, phosphine, phosphate, amide, ether, alkene, chloride, hydride, carboxyl, alkyl, sulfate, and derivatives thereof, and combinations thereof. In some examples, the ligands can comprise chelating agents and/or polydentate ligands. Examples of ligands include, but are not limited to, citrate, tannic acid, lipoic acid, dodecane thiol, cetyl trimethyl ammonium bromide (CTAB), cetyl trimethyl ammonium chloride (CTAC), branched polyethylenimine (BPEI), polyethylene glycol (PEG), polyvinylpyrrolidone (PVP), sodium dodecyl sulfate (SDS), poly((meth)acrylic acid), and combinations thereof. In some examples, the plurality of ligands comprise poly((meth)acrylic acid).

The capping layer can, for example, have an average thickness of 1 nm or more (e.g., 1.5 nm or more, 2 nm or more, 2.5 nm or more, 3 nm or more, 3.5 nm or more, 4 nm or more, 4.5 nm or more, 5 nm or more, 5.5 nm or more, 6 nm or more, 6.5 nm or more, 7 nm or more, 7.5 nm or more, 8 nm or more, 8.5 nm or more, or 9 nm or more). In some examples, the capping layer can have an average thickness of 10 nm or less (e.g., 9.5 nm or less, 9 nm or less, 8.5 nm or less, 8 nm or less, 7.5 nm or less, 7 nm or less, 6.5 nm or less, 6 nm or less, 5.5 nm or less, 5 nm or less, 4.5 nm or less, 4 nm or less, 3.5 nm or less, 3 nm or less, 2.5 nm or less, 2 nm or less, or 1.5 nm or less). The average thickness of the capping layer can range from any of the minimum values described above to any of the maximum values described above. For example, the capping layer can have an average thickness of from 1 nm to 10 nm (e.g., from 1 nm to 5 nm, from 5 nm to 10 nm, from 1 nm to 3 nm, from 3 nm to 6 nm, from 6 nm to 9 nm, from 1 nm to 9 nm, from 2 nm to 10 nm, from 2 nm to 9 nm, from 1 nm to 8 nm, from 1 nm to 7 nm, or from 1 nm to 6 nm). The average thickness of the capping layer can, for example, be measured via transmission electron microscopy and/or dynamic light scattering (DLS).

The plurality of paramagnetic particles can, for example, have a concentration of 10 millimolar (mM) or less in the coupling bath (e.g., 9.5 mM or less, 9 mM or less, 8.5 mM or less, 8 mM or less, 7.5 mM or less, 7 mM or less, 6.5 mM or less, 6 mM or less, 5.5 mM or less, 5 mM or less, 4.5 mM or less, 4 mM or less, 3.5 mM or less, 3 mM or less, 2.5 mM or less, 2 mM or less, 1.5 mM or less, 1 mM or less, 0.95 mM or less, 0.9 mM or less, 0.85 mM or less, 0.8 mM or less, 0.75 mM or less, 0.7 mM or less, 0.65 mM or less, 0.6 mM or less, 0.55 mM or less, 0.5 mM or less, 0.45 mM or less, 0.4 mM or less, 0.35 mM or less, 0.3 mM or less, 0.25 mM or less, 0.2 mM or less, 0.15 mM or less, 0.1 mM or less, 0.09 mM or less, 0.08 mM or less, 0.07 mM or less, 0.06 mM or less, 0.05 mM or less, 0.04 mM or less, 0.03 mM or less, 0.02 mM or less, 0.01 mM or less, 0.009 mM or less, 0.008 mM or less, 0.007 mM or less, 0.006 mM or less, 0.005 mM or less, 0.004 mM or less, 0.003 mM or less, or 0.002 mM or less). In some examples, the plurality of paramagnetic particles can have a concentration of 1 micromolar (μM) or more in the coupling bath (e.g., 0.002 mM or more, 0.003 mM or more, 0.004 mM or more, 0.005 mM or more, 0.006 mM or more, 0.007 mM or more, 0.008 mM or more, 0.009 mM or more, 0.01 mM or more, 0.02 mM or more, 0.03 mM or more, 0.04 mM or more, 0.05 mM or more, 0.06 mM or more, 0.07 mM or more, 0.08 mM or more, 0.09 mM or more, 0.1 mM or more, 0.15 mM or more, 0.2 mM or more, 0.25 mM or more, 0.3 mM or more, 0.35 mM or more, 0.4 mM or more, 0.45 mM or more, 0.5 mM or more, 0.55 mM or more, 0.6 mM or more, 0.65 mM or more, 0.7 mM or more, 0.75 mM or more, 0.8 mM or more, 0.85 mM or more, 0.9 mM or more, 0.95 mM or more, 1 mM or more, 1.5 mM or more, 2 mM or more, 2.5 mM or more, 3 mM or more, 3.5 mM or more, 4 mM or more, 4.5 mM or more, 5 mM or more, 5.5 mM or more, 6 mM or more, 6.5 mM or more, 7 mM or more, 7.5 mM or more, 8 mM or more, 8.5 mM or more, or 9 mM or more). The concentration of the plurality of paramagnetic particles in the coupling bath can range from any of the minimum values described above to any of the maximum values described above. For example, the plurality of paramagnetic particles can have a concentration of from 1 μM to 10 mM in the coupling bath (e.g., from 0.001 mM to 0.01 mM, from 0.01 mM to 0.1 mM, from 0.1 mM to 1 mM, from 0.001 mM to 0.1 mM, from 0.01 mM to 1 mM, from 1 mM to 10 mM, or from 0.01 mM to 1 mM).

The plurality of paramagnetic particles can, in some examples, comprise a metal and the concentration of the plurality of paramagnetic particles in the coupling bath can be selected such that the concentration of the metal comprising the plurality of paramagnetic particles is 10 millimolar (mM) or less in the coupling bath (e.g., 9.5 mM or less, 9 mM or less, 8.5 mM or less, 8 mM or less, 7.5 mM or less, 7 mM or less, 6.5 mM or less, 6 mM or less, 5.5 mM or less, 5 mM or less, 4.5 mM or less, 4 mM or less, 3.5 mM or less, 3 mM or less, 2.5 mM or less, 2 mM or less, 1.5 mM or less, 1 mM or less, 0.95 mM or less, 0.9 mM or less, 0.85 mM or less, 0.8 mM or less, 0.75 mM or less, 0.7 mM or less, 0.65 mM or less, 0.6 mM or less, 0.55 mM or less, 0.5 mM or less, 0.45 mM or less, 0.4 mM or less, 0.35 mM or less, 0.3 mM or less, 0.25 mM or less, 0.2 mM or less, 0.15 mM or less, 0.1 mM or less, 0.09 mM or less, 0.08 mM or less, 0.07 mM or less, 0.06 mM or less, 0.05 mM or less, 0.04 mM or less, 0.03 mM or less, 0.02 mM or less, 0.01 mM or less, 0.009 mM or less, 0.008 mM or less, 0.007 mM or less, 0.006 mM or less, 0.005 mM or less, 0.004 mM or less, 0.003 mM or less, or 0.002 mM or less). In certain examples, the plurality of paramagnetic particles comprise a metal and the concentration of the plurality of paramagnetic particles in the coupling bath can be selected such that the concentration of the metal comprising the plurality of paramagnetic particles is 1 micromolar (μM) or more in the coupling bath (e.g., 0.002 mM or more, 0.003 mM or more, 0.004 mM or more, 0.005 mM or more, 0.006 mM or more, 0.007 mM or more, 0.008 mM or more, 0.009 mM or more, 0.01 mM or more, 0.02 mM or more, 0.03 mM or more, 0.04 mM or more, 0.05 mM or more, 0.06 mM or more, 0.07 mM or more, 0.08 mM or more, 0.09 mM or more, 0.1 mM or more, 0.15 mM or more, 0.2 mM or more, 0.25 mM or more, 0.3 mM or more, 0.35 mM or more, 0.4 mM or more, 0.45 mM or more, 0.5 mM or more, 0.55 mM or more, 0.6 mM or more, 0.65 mM or more, 0.7 mM or more, 0.75 mM or more, 0.8 mM or more, 0.85 mM or more, 0.9 mM or more, 0.95 mM or more, 1 mM or more, 1.5 mM or more, 2 mM or more, 2.5 mM or more, 3 mM or more, 3.5 mM or more, 4 mM or more, 4.5 mM or more, 5 mM or more, 5.5 mM or more, 6 mM or more, 6.5 mM or more, 7 mM or more, 7.5 mM or more, 8 mM or more, 8.5 mM or more, or 9 mM or more). The concentration of the plurality of paramagnetic particles in the coupling bath can be selected such that the concentration of the metal comprising the plurality of paramagnetic particles can range from any of the minimum values described above to any of the maximum values described above. For example, the plurality of paramagnetic particles can comprise a metal and the concentration of the plurality of paramagnetic particles in the coupling bath can be selected such that the concentration of the metal comprising the plurality of paramagnetic particles is from 1 μM to 10 mM in the coupling bath (e.g., from 0.001 mM to 0.01 mM, from 0.01 mM to 0.1 mM, from 0.1 mM to 1 mM, from 0.001 mM to 0.1 mM, from 0.01 mM to 1 mM, from 1 mM to 10 mM, or from 0.01 mM to 1 mM).

The size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected in view of a variety of factors. For example, the size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected such that the coupling bath reduces or prevents imaging artifacts in the magnetic resonance images for the MRI guidance. In some examples, the size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected such that the magnetic resonance images for the MRI guidance are improved relative to those collected in the presence of a corresponding coupling bath in the absence of the plurality of paramagnetic particles, e.g., in a coupling bath consisting essentially of or consisting of water (e.g., degassed water). In some examples, the size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected such that the coupling bath is has little to no MR signal (e.g., such that the coupling bath is effectively invisible to MRI scans). In some examples, the size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected such that the coupling bath improves the image-guidance and/or safety of the MRI guided procedure relative to a corresponding coupling bath in the absence of the plurality of paramagnetic particles, e.g. a coupling bath consisting essentially of or consisting of water (e.g., degassed water).

The coupling bath can, for example, have an r2 relaxivity of 20 per second per mM or more (e.g., 25 s⁻¹ mM⁻¹ or more; 30 s⁻¹ mM⁻¹ or more; 35 s⁻¹ mM⁻¹ or more; 40 s⁻¹ mM⁻¹ or more; 45 s⁻¹ mM⁻¹ or more; 50 s⁻¹ mM⁻¹ or more; 60 s⁻¹ mM⁻¹ or more; 70 s⁻¹ mM⁻¹ or more; 80 s⁻¹ mM⁻¹ or more; 90 s⁻¹ mM⁻¹ or more; 100 s⁻¹ mM⁻¹ or more; 125 s⁻¹ mM⁻¹ or more; 150 s⁻¹ mM⁻¹ or more; 175 s⁻¹ mM⁻¹ or more; 200 s⁻¹ mM⁻¹ or more; 225 s⁻¹ mM⁻¹ or more; 250 s⁻¹ mM⁻¹ or more; 275 s⁻¹ mM⁻¹ or more; 300 s⁻¹ mM⁻¹ or more; 325 s⁻¹ mM⁻¹ or more; 350 s⁻¹ mM⁻¹ or more; 375 s⁻¹ mM⁻¹ or more; 400 s⁻¹ mM⁻¹ or more; 425 s⁻¹ mM⁻¹ or more; 450 s⁻¹ mM⁻¹ or more; 475 s⁻¹ mM⁻¹ or more; 500 s⁻¹ mM⁻¹ or more; 550 s⁻¹ mM⁻¹ or more; 600 s⁻¹ mM⁻¹ or more; 650 s⁻¹ mM⁻¹ or more; 700 s⁻¹ mM⁻¹ or more; 750 s⁻¹ mM⁻¹ or more; 800 s⁻¹ mM⁻¹ or more; 850 s⁻¹ mM⁻¹ or more; 900 s⁻¹ mM⁻¹ or more; 950 s⁻¹ mM⁻¹ or more; 1,000 s⁻¹ mM⁻¹ or more; 1,100 s⁻¹ mM⁻¹ or more; 1,200 s⁻¹ mM⁻¹ or more; 1,300 s⁻¹ mM⁻¹ or more; 1,400 s⁻¹ mM⁻¹ or more; 1,500 s⁻¹ mM⁻¹ or more; 1,600 s⁻¹ mM⁻¹ or more; 1,700 s⁻¹ mM⁻¹ or more; 1,800 s⁻¹ mM⁻¹ or more; 1,900 s⁻¹ mM⁻¹ or more; 2,000 s⁻¹ mM⁻¹ or more; 2,250 s⁻¹ mM⁻¹ or more; 2,500 s⁻¹ mM⁻¹ or more; 2,750 s⁻¹ mM⁻¹ or more; 3,000 s⁻¹ mM⁻¹ or more; 3,250 s⁻¹ mM⁻¹ or more; 3,500 s⁻¹ mM⁻¹ or more; 3,750 s⁻¹ mM⁻¹ or more; 4,000 s⁻¹ mM⁻¹ or more; 4,250 s⁻¹ mM⁻¹ or more; 4,500 s⁻¹ mM⁻¹ or more; 5,000 s⁻¹ mM⁻¹ or more; 5,500 s⁻¹ mM⁻¹ or more; 6,000 s⁻¹ mM⁻¹ or more; 6,500 s⁻¹ mM⁻¹ or more; 7,000 s⁻¹ mM⁻¹ or more; 7,500 s⁻¹ mM⁻¹ or more; 8,000 s⁻¹ mM⁻¹ or more; 8,500 s⁻¹ mM⁻¹ or more; 9,000 s⁻¹ mM⁻¹ or more; 9,500 s⁻¹ mM⁻¹ or more; 10,000 s⁻¹ mM⁻¹ or more; 11,000 s⁻¹ mM⁻¹ or more; 12,000 s⁻¹ mM⁻¹ or more; 13,000 s⁻¹ mM⁻¹ or more; 14,000 s⁻¹ mM⁻¹ or more; 15,000 s⁻¹ mM⁻¹ or more; 16,000 s⁻¹ mM⁻¹ or more; 17,000 s⁻¹ mM⁻¹ or more; 18,000 s⁻¹ mM⁻¹ or more; or 19,000 s⁻¹ mM⁻¹ or more) when subjected to multi-spin-echo measurements on a 1.5 T or 3 T MRI scanner.

In some examples, the coupling bath can have an r2 relaxivity of 20,000 s⁻¹ mM⁻¹ or less (e.g., 19,000 s⁻¹ mM⁻¹ or less; 18,000 s⁻¹ mM⁻¹ or less; 17,000 s⁻¹ mM⁻¹ or less; 16,000 s⁻¹ mM⁻¹ or less; 15,000 s⁻¹ mM⁻¹ or less; 14,000 s⁻¹ mM⁻¹ or less; 13,000 s⁻¹ mM⁻¹ or less; 12,000 s⁻¹ mM⁻¹ or less; 11,000 s⁻¹ mM⁻¹ or less; 10,000 s⁻¹ mM⁻¹ or less; 9,500 s⁻¹ mM⁻¹ or less; 9,000 s⁻¹ mM⁻¹ or less; 8,500 s⁻¹ mM⁻¹ or less; 8,000 s⁻¹ mM⁻¹ or less; 7,500 s⁻¹ mM⁻¹ or less; 7,000 s⁻¹ mM⁻¹ or less; 6,500 s⁻¹ mM⁻¹ or less; 6,000 s⁻¹ mM⁻¹ or less; 5,500 s⁻¹ mM⁻¹ or less; 5,000 s⁻¹ mM⁻¹ or less; 4,500 s⁻¹ mM⁻¹ or less; 4,250 s⁻¹ mM⁻¹ or less; 4,000 s⁻¹ mM⁻¹ or less; 3,750 s⁻¹ mM⁻¹ or less; 3,500 s⁻¹ mM⁻¹ or less; 3,250 s⁻¹ mM⁻¹ or less; 3,000 s⁻¹ mM⁻¹ or less; 2,750 s⁻¹ mM⁻¹ or less; 2,500 s⁻¹ mM⁻¹ or less; 2,250 s⁻¹ mM⁻¹ or less; 2,000 s⁻¹ mM⁻¹ or less; 1,900 s⁻¹ mM⁻¹ or less; 1,800 s⁻¹ mM⁻¹ or less; 1,700 s⁻¹ mM⁻¹ or less; 1,600 s⁻¹ mM⁻¹ or less; 1,500 s⁻¹ mM⁻¹ or less; 1,400 s⁻¹ mM⁻¹ or less; 1,300 s⁻¹ mM⁻¹ or less; 1,200 s⁻¹ mM⁻¹ or less; 1,100 s⁻¹ mM⁻¹ or less; 1,000 s⁻¹ mM⁻¹ or less; 950 s⁻¹ mM⁻¹ or less; 900 s⁻¹ mM⁻¹ or less; 850 s⁻¹ mM⁻¹ or less; 800 s⁻¹ mM⁻¹ or less; 750 s⁻¹ mM⁻¹ or less; 700 s⁻¹ mM⁻¹ or less; 650 s⁻¹ mM⁻¹ or less; 600 s⁻¹ mM⁻¹ or less; 550 s⁻¹ mM⁻¹ or less; 500 s⁻¹ mM⁻¹ or less; 475 s⁻¹ mM⁻¹ or less; 450 s⁻¹ mM⁻¹ or less; 425 s⁻¹ mM⁻¹ or less; 400 s⁻¹ mM⁻¹ or less; 375 s⁻¹ mM⁻¹ or less; 350 s⁻¹ mM⁻¹ or less; 325 s⁻¹ mM⁻¹ or less; 300 s⁻¹ mM⁻¹ or less; 275 s⁻¹ mM⁻¹ or less; 250 s⁻¹ mM⁻¹ or less; 225 s⁻¹ mM⁻¹ or less; 200 s⁻¹ mM⁻¹ or less; 175 s⁻¹ mM⁻¹ or less; 150 s⁻¹ mM⁻¹ or less; 125 s⁻¹ mM⁻¹ or less; 100 s⁻¹ mM⁻¹ or less; 90 s⁻¹ mM⁻¹ or less; 80 s⁻¹ mM⁻¹ or less; 70 s⁻¹ mM⁻¹ or less; 60 s⁻¹ mM⁻¹ or less; 50 s⁻¹ mM⁻¹ or less; 45 s⁻¹ mM⁻¹ or less; 40 s⁻¹ mM⁻¹ or less; 35 s⁻¹ mM⁻¹ or less; or 30 s⁻¹ mM⁻¹ or less) when subjected to multi-spin-echo measurements on a 1.5 T or 3 T MRI scanner.

The r2 relaxivity of the coupling bath can range from any of the minimum values described above to any of the maximum values described above. For example, the coupling bath can have an r2 relaxivity of from 20 s⁻¹ mM⁻¹ to 20,000 s⁻¹ mM⁻¹ (e.g., from 20 s⁻¹ mM⁻¹ to 200 s⁻¹ mM⁻¹; from 200 s⁻¹ mM⁻¹ to 2,000 s⁻¹ mM⁻¹; from 2,000 s⁻¹ mM⁻¹ to 20,000 s⁻¹ mM⁻¹; from 50 s⁻¹ mM⁻¹ to 20,000 s⁻¹ mM⁻¹; or from 200 s⁻¹ mM⁻¹ to 20,000 s⁻¹ mM⁻¹) when subjected to multi-spin-echo measurements on a 1.5 T or 3 T MRI scanner.

In some examples, the coupling bath can have a R2 relaxation rate of 20 per second per mM or more (e.g., 25 s⁻¹ or more; 30 s⁻¹ or more; 35 s⁻¹ or more; 40 s⁻¹ or more; 45 s⁻¹ or more; 50 s⁻¹ or more; 60 s⁻¹ or more; 70 s⁻¹ or more; 80 s⁻¹ or more; 90 s⁻¹ or more; 100 s⁻¹ or more; 125 s⁻¹ or more; 150 s⁻¹ or more; 175 s⁻¹ or more; 200 s⁻¹ or more; 225 s⁻¹ or more; 250 s⁻¹ or more; 275 s⁻¹ or more; 300 s⁻¹ or more; 325 s⁻¹ or more; 350 s⁻¹ or more; 375 s⁻¹ or more; 400 s⁻¹ or more; 425 s⁻¹ or more; 450 s⁻¹ or more; 475 s⁻¹ or more; 500 s⁻¹ or more; 550 s⁻¹ or more; 600 s⁻¹ or more; 650 s⁻¹ or more; 700 s⁻¹ or more; 750 s⁻¹ or more; 800 s⁻¹ or more; 850 s⁻¹ or more; 900 s⁻¹ or more; 950 s⁻¹ or more; 1,000 s⁻¹ or more; 1,100 s⁻¹ or more; 1,200 s⁻¹ or more; 1,300 s⁻¹ or more; 1,400 s⁻¹ or more; 1,500 s⁻¹ or more; 1,600 s⁻¹ or more; 1,700 s⁻¹ or more; 1,800 s⁻¹ or more; 1,900 s⁻¹ or more; 2,000 s⁻¹ or more; 2,250 s⁻¹ or more; 2,500 s⁻¹ or more; 2,750 s⁻¹ or more; 3,000 s⁻¹ or more; 3,250 s⁻¹ or more; 3,500 s⁻¹ or more; 3,750 s⁻¹ or more; 4,000 s⁻¹ or more; 4,250 s⁻¹ or more; 4,500 s⁻¹ or more; 5,000 s⁻¹ or more; 5,500 s⁻¹ or more; 6,000 s⁻¹ or more; 6,500 s⁻¹ or more; 7,000 s⁻¹ or more; 7,500 s⁻¹ or more; 8,000 s⁻¹ or more; 8,500 s⁻¹ or more; 9,000 s⁻¹ or more; 9,500 s⁻¹ or more; 10,000 s⁻¹ or more; 11,000 s⁻¹ or more; 12,000 s⁻¹ or more; 13,000 s⁻¹ or more; 14,000 s⁻¹ or more; 15,000 s⁻¹ or more; 16,000 s⁻¹ or more; 17,000 s⁻¹ or more; 18,000 s⁻¹ or more; or 19,000 s⁻¹ or more) when subjected to multi-spin-echo measurements on a 1.5 T or 3 T MRI scanner.

In some examples, the coupling bath can have an R2 relaxation rate of 20,000 s⁻¹ or less (e.g., 19,000 s⁻¹ or less; 18,000 s⁻¹ or less; 17,000 s⁻¹ or less; 16,000 s⁻¹ or less; 15,000 s⁻¹ or less; 14,000 s⁻¹ or less; 13,000 s⁻¹ or less; 12,000 s⁻¹ or less; 11,000 s⁻¹ or less; 10,000 s⁻¹ or less; 9,500 s⁻¹ or less; 9,000 s⁻¹ or less; 8,500 s⁻¹ or less; 8,000 s⁻¹ or less; 7,500 s⁻¹ or less; 7,000 s⁻¹ or less; 6,500 s⁻¹ or less; 6,000 s⁻¹ or less; 5,500 s⁻¹ or less; 5,000 s⁻¹ or less; 4,500 s⁻¹ or less; 4,250 s⁻¹ or less; 4,000 s⁻¹ or less; 3,750 s⁻¹ or less; 3,500 s⁻¹ or less; 3,250 s⁻¹ or less; 3,000 s⁻¹ or less; 2,750 s⁻¹ or less; 2,500 s⁻¹ or less; 2,250 s⁻¹ or less; 2,000 s⁻¹ or less; 1,900 s⁻¹ or less; 1,800 s⁻¹ or less; 1,700 s⁻¹ or less; 1,600 s⁻¹ or less; 1,500 s⁻¹ or less; 1,400 s⁻¹ or less; 1,300 s⁻¹ or less; 1,200 s⁻¹ or less; 1,100 s⁻¹ or less; 1,000 s⁻¹ or less; 950 s⁻¹ or less; 900 s⁻¹ or less; 850 s⁻¹ or less; 800 s⁻¹ or less; 750 s⁻¹ or less; 700 s⁻¹ or less; 650 s⁻¹ or less; 600 s⁻¹ or less; 550 s⁻¹ or less; 500 s⁻¹ or less; 475 s⁻¹ or less; 450 s⁻¹ or less; 425 s⁻¹ or less; 400 s⁻¹ or less; 375 s⁻¹ or less; 350 s⁻¹ or less; 325 s⁻¹ or less; 300 s⁻¹ or less; 275 s⁻¹ or less; 250 s⁻¹ or less; 225 s⁻¹ or less; 200 s⁻¹ or less; 175 s⁻¹ or less; 150 s⁻¹ or less; 125 s⁻¹ or less; 100 s⁻¹ or less; 90 s⁻¹ or less; 80 s⁻¹ or less; 70 s⁻¹ or less; 60 s⁻¹ or less; 50 s⁻¹ or less; 45 s⁻¹ or less; 40 s⁻¹ or less; 35 s⁻¹ or less; or 30 s⁻¹ or less) when subjected to multi-spin-echo measurements on a 1.5 T or 3 T MRI scanner.

The R2 relaxation rate of the coupling bath can range from any of the minimum values described above to any of the maximum values described above. For example, the coupling bath can have an R2 relaxation rate of from 20 s⁻¹ to 20,000 s⁻¹ (e.g., from 20 s⁻¹ to 200 s⁻¹; from 200 s⁻¹ to 2,000 s⁻¹; from 2,000 s⁻¹ to 20,000 s⁻¹; from 50 s⁻¹ to 20,000 s⁻¹; or from 200 s⁻¹ to 20,000 s⁻¹) when subjected to multi-spin-echo measurements on a 1.5 T or 3 T MRI scanner.

In some examples, the coupling bath can have a T2 relaxation time of 50 milliseconds (ms) or less (e.g., 45 ms or less, 40 ms or less, 35 ms or less, 30 ms or less, 25 ms or less, 20 ms or less, 15 ms or less, 10 ms or less, 9 ms or less, 8 ms or less, 7 ms or less, 6 ms or less, 5 ms or less, 4.5 ms or less, 4 ms or less, 3.5 ms or less, 3 ms or less, 2.5 ms or less, 2 ms or less, 1.5 ms or less, 1 ms or less, 0.9 ms or less, 0.8 ms or less, 0.7 ms or less, 0.6 ms or less, 0.5 ms or less, 0.4 ms or less, 0.3 ms or less, 0.2 ms or less, 0.1 ms or less, 0.09 ms or less, 0.08 ms or less, 0.07 ms or less, 0.06 ms or less, 0.05 ms or less, 0.04 ms or less, 0.03 ms or less, or 0.02 ms or less) when subjected to multi-spin-echo measurements on a 1.5 T or 3 T MRI scanner. In some examples, the coupling bath can have a T2 relaxation time of 0.01 ms or more (e.g., 0.02 ms or more, 0.03 ms or more, 0.04 ms or more, 0.05 ms or more, 0.06 ms or more, 0.07 ms or more, 0.08 ms or more, 0.09 ms or more, 0.1 ms or more, 0.2 ms or more, 0.3 ms or more, 0.4 ms or more, 0.5 ms or more, 0.6 ms or more, 0.7 ms or more, 0.8 ms or more, 0.9 ms or more, 1 ms or more, 1.5 ms or more, 2 ms or more, 2.5 ms or more, 3 ms or more, 3.5 ms or more, 4 ms or more, 4.5 ms or more, 5 ms or more, 6 ms or more, 7 ms or more, 8 ms or more, 9 ms or more, 10 ms or more, 15 ms or more, 20 ms or more, 25 ms or more, 30 ms or more, 35 ms or more, or 40 ms or more) when subjected to multi-spin-echo measurements on a 1.5 T or 3 T MRI scanner. The T2 relaxation time of the coupling bath can range from any of the minimum values described above to any of the maximum values described above. For example, the coupling bath can have a T2 relaxation time of rom 0.01 ms to 50 ms (e.g., from 0.01 ms to 0.1 ms, from 0.1 ms to 1 ms, from 1 ms to 50 ms, from 0.01 ms to 40 ms, from 0.01 ms to 30 ms, from 0.01 ms to 10 ms, from 0.01 ms to 5 ms, from 0.05 ms to 50 ms, from 0.1 ms to 50 ms, from 0.5 ms to 50 ms, from 1 ms to 50 ms, or from 5 ms to 50 ms) when subjected to multi-spin-echo measurements on a 1.5 T or 3 T MRI scanner.

In some examples, the coupling bath has an MR signal in a T2 and/or T2*-weighted image that is less than the corresponding MR signal from a corresponding coupling bath in the absence of the plurality of paramagnetic particles, e.g. a coupling bath consisting essentially of or consisting of water (e.g., degassed water) by 50% or more (e.g., 55% or more, 60% or more, 65% or more, 70% or more, 75% or more, 80% or more, 85% or more, 90% or more, 95% or more, or 99% or more).

The coupling bath can, for example, have similar or the same mechanical, thermal, acoustic, and/or electromagnetic properties as a corresponding coupling bath in the absence of the plurality of paramagnetic particles, e.g. a coupling bath consisting essentially of or consisting of water (e.g., degassed water). In some examples, the coupling bath has a peak temperature change that is different than the corresponding peak temperature change for a corresponding coupling bath in the absence of the plurality of paramagnetic particles, e.g. a coupling bath consisting essentially of or consisting of water (e.g., degassed water), by 10% or less (e.g., 9% or less, 8% or less, 7% or less, 6% or less, 5% or less, 4% or less, 3% or less, 2% or less, or 1% or less) when insonated for 10 seconds at an acoustic power of from 50 to 1000 W (e.g., at an acoustic power to 50 W or more, 60 W or more, 70 W or more, 80 W or more, 90 W or more, 100 W or more, 125 W or more, 150 W or more, 175 W or more, 200 W or more, 250 W or more, 300 W or more, 350 W or more, 400 W or more, 450 W or more, 500 W or more, 600 W or more, 700 W or more, 800 W or more, or 900 W or more). In some examples, the coupling bath has a peak temperature change that is different than the corresponding peak temperature change for a corresponding coupling bath in the absence of the plurality of particles, e.g. a coupling bath consisting essentially of or consisting of water (e.g., degassed water), by 5° C. or less (e.g., 4.5° C. or less, 4° C. or less, 3.5° C. or less, 3° C. or less, 2.5° C. or less, 2° C. or less, 1.5° C. or less, 1° C. or less, or 0.5° C. or less), when insonated for 10 seconds at an acoustic power of from 50 to 1000 W.

In some examples, the MRI-guided procedure comprises MRI-guided microwave ablation, MRI-guided laser interstitial surgery, MRI-guided focused ultrasound (FUS), or a combination thereof. In some examples, the focused energy applied to the area of interest in the MRI-guided procedure comprises microwave energy, a laser, focused ultrasound (FUS), or a combination thereof.

In some examples, the focused energy applied to the area of interest in the MRI-guided procedure comprises focused ultrasound (FUS). In some examples, the focused energy applied to the area of interest in the MRI-guided procedure comprises focused ultrasound (FUS) and the area of interest of the subject comprises the abdomen, prostate, bone (e.g., skull), uterus, brain, or a combination thereof. In some examples, the MRI-guided procedure is a transcranial focused ultrasound (T-FUS) procedure. In certain examples, the coupling bath is acoustically compatible with clinical FUS procedures, such as transcranial FUS procedures. For example, the prefocal acoustic field used in the FUS procedure can be below the 0.5 cavitation probability threshold and/or below the non-trivial cavitation probability threshold of the coupling bath.

For example, coupling bath can have a probability of cavitation of 0.5 or less (e.g., 0.45 or less, 0.4 or less, 0.35 or less, 0.3 or less, 0.25 or less, 0.2 or less, 0.15 or less, 0.1 or less, or 0.05 or less) for a peak negative pressure of 30 mega pascals (MPa) or less (e.g., 25 MPa or less, 20 MPa or less, 15 MPa or less, 10 MPa or less, or 5 MPa or less) when subjected to highly focused, shocked, 5 cycle pulses at a pulse rate of 1 Hz.

In some examples, the coupling bath has a cavitation duty cycle of 0.5 or less (e.g., 0.45 or less, 0.4 or less, 0.35 or less, 0.3 or less, 0.25 or less, 0.2 or less, 0.15 or less, 0.1 or less, or 0.05 or less) for a peak negative pressure of 30 MPa or less (e.g., 25 MPa or less, 20 MPa or less, 15 MPa or less, 10 MPa or less, or 5 MPa or less) when subjected to continuous wave acoustic sonications with acoustic powers of from 1 to 1000 W (e.g., at an acoustic power of 5 W or more, 10 W or more, 15 W or more, 20 W or more, 25 W or more, 30 W or more, 35 W or more, 40 W or more, 45 W or more, 50 W or more, 60 W or more, 70 W or more, 80 W or more, 90 W or more, 100 W or more, 125 W or more, 150 W or more, 175 W or more, 200 W or more, 250 W or more, 300 W or more, 350 W or more, 400 W or more, 450 W or more, 500 W or more, 600 W or more, 700 W or more, 800 W or more, or 900 W or more) for 10 seconds.

In some examples, the size, shape, and/or composition of the plurality of paramagnetic particles; the concentration of the plurality of paramagnetic particles in the coupling bath; the presence or absence of the capping layer; the identity of the plurality of ligands when the capping layer is present; the average thickness of the capping layer when the capping layer is present; or a combination thereof can be selected such that the coupling bath has an r2 relaxivity of 20 per second per mM or more and/or an R2 relaxation rate of 20 per second or more when subjected to multi-spin-echo measurements on a 1.5 T or 3 T MRI scanner; the coupling bath has a T2 relaxation time of 50 ms or less when subjected to multi-spin-echo measurements on a 1.5 T or 3 T MRI scanner; the coupling bath has an MR signal in a T2 and/or T2*-weighted image that is less than the corresponding MR signal from a corresponding coupling bath in the absence of the plurality of paramagnetic particles, e.g. a coupling bath consisting essentially of or consisting of water (e.g., degassed water), by 50% or more; the coupling bath has similar or the same mechanical, thermal, acoustic, and/or electromagnetic properties as a coupling bath in the absence of the plurality of paramagnetic particles, e.g. a coupling bath consisting essentially of or consisting of water (e.g., degassed water); the coupling bath has a peak temperature change that is different than the corresponding peak temperature change for a corresponding coupling bath in the absence of the plurality of paramagnetic particles, e.g. a coupling bath consisting essentially of or consisting of water (e.g., degassed water), by 10% or less when insonated for 10 seconds at an acoustic power of from 50 to 1000 W; the coupling bath has a peak temperature change that is different than the corresponding peak temperature change for a corresponding coupling bath in the absence of the plurality of paramagnetic particles, e.g. a coupling bath consisting essentially of or consisting of water (e.g., degassed water), by 5° C. or less when insonated for 10 seconds at an acoustic power of from 50 to 1000 W; the coupling bath is acoustically compatible with clinical FUS procedures, such as transcranial FUS procedures; coupling bath has a probability of cavitation of 0.5 or less for a peak negative pressure of 30 MPa or less when subjected to highly focused, shocked, 5 cycle pulses at a pulse rate of 1 Hz; the coupling bath has a cavitation duty cycle of 0.5 or less for a peak negative pressure of 30 MPa or less when subjected to continuous wave acoustic sonications with acoustic powers of from 1 to 1000 W for 10 seconds; or a combination thereof.

Also disclosed herein are methods of making any of the coupling baths described herein. For example, the methods can comprise dispersing the plurality of paramagnetic particles in the water (e.g. degassed water). Dispersing the plurality of paramagnetic particles in the water (e.g., degassed water) can be accomplished by mechanical agitation, for example, mechanical stirring, shaking, vortexing, sonication (e.g., bath sonication, probe sonication, ultrasonication), and the like, or combinations thereof. In some examples, the methods can further comprise making the plurality of paramagnetic particles.

Also disclosed herein are methods of use and systems comprising any of the coupling baths described herein.

For example, also described herein are methods for a magnetic resonance imaging (MRI)-guided procedure on a subject wherein focused energy is applied to an area of interest of the subject, the method comprising providing any of the coupling baths described herein proximate the area of interest, such that, when magnetic resonance images are collected from the area of interest of the subject for the MRI guidance, the coupling bath reduces or prevents imaging artifacts in the magnetic resonance images for the MRI guidance. In some examples, the use of the coupling bath improves the image-guidance and/or safety of the MRI guided procedure relative to a corresponding coupling bath in the absence of the plurality of paramagnetic particles, e.g. a coupling bath consisting essentially of or consisting of water (e.g., degassed water). In some examples, the methods can further comprise collecting magnetic resonance images from the area of interest of the subject for the MRI guidance. The magnetic resonance images for the MRI guidance can, for example, be collected intraoperatively.

In some examples, the methods can further comprise circulating the coupling bath during the collection of the magnetic resonance images for the MRI guidance. Circulating the coupling bath can, for example, cool the subject, cool a portion of the MRI device and/or device for delivering the focused energy that is in contact with the coupling bath, minimize or translocate an acoustic nuclei if present, or a combination thereof.

In some examples, the MRI-guided procedure comprises MRI-guided microwave ablation, MRI-guided laser interstitial surgery, MRI-guided focused ultrasound (FUS), or a combination thereof. In some examples, the focused energy applied to the area of interest in the MRI-guided procedure comprises microwave energy, a laser, focused ultrasound (FUS), or a combination thereof.

In some examples, the focused energy applied to the area of interest in the MRI-guided procedure comprises focused ultrasound (FUS). In some examples, the focused energy applied to the area of interest in the MRI-guided procedure comprises focused ultrasound (FUS) and the area of interest of the subject comprises the abdomen, prostate, bone (e.g., skull), uterus, brain, or a combination thereof. In some examples, the MRI-guided procedure is a transcranial focused ultrasound (T-FUS) procedure. In certain examples, the coupling bath is acoustically compatible with clinical FUS procedures, such as transcranial FUS procedures. For example, the prefocal acoustic field used in the FUS procedure can be below the 0.5 cavitation probability threshold and/or below the non-trivial cavitation probability threshold of the coupling bath.

Also disclosed herein are systems for a magnetic resonance imaging (MRI)-guided procedure on a subject wherein focused energy is applied to an area of interest of the subject, the system comprising any of the coupling baths described herein proximate the area of interest and a magnetic resonance imaging device configured to collect images from the area of interest of the subject for the MRI guidance, wherein when the magnetic resonance images are collected from the area of interest of the subject for MRI guidance, the coupling bath reduces or prevents imaging artifacts in the magnetic resonance images for the MRI guidance.

In some examples, the systems can further comprise a device configured to apply focused energy to the area of interest in the subject. For example, the device configured to apply focused energy to the area of interest in the subject can comprise a transcranial focused ultrasound (T-FUS) device. The T-FUS device can, for example, comprise a transducer and the coupling bath can be located between the transducer and the area of interest of the subject.

In some examples, the systems can further comprise a means for circulating the coupling bath (e.g., a pump), such as during the collection of the magnetic resonance images for the MRI guidance. Circulating the coupling bath can, for example, cool the subject, cool a portion of the MRI device and/or device for delivering the focused energy that is in contact with the coupling bath, minimize or translocate an acoustic nuclei if present, or a combination thereof.

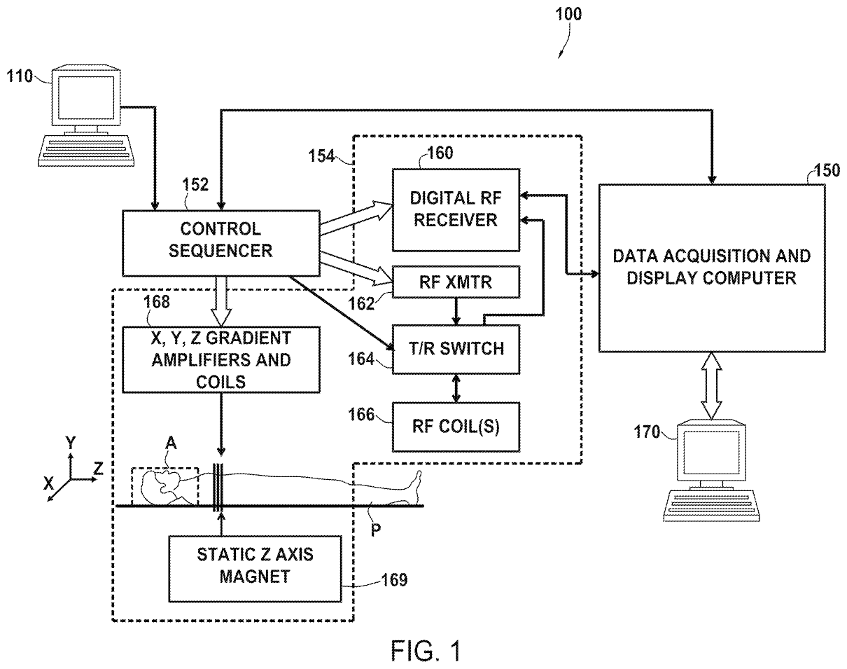

FIG. 1 is a system diagram illustrating an imaging system capable of implementing aspects of the present disclosure in accordance with one or more example embodiments. FIG. 1 illustrates an example of a magnetic resonance imaging (MRI) system 100, including a data acquisition and display computer 150 coupled to an operator console 110, an MRI real-time control sequencer 152, and an MRI subsystem 154. The MRI subsystem 154 may include XYZ magnetic gradient coils and associated amplifiers 168, a static Z-axis magnet 169, a digital RF transmitter 162, a digital RF receiver 160, a transmit/receive switch 164, and RF coil(s) 166. The MRI subsystem 154 may be controlled in real time by control sequencer 152 to generate magnetic and radio frequency fields that stimulate magnetic resonance phenomena in a subject P to be imaged, for example to implement magnetic resonance imaging sequences in accordance with various embodiments of the present disclosure. Reconstructed images, such as contrast-enhanced image(s) of an area of interest A of the subject P may be shown on display 170.

The area of interest A shown in the example embodiment of FIG. 1 corresponds to a head region of subject P, but it should be appreciated that the area of interest for purposes of implementing various aspects of the disclosure presented herein is not limited to the head area. It should be recognized and appreciated that the area of interest in various embodiments may encompass various areas of subject P associated with various physiological characteristics, such as, but not limited to the head and brain region, chest region, heart region, abdomen, upper or lower extremities, or other organs or tissues. Various aspects of the present disclosure are described herein as being implemented on portions of the skeletal system of human subjects.

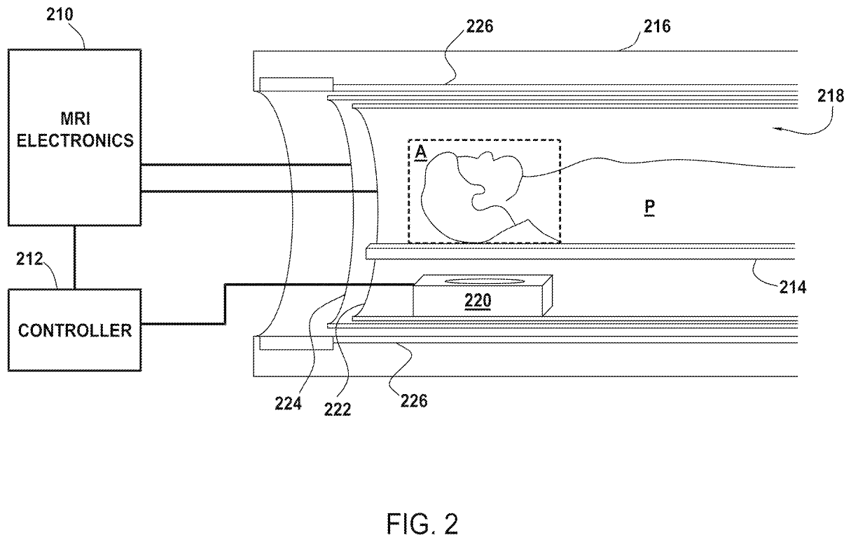

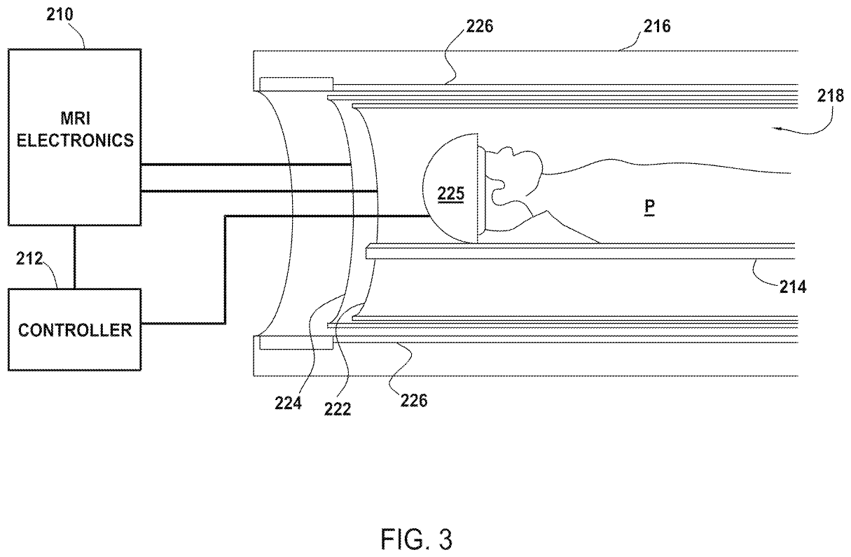

It should be appreciated that any number and type of computer-based medical imaging systems or components, including various types of commercially available medical imaging systems and components, may be used to practice certain aspects of the present disclosure. Systems as described herein with respect to imaging are not intended to be specifically limited to the particular system shown in FIG. 1. Likewise, systems as described herein with respect to the application of localized energy are not intended to be specifically limited to the particular systems shown in FIG. 2 and FIG. 3 described below.

One or more data acquisition or data collection steps as described herein in accordance with one or more embodiments may include acquiring, collecting, receiving, or otherwise obtaining data such as imaging data corresponding to an area of interest. By way of example, data acquisition or collection may include acquiring data via a data acquisition device, receiving data from an on-site or off-site data acquisition device or from another data collection, storage, or processing device. Similarly, data acquisition or data collection devices of a system in accordance with one or more embodiments of the present disclosure may include any device configured to acquire, collect, or otherwise obtain data, or to receive data from a data acquisition device within the system, an independent data acquisition device located on-site or off-site, or another data collection, storage, or processing device.

FIG. 2 and FIG. 3 are schematic diagrams showing two respective embodiments of systems with focused ultrasound (FUS) used with MRI, each of which is capable of implementing aspects of the present disclosure in accordance with one or more embodiments. FIG. 2 shows a first type of FUS device 220 used in combination with MRI. The MRI system may comprise one or more components of the system 100 shown in FIG. 1. As shown, RF coils 222, gradient coils 224, static Z axis magnet 226, and magnetic housing 216 surround the patient P when the patient is positioned on the table 214 inside of the MRI bore 218. A controller 212 communicates with MRI system electronics 210 as well as the FUS device (220 in FIG. 2, 225 in FIG. 3). The MRI system electronics 210 can include one or more components of the MRI subsystem 154 shown in FIG. 1. A user computer (not shown) may communicate with the controller 212 for control of the MRI system and FUS device functions.

As shown in the embodiment of FIG. 2, a first type of FUS device 220 is disposed under the head of the patient P and within the bore 218 such that focused ultrasound energy may be applied to target the area of interest A.

In FIG. 3, a second type of FUS device 225 surrounds the patient's head, as may be used for thermal therapy applied to tissues of or near the brain. The device 225 may have multiple ultrasound transducers for applying focused energy to particular target areas of interest of the head of the patient.

The devices 220 and/or 225 can be configured to apply localized energy to a targeted region within the area of interest A which includes tissues of or near the brain. The devices 220 and/or 225 can include the coupling bath, e.g., the localized energy can be transmitted through the coupling bath to the targeted region within the area of interest A. The MRI components of the system (including MRI electronics 210) are configured to work within a larger MRI system to acquire magnetic resonance data and for reconstructing images of all or regions of the area of interest.

Control of the application of the focused energy via the controller 212 may be managed by an operator using an operator console (e.g., user computer). The controller 212 (which, as shown is also coupled to MRI electronics 210) may also be configured to manage functions for the application and/or receiving of MR signals. For example, the controller 212 may be coupled to a control sequencer such as the control sequencer 152 shown in FIG. 1.

Although the FUS devices 220, 225 shown in the embodiments of FIG. 2 and FIG. 3 utilize ultrasound transducer(s) as the source for delivering localized energy to an area of interest, it should be appreciated that other types of devices may alternatively be used without departing from the patentable scope of the present disclosure. Other possible types of treatment/application devices that may be utilized include, but are not limited to, laser and/or RF ablation devices.

In some examples, disclosed herein are systems, methods, and computer readable mediums for an acoustic coupling bath using magnetite nanoparticles for MR-guided transcranial focused ultrasound surgery. In some examples, disclosed herein are systems, methods, and computer readable mediums for an acoustic coupling media to improve image-guidance and treatment safety in transcranial focused ultrasound procedures. It should be appreciated that a variety of ultrasound related systems and methods may be utilized as part of implementing or practicing aspects of the various embodiments of the present invention.

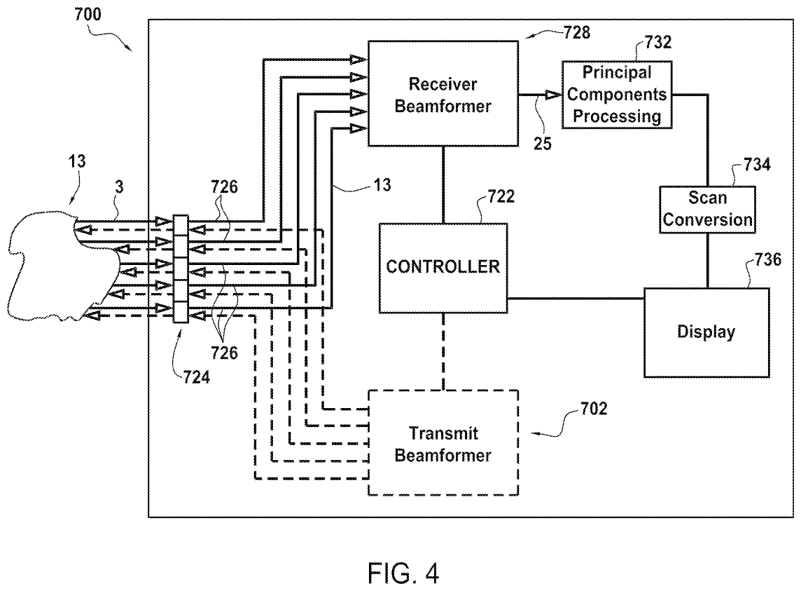

FIG. 4 is a basic, schematic representation of an ultrasound system 700 according to an aspect of an embodiment of the present invention that is referred to in order to generally describe the operations of an ultrasound system to produce an image of an object 13. System 700 may optionally include a transmit beamformer 702 which may include input thereto by controller 722 to send electrical instructions to array 724 as to the specifics of the ultrasonic waves to be emitted by array 724. Alternatively, system 700 may be a receive only system and the emitted waves may be directed to the object 13 from an external source.

In either case, echoes 3 reflected by the object 13 (and surrounding environment) are received by array 724 and converted to electrical (e.g., radio frequency (RF)) signals 726 that are input to receive beamformer 728. Controller 722 may be external of the beamformer 728, as shown, or integrated therewith. Controller 722 automatically and dynamically changes the distances at which scan lines are performed (when a transmit beamformer 702 is included) and automatically and dynamically controls the receive beamformer 728 to receive signal data for scan lines at predetermined distances. Distance/depth is typically calculated assuming a constant speed of sound in tissue (e.g., 1540 m/s or as desired or required) and then time of flight is recorded such that the returning echoes have a known origination. The summed RF lines output by the receive beamformer 728 are input to a principal components processing module 732, which may be separate from and controlled by, or incorporated in controller 722.

The assembled output may be input into a scan converter module 734. The image formed within the scan converter 734 is displayed on display 736. Although FIG. 4 has been described as an ultrasound system, it is noted that transducers 724 may alternatively be transducers for converting electrical energy to forms of energy other than ultrasound and vice versa, including, but not limited to radio waves (e.g., where system 700 is configured for RADAR), visible light, infrared, ultraviolet, and/or other forms of sonic energy waves, including, but not limited to SONAR, or some other arbitrary signal of arbitrary dimensions greater than one (such as, for example, a signal that is emitted by a target).

The concept of applying an acoustic coupling bath using magnetite nanoparticles for MR guided transcranial focused ultrasound surgery may be implemented and utilized with one or more computing devices 240.

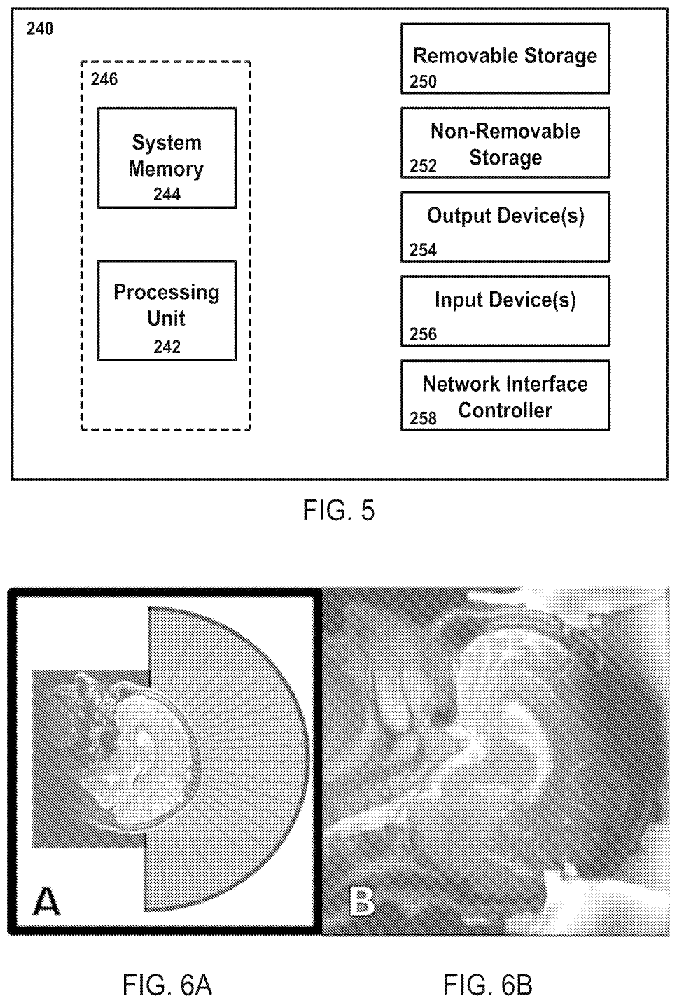

In some examples, the systems can further comprise a computing device. FIG. 5 illustrates an example computing device 240 upon which examples disclosed herein may be implemented. The computing device 240 can include a bus or other communication mechanism for communicating information among various components of the computing device 240. In its most basic configuration, computing device 240 typically includes at least one processing unit 242 (a processor) and system memory 244. Depending on the exact configuration and type of computing device, system memory 244 may be volatile (such as random access memory (RAM)), non-volatile (such as read-only memory (ROM), flash memory, etc.), or some combination of the two. This most basic configuration is illustrated in FIG. 5 by a dashed line 246. The processing unit 242 may be a standard programmable processor that performs arithmetic and logic operations necessary for operation of the computing device 240.

The computing device 240 can have additional features/functionality. For example, computing device 240 may include additional storage such as removable storage 250 and non-removable storage 252 including, but not limited to, magnetic or optical disks or tapes. The computing device 240 can also contain network connection(s) 258 that allow the device to communicate with other devices. The computing device 240 can also have input device(s) 256 such as a keyboard, mouse, touch screen, antenna or other systems configured to communicate with the camera in the system described above, etc. Output device(s) 254 such as a display, speakers, printer, etc. may also be included. The additional devices can be connected to the bus in order to facilitate communication of data among the components of the computing device 240.

The processing unit 242 can be configured to execute program code encoded in tangible, computer-readable media. Computer-readable media refers to any media that is capable of providing data that causes the computing device 240 (i.e., a machine) to operate in a particular fashion. Various computer-readable media can be utilized to provide instructions to the processing unit 242 for execution. Common forms of computer-readable media include, for example, magnetic media, optical media, physical media, memory chips or cartridges, a carrier wave, or any other medium from which a computer can read. Example computer-readable media can include, but is not limited to, volatile media, non-volatile media and transmission media. Volatile and non-volatile media can be implemented in any method or technology for storage of information such as computer readable instructions, data structures, program modules or other data and common forms are discussed in detail below. Transmission media can include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 242 can execute program code stored in the system memory 244. For example, the bus can carry data to the system memory 244, from which the processing unit 242 receives and executes instructions. The data received by the system memory 244 can optionally be stored on the removable storage 250 or the non-removable storage 252 before or after execution by the processing unit 242.

The computing device 240 typically includes a variety of computer-readable media. Computer-readable media can be any available media that can be accessed by device 240 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 244, removable storage 250, and non-removable storage 252 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 240. Any such computer storage media can be part of computing device 240.

It should be understood that the various techniques described herein can be implemented in connection with hardware or software or, where appropriate, with a combination thereof. Thus, the methods, systems, and associated signal processing of the presently disclosed subject matter, or certain aspects or portions thereof, can take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs can implement or utilize the processes described in connection with the presently disclosed subject matter, e.g., through the use of an application programming interface (API), reusable controls, or the like. Such programs can be implemented in a high level procedural or object-oriented programming language to communicate with a computer system. However, the program(s) can be implemented in assembly or machine language, if desired. In any case, the language can be a compiled or interpreted language and it may be combined with hardware implementations.

The analysis of signals can be carried out in whole or in part on one or more computing device. For example, the system may comprise one or more additional computing device.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Acoustic Coupling Bath Using Magnetite Nanoparticles for MR-Guided Transcranial Focused Ultrasound Surgery

Abstract

Purpose: Acoustic coupling baths, nominally composed of degassed water, play important roles during transcranial focused ultrasound surgery. However, this large water bolus also degrades the quality of intraoperative magnetic resonance (MR) guidance imaging. In this study, the feasibility of using dilute, aqueous magnetite nanoparticle suspensions to suppress these image degradations while preserving acoustic compatibility were tested. The effects of these suspensions on metrics of image quality and acoustic compatibility were examined for two types transcranial focused ultrasound insonation regimes: low duty cycle histotripsy procedures and high duty cycle thermal ablation procedures.

Methods: MR guidance imaging was used to monitor thermal ablations of in vitro gel targets using a coupling bath composed of various concentrations of aqueous, suspended, magnetite nanoparticles in a clinical transcranial transducer under stationary and flowing conditions. Thermal deposition was monitored using MR thermometry simultaneous to insonation. Then, using normal degassed water as a coupling bath, various concentrations of aqueous, suspended, magnetite nanoparticles were placed at the center of this same transducer and insonated using high duty cycle pulsing parameters. Passive cavitation detectors recorded cavitation emissions, which were then used to estimate the relative number of cavitation events per insonation (cavitation duty cycle) and cavitation dose estimates of each nanoparticle concentration. Finally, the nanoparticle mixtures were exposed to low duty cycle, histotripsy pulses. Passive cavitation detectors monitored cavitation emissions, which were used to estimate cavitation threshold pressures.

Results: The nanoparticles reduced the MR signal of the coupling bath by 90% in T2 and T2*-weighted images and also removed almost all imaging artifacts caused by coupling bath motion. The coupling baths caused less than 5% changes in peak temperature change achieved during sonication, as observed via MR thermometry. At low insonation duty cycles, the nanoparticles decreased the cavitation threshold pressure by about 15±7% in a manner uncorrelated with nanoparticle concentration. At high insonation duty cycles, the 0.5 cavitation duty cycle acoustic power threshold varied linearly with nanoparticle concentration.

Conclusions: Dilute aqueous magnetite nanoparticle suspensions effectively reduced MR imaging artifacts caused by the acoustic coupling bath. They also attenuated acoustic power deposition by less than 5%. For low duty cycle insonation regimes, the nanoparticles decreased the cavitation threshold by 15±7%. However, for high duty cycle regimes, the nanoparticles decreased the threshold for cavitation in proportion to nanoparticle concentration.

Introduction

Transcranial focused ultrasound (T-FUS) is a platform technology that promises noninvasive neurosurgical therapies, including thermal [1] and mechanical ablations [2], for a host of indications [3-5]. The technology's main advantages include its non-invasive nature and spatial selectivity [4]. Because T-FUS is noninvasive, surgeons rely on real-time image guidance to ensure acceptable treatment outcomes. Magnetic resonance imaging (MRI) can provide anatomical imaging, thermometry, functional assessments, and lesion identification and classification [6] and is routinely used to guide T-FUS procedures. When the quality and accuracy of MR imaging increases, the surgeon's degree of control over treatment safety and efficacy also increases [7,8].

T-FUS procedures use a large array of electronically steered acoustic transducer elements to control the deposition of acoustic energy into the brain [9]. These elements sit on a rigid, hemispherical frame whose surface, due to geometrical constraints, stands several centimeters off of the patient's scalp. To improve acoustic transmittance, protect the patient's scalp against surface burns, and minimize the occurrence of unwanted cavitation, clinical implementations of T-FUS circulate this space with a chilled degassed coupling bath [10]. Degassed water remains the most commonly used coupling bath because it is inexpensive, readily conveys heat, possesses a similar acoustic impedance to soft tissues, and does not easily absorb or shock the acoustic pulses as they propagate.

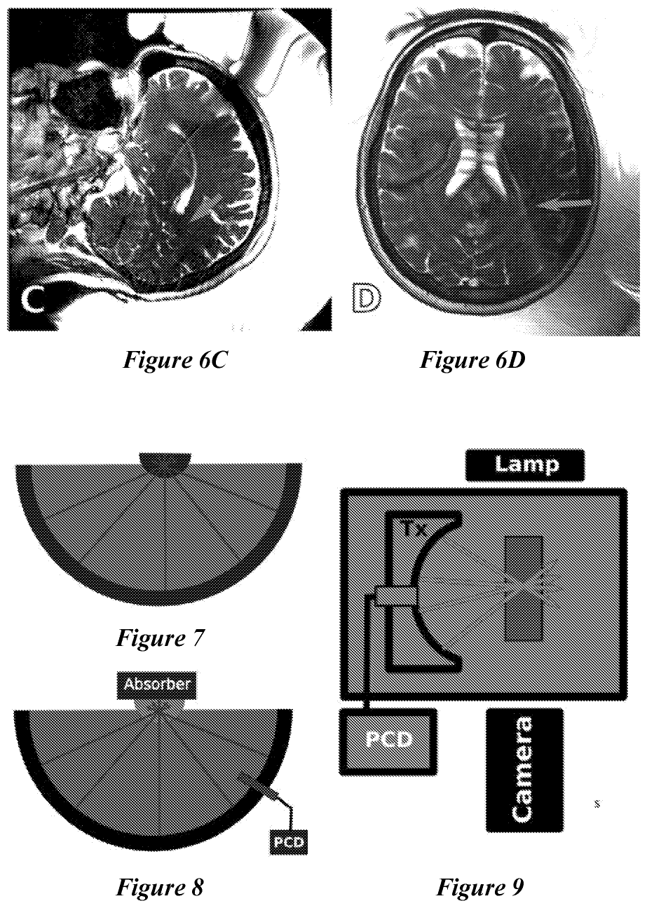

While vital to acoustic transmission, this bath also contributes to imaging errors during intraoperative MR scanning. For example, the coupling bath requires a large imaging field of view, thereby increasing image acquisition times and limiting the maximum sampling rate of a thermometry time course. The bath's large relative signal magnitude can skew the MR scanner's prescan algorithm to favor image quality in the coupling bath. Poor calibration leads to blurred or shifted images and degraded signal levels and tissue contrasts in the patient [11]. Meanwhile, continuous circulation and degassing would decrease heating in the patient's scalp and skull and may also reduce the chance of unwanted cavitation in the coupling bath. However, it is currently impossible to cool and degas the coupling bath while using MRI to monitor a sonication because water movement produces data inconsistencies across MRI's piecewise image sampling process, resulting in imaging errors. Examples of these imaging artifacts are displayed in FIG. 6B-FIG. 6D. A coupling bath that could remove these sources of imaging error and also enable continuous cooling and degassing during sonication would improve the reliability and accuracy of MR guidance imaging during T-FUS procedures.

In an effort to remove similar artifacts associated with a focused ultrasound device dedicated to breast ablations, Deckers et al. [12] and Merckel et al. [13] dissolved manganese chloride into a partitioned subsection of the coupling bath volume immediately in contact with the patient. The manganese accelerated the coupling bath's transverse relaxation process, rendering it, when sampled at late echo times, too small relative to the patient's MR signal to produce major imaging artifacts. The device also employed localized imaging coils to reduce image sensitivity to the manganese-free partition of the water bath. While effective for the dedicated breast device, dissolved manganese chloride remains suboptimal for use in T-FUS because the size of the transducer forces one to use receiver coils that are sensitive to the entire volume of the coupling bath. Filling the entire bath with dissolved salts would expose the transducer surface to corrosive materials, reduce the electrical isolation between the patient from the transducer hardware, and considering the 7-12 L volume of the water bath, introduce a non-trivial source of image noise.

Herein, replacing manganese chloride with aqueous suspensions of superparamagnetic magnetite nanoparticles is proposed. Previous work has shown that particles of encapsulated magnetite efficiently accelerate the MR transverse relaxation process [14-16]. They can also be made electrically neutral, chemically stable [17], and biocompatible [18], and their high relaxivity permits very dilute concentrations. For example, an aqueous, 1 mM magnetite concentration suspension should accelerate the coupling bath's R₂ rate from its native 0.3 s⁻¹ to 100 s⁻¹ while only altering its density, and thus its acoustic impedance, by 0.02%. It is hypothesized that, when used as an acoustic coupling medium during a T-FUS procedure, these suspensions will simultaneously preserve acoustic coupling and rapidly decay the bath's MR signal, rendering it too small relative to the patient's MR signal to produce major imaging artifacts and allow continuous circulation. Further, unlike other candidate coupling baths—such as deuterated water, perfluoropolyethers and oils, and chelated contrast agents, —magnetite is abundant, inexpensive, and, at dilute concentrations, does not alter acoustic attenuation, fluid viscosity, heat convection, or fluid conductivity.

Two remaining important acoustic compatibility aspects are the chance that the nanoparticles will enhance acoustic cavitation or heating in the coupling bath. It is widely known that impurities promote cavitation activity in water [19,20]. Enhanced acoustic cavitation in the coupling bath would be undesirable because it can block acoustic transmission, damage the surfaces of the transducer and the scalp, and falsely signal that cavitation has occurred within the patient. Meanwhile, enhanced heating in the water bath or on the patient's skin would promote skin burns. Several previous reports have examined potential acoustic interactions between magnetite nanoparticles and focused ultrasound. Work by Smith et al. [21] and Ho et al. [22] found that aqueous, aggregated magnetite particles seed cavitation nuclei upon insonation. Meanwhile, Sun et al. [23] and Wang et al. [24] found that injected, surface-modified magnetite particles enhanced the efficacy of continuous wave HIFU treatments in ex vivo liver and rabbit and in vivo murine cancer models, which was attributed to enhanced acoustic absorption. However, it is not possible to use these studies to predict the prevalence of nanoparticle-enhanced cavitation or heating in the desired application. For example, Smith et al. [21], Sun et al. [23], and Wang et al. [24] were not able to report the concentration of the magnetite nanoparticles at the insonation site. Meanwhile, Ho et al. [22] insonated nanoparticle concentration of 116 mM, which is two orders of magnitude larger than necessary for the desired purposes herein. Smith et al. [21] and Ho et al. [22] used acoustic pulsing duty cycles of 0.5-50%, which differ from the 100% and <0.1% duty cycles associated with T-FUS thermal and mechanical ablations (histotripsy), respectively. Finally, Sun et al. [23] and Wang et al. [24] did not quantify cavitation activity, which does not preclude the possibility of cavitation-enhanced heating [25].

In this study, the feasibility of using dilute, aqueous magnetite nanoparticle suspensions as an acoustic coupling bath for T-FUS was examined. First, the ability of these nanoparticles to suppress the MR signal of the coupling bath and alleviate motion artifacts on MR guidance imaging during an in vitro T-FUS procedure was examined. Then their impact on heat deposition in an in vitro gel target was examined. Finally, their impact on cavitation threshold metrics under T-FUS thermal (100% duty cycle) and mechanical (<0.1% duty cycle) ablation insonation schemes were examined.

Materials and Methods

Magnetite Nanoparticles and Characterization

Aqueous, magnetite nanoparticles with an advertised 99.5% purity were purchased from a commercial vendor (Stock #US7568, US Research Nanomaterials, Houston, Tex.). Nanoparticle size was determined with dynamic light scattering (DLS). Briefly, the purchased nanoparticles were diluted in deionized water (18 megaohm resistivity) and then sonicated for 30 minutes in an ultrasonic water bath (Model #15337410, Fisher Scientific, Suwanee, Ga.). DLS measurements were then performed at 25° C. using a Malvern Zetasizer Nano-ZS and the Malvern Zetasizer Software (v7.12). While the particle size was advertised to be 20 nm, dynamic light scattering revealed that the particles agglomerated in water with a mean intensity-average diameter of 240 nm, a similar value to that reported in Smith et al. [21] and Ho et al [22]. The particle size distribution measured by DLS was monomodal with a polydispersity index ˜0.4 and did not change with sonication time up to 30 minutes. The advertised concentration of 20% w/wt was assumed to be correct and all material was assumed to have a Fe₃O₄ chemical composition. The particles demonstrated an R2 relaxivity of 526 s⁻¹ mM⁻¹ when examined using standard multi-spin-echo measurements on a 3 T MRI scanner (MR 750, General Electric, Waukesha, Wis.) which is much larger than the value of 93 s⁻¹ mM⁻¹ reported for a commercial magnetite-based contrast medium (Feridex/Endorem) in water at 37° C. and 3 T [26]. In the experiments described below, various coupling baths were formed by diluting the purchased nanoparticles in degassed water. Due to aggregation, the particles would begin settling within 20 minutes after suspension and care was taken to either continuously circulate or periodically re-suspend the solutions over the course of each experiment.

### Experiment 1: MR Imaging

To test the ability of the particles to suppress the coupling bath's MR signal, a 30 cm, 1024 element, 650 kHz transducer array (Insightec Exablate Neuro, Haifa Israel), was oriented “face up” with its bowl concave toward the ceiling and filled with aqueous nanoparticle concentrations ranging from 0-0.49 mM. A small polyacrylamide gel phantom was placed at the transducer focus and scanned using anatomical T2-weighted sequences and thermometry sequences common to T-FUS procedures. The thermometry scans were performed using a single-slice, multi-echo gradient-echo sequence (GRE, repetition time: 28 ms, field of view: 28 cm, matrix size: 256×128 pixels, bandwidth: 35.7 kHz, echo times: 3.3, 8.0, 12.8, 17.6, and 22.4 ms, frame rate: 3.5 s). The T2-weighted sequences were either a 2D, multi-slice, half-Fourier acquisition, single shot, turbo-spin-echo sequence (HASTE, repetition time: 4.6 s, echo time: 81 ms, echo train length: 88, field of view: 24×24 cm, matrix size: 384×160 pixels, slice thickness: 8 mm, bandwidth: 325 kHz) or a 2D, multi-slice, turbo-spin echo sequence (TSE, repetition time: 7.7 s, echo time: 81 ms, echo train length: 24, field of view: 32 cm, matrix size: 256×256 pixels, slice thickness: 3 mm, bandwidth: 195 kHz). To prevent particle settling, the water was continuously circulated using a small pump for all acquisitions except for one turbo-spin echo image acquired with a stationary 0 mM nanoparticle concentration coupling bath. This experimental setup is depicted schematically in FIG. 7.

### Experiment 2: Thermal Deposition

In this experiment, the ability of the particles, as a coupling bath, to attenuate thermal deposition of ultrasound in a target gel was tested. The 30 cm, 1024 element transducer used in Experiment 1 was oriented “face up” and its bowl was filled aqueous nanoparticle concentrations of 0 or 0.25 mM. A small, hemispherical, plastic holder (acoustic attenuation less than 0.18 dB, volume of 250 mL) was placed at the transducer focus. This holder was filled with a gel with an acoustic absorbance of less than 0.0012 Np mm⁻¹ (Aquaflex, Parker Laboratories, Fairfield, N.J.) and repeatedly insonated for 10 s at acoustic powers ranging from 50 to 600 W. Insonations at each power level were repeated three times with approximately four minute delays between each instance to allow for sample cooling. The GRE MR thermometry sequence described in Experiment 1 was used, simultaneous to insonation, to monitor heating in the gel target. To prevent settling, the coupling baths with nanoparticle concentrations greater than 0 mM were continuously circulated using a pump and hosing apparatus. This experimental setup is depicted schematically in FIG. 7.

### Experiment 3: Cavitation Threshold During High Duty Cycle Insonation

The transducer used in experiments one and two was again oriented “face up” and filled with degassed water. A small, hemispherical, polystyrene shell with 1 mm wall-thickness (acoustic attenuation less than 0.18 dB, volume of 115 mL) was placed at the focus and filled with aqueous magnetite nanoparticles ranging in concentration from 0 to 0.5 mM. An acoustic absorber (Aptflex F48, Precision Acoustics, Dorchester, UK) was then placed on top of the plastic holder to both attenuate acoustic reflections at the air-fluid interface and to prevent surface vibrations. The transducer then deposited 10 s duration, continuous wave sonications with acoustic powers ranging from 1 to 1000 W into the holder. Because all sonications could be completed in less than 5 minutes and excess sediment was not observed at the bottom of the holder after each experiment, the particles were not periodically perturbed or resuspended after initial mixture. This experiment is depicted schematically in FIG. 8.

Cavitation detection was accomplished using eight wideband passive cavitation detectors (PCD) built into the transducer device. These detectors sampled the ambient acoustic emissions at a rate of 2 MHz for a 10 ms period and employed a passive, low-pass filter with a −6 dB cutoff frequency near 500 kHz. The system continuously collected data, producing approximately 1020 spectra per 10 s insonation. The Fourier transforms of the sampled emissions were then exported to a personal computer and analyzed using custom routines written in MATLAB (The MathWorks, Natick, Mass.). Specifically, spectra from all eight receivers (which were very similar in magnitude and shape) were first averaged together and the root mean square average (RMSA) of the 60-500 kHz band of each spectra was computed. Baseline spectral behavior was computed by taking the mean and standard deviation of the RMSA estimates from the 1 W insonation of the 0 mM nanoparticle concentration sample. During all other insonations, a cavitation event was assumed to occur when the RMSA value of a spectrum exceeded this baseline mean by more than 5 standard deviations. The cavitation duty cycle was then computed as the fraction of spectra acquired during an insonation that satisfied this criterion. The resulting curves were then fit to a Gaussian cumulative distribution function. Finally, the inertial cavitation dose of a given sonication was estimated by summing together the RMSA estimates associated with each insonation [27]. For comparison with the low-duty cycle regime, the 0.5 cavitation duty cycle power thresholds returned by the fit were converted to pressure (P) using the formulation P=\sqrt{Pₐz/A}, where Pₐ is the acoustic power, z is the acoustic impedance of water (1.48*106 Rayls), and A is the transducer's focal area (2.545 mm²), as supplied by the manufacturer.

### Experiment 4: Cavitation Threshold During Low Duty cycle Insonation

A 700 kHz, shock-scattering histotripsy transducer [28] (Histosonics, Ann Arbor, Mich.) was used to examine the propensity of the particles to seed cavitation under a low duty cycle insonation regime. The transducer consisted of 36 elements arranged in a 13 cm aperture and an 11 cm focal distance. Aqueous magnetite particles with concentrations of 0-0.36 mM were then used to fill a custom, water-proof chamber designed with both an acoustically transparent window as well as optical windows to allow high speed videography. The center of the chamber was placed at the transducer focus and the entire setup was immersed in a tank of degassed, circulating water. The transducer then deposited 100 highly focused, shocked, 5 cycle pulses into the chamber at a pulse rate of 1 Hz (duty cycle of 0.0007%). The window attenuated the acoustic pulses by −0.13 dB. This experiment was repeated as the peak negative pressures of the acoustic pulses ranged from −8 to −42 MPa, which encompassed the range of pressures over which cavitation activity was observed. To prevent settling, the chamber was gently rotated after every other instance where the acoustic pressure was altered, a period of approximately every 5 minutes. One of the 36 elements was connected to an oscilloscope through a cable splitter and a 100× attenuator and used as a PCD system in a similar manner as has been reported elsewhere [29, 30]. This experiment is depicted schematically in FIG. 9.

Because the opacity of the coupling bath varied with nanoparticle concentration, optical images could not be reliably used to detect cavitation events over all experiments reported here. Instead, the camera was used to calibrate the PCD system in the following manner. First, the oscilloscope was triggered to only record voltage fluctuations across the transducer element at times corresponding to acoustic propagation across twice the transducer's focal distance. Then, with the chamber filled with degassed water, the negative pressures of the acoustic pulses were increased until cavitation events could be consistently detected on optical photographs. The voltage threshold used to determine a cavitation event while during insonation of any coupling bath was then taken to be 80% of the peak recorded voltage on the oscilloscope after averaging over 4 calibration experiments.

For each nanoparticle concentration, the fractions of PCD voltages above the detection threshold were computed and fitted, as a function of peak negative pressure and via a least-squares algorithm, to a Gaussian cumulative distribution in the same manner as published elsewhere [31, 32]. This fitted curve was then taken to express the relative probability of a cavitation event as a function of exposure to a pulse at a given peak negative pressure.

Results

### Experiment 1: MR Imaging Results

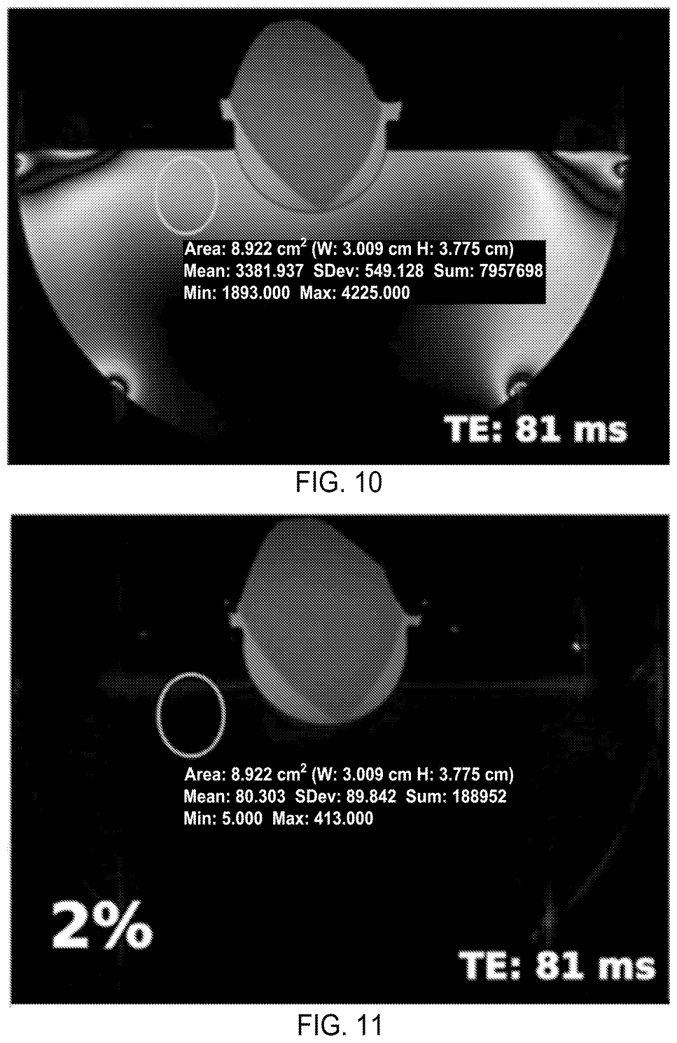

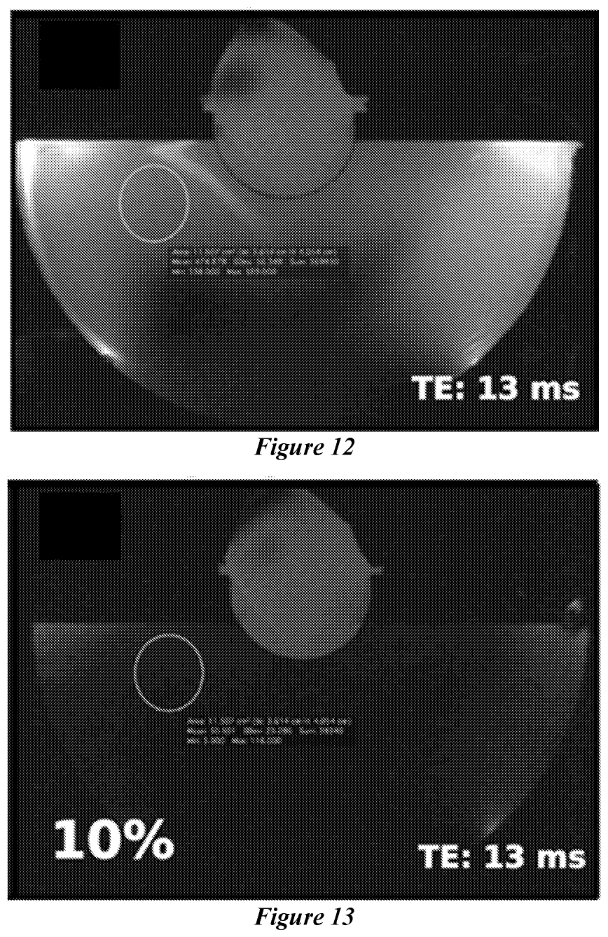

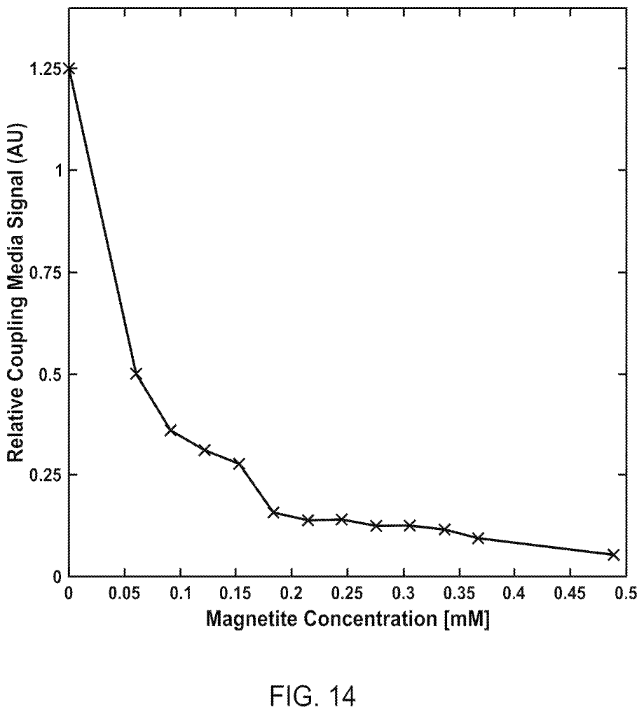

Example images of a gel target acquired with the fast-spin-echo and gradient-echo sequence are displayed in FIG. 10-FIG. 13 for two different nanoparticle concentrations within the coupling bath. At the echo-times listed in the figure, a 0.25 mM nanoparticle concentration was able to suppress 90% of the coupling bath relative to degassed water. FIG. 14 plots the magnitude of the coupling bath signal relative to that of the gel target as a function of particle concentration. While no attempt at fitting has been made, the signal magnitude roughly follows a multi-exponential decay pattern. Further, it was observed that the largest magnitude of signal suppression occurs at the jump between 0 and 0.6 mM nanoparticle concentrations, with decreasing step sizes as nanoparticle concentration increases. The exact nature of this curve can vary with the echo spacing used to make the measurement.

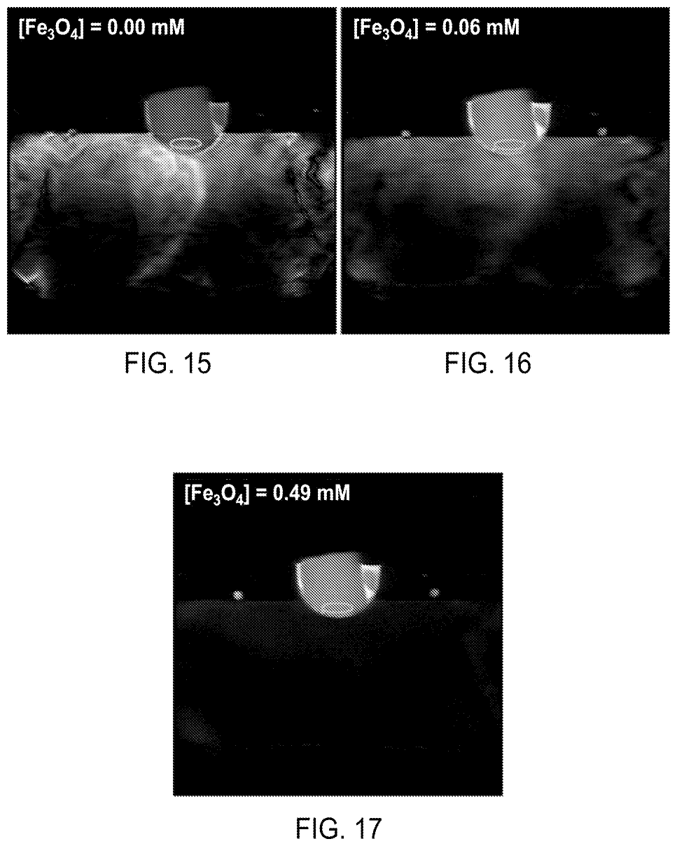

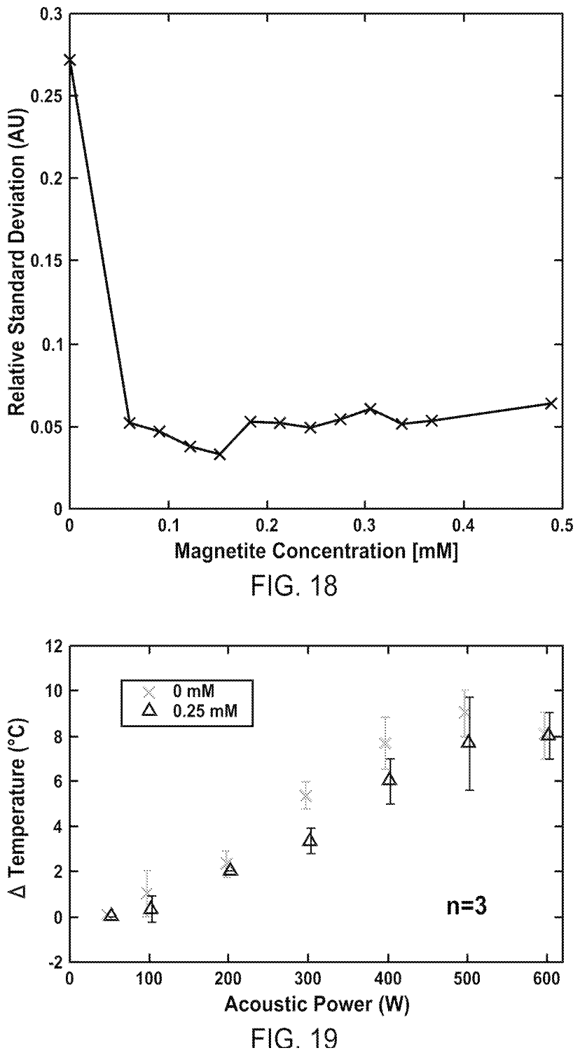

Example images from the HASTE sequences are displayed in FIG. 15-FIG. 17 for three different nanoparticle concentrations within the coupling bath. These images display motion artifacts in both the coupling bath itself and the gel target that reduce with nanoparticle concentration. FIG. 18 also plots the standard deviation of an ROI centered on the portion of the gel target subjected to visible motion artifact, normalized by the average signal intensity of the portion of the gel not subject to visible motion artifact. At 0 mM nanoparticle concentration, the standard deviation of the signal in the ROI is 30% that of the mean gel signal intensity. Higher nanoparticle concentrations reduce this parameter to 5% of the mean gel signal intensity. Similar to that shown in FIG. 14, the majority of the motion artifact is removed with the smallest tested nanoparticle concentration.

### Experiment 2: Thermal Deposition Results

The mean and standard deviation of the maximum change in temperature (averaged over a 9-voxel square window centered on the hottest voxel) achieved in the gel phantom across all 3 sonications are plotted in FIG. 19 as a function of acoustic power and nanoparticle concentration. At a given power and coupling bath composition, the average temperature increase varied by 2-3° C.—a value that is larger than the 1° C. precision of the thermometry sequence. On average, sonications transmitting through the 0.25 mM nanoparticle concentration coupling bath achieved 1-3 degree lower peak temperature increase than identical sonications using a water coupling bath. Finally, while not shown in a figure, PCD traces reported enhanced RMSA levels during sonications with acoustic powers greater than 50 W when using the 0.25 mM concentration coupling bath. This phenomenon was not observed while using the 0 mM coupling bath.

### Experiment 3: Cavitation Threshold During High Duty Cycle Insonation Results

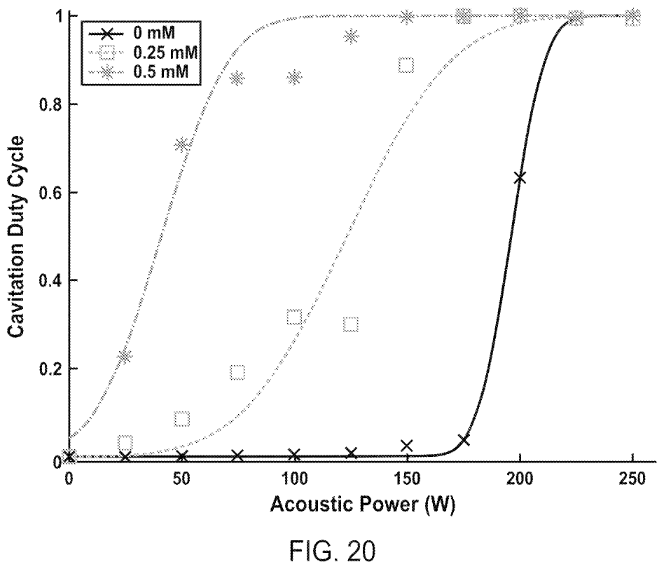

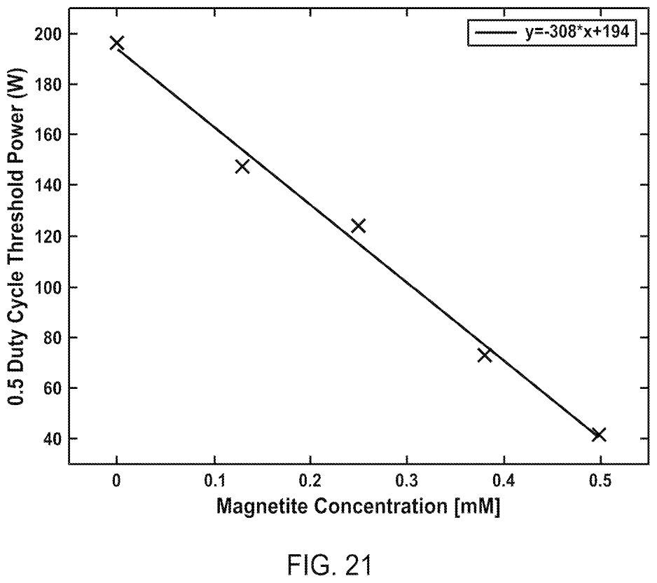

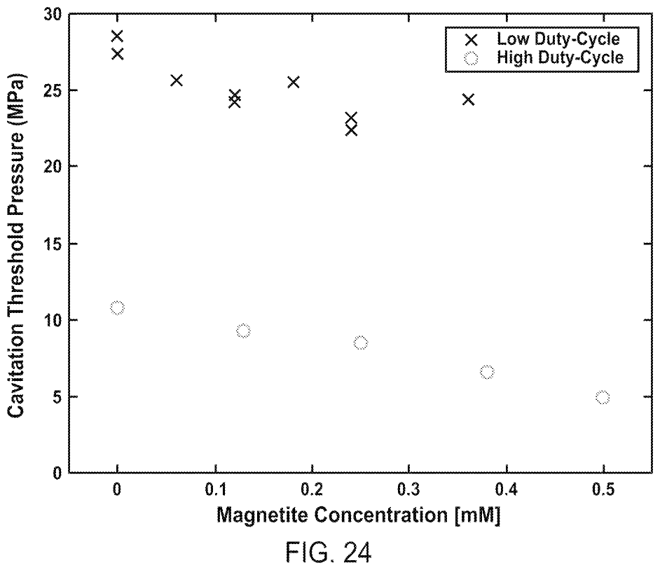

Example curves of the cavitation duty cycle are plotted, along with least-squares fits to a Gaussian cumulative distribution function, in FIG. 20 as a function of acoustic power and nanoparticle concentration. All nanoparticle solutions demonstrated a monotonically increasing cavitation duty cycle and are well described by the fitting function. Nanoparticle concentrations greater than 0 mM shifted these curves toward lower acoustic powers. This behavior is most compactly expressed by the 0.5 duty cycle parameter returned by the fitting algorithm and is plotted as a function of nanoparticle concentration in FIG. 21. The 0.5 threshold in this case decreases linearly with acoustic power with a least-squares-derived slope of 308 WmM⁻¹ nanoparticle concentration. These same thresholds, converted to pressure, are displayed in FIG. 24.

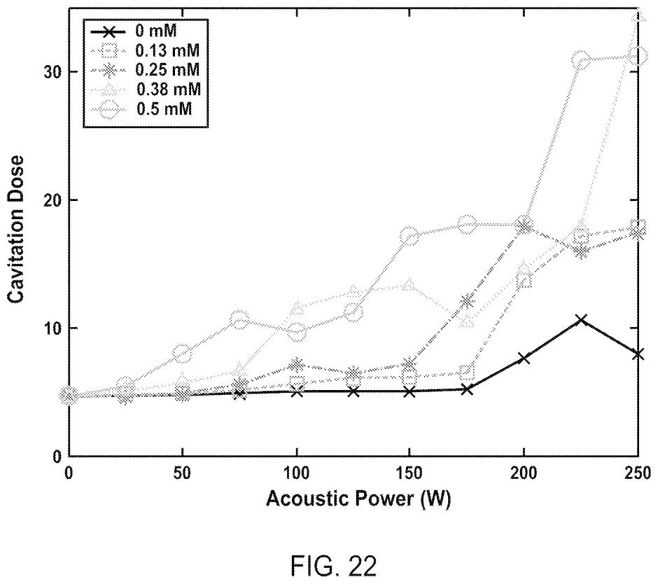

The temporal distribution of spectra satisfying the cavitation criterion appeared to mimic the behavior of a binomial random process. For example, at acoustic powers well below the 0.5 threshold, the few spectra satisfying the cavitation criterion appeared sprinkled randomly in time. As the acoustic power increased or the nanoparticle concentration increased, the relative proportion of spectra detecting a cavitation event also increased but continued to follow no obvious temporal pattern. Finally, it was observed, in instances where a cavitation event was detected, that the relative magnitude of the corresponding RMSA increased with nanoparticle concentration. This effect can be observed in FIG. 22, which plots the estimated cavitation dose of each sonication as a function of acoustic power and nanoparticle concentration.

### Experiment 4: Cavitation Threshold During Low Duty Cycle Insonation Results

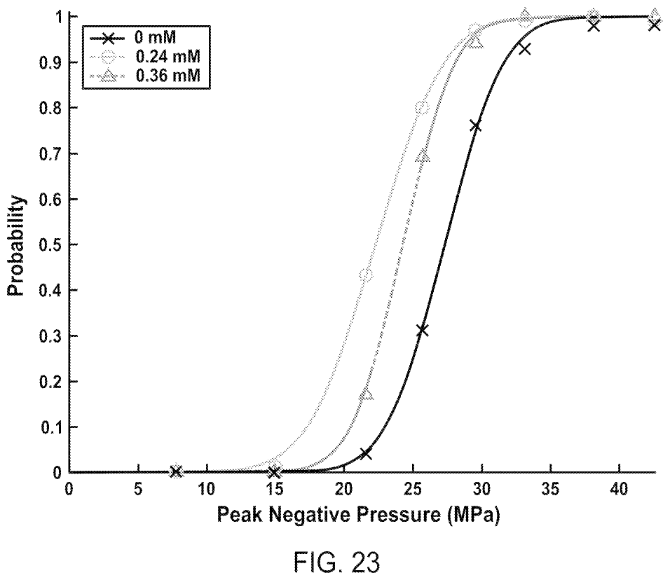

Example plots of the observed fraction of acoustic pulses that produced detected cavitation events, along with least-squares fits to a Gaussian cumulative distribution function, are shown in FIG. 23. All nanoparticle solutions demonstrated monotonically increasing cavitation probabilities as a function of peak negative pressure, with distinct transitions from a 0% cavitation fraction to a 100% cavitation fraction observed at peak negative pressure levels between −18 and −30 MPa. All data sets were well described by the Gaussian cumulative distribution function. Nanoparticle concentrations greater than 0 mM shifted these curves toward lower negative pressure values. This behavior is most compactly expressed by the 0.5 cavitation probability parameter returned by the fitting algorithm and is plotted, as a function of nanoparticle concentration, in FIG. 24. For comparison, the power thresholds in FIG. 21 are converted to acoustic pressure and also displayed. The nanoparticles decrease this threshold by an average and range of 15±7%. Unlike the results from Experiment 3, these thresholds displayed too much variability to confidently determine a mathematical relationship with pressure. Finally, the smallest magnitude peak negative pressure that produced a detectable cavitation event was −22 MPa for all tested concentrations (including 0 mM) except 0.24 mM—which demonstrated a smallest magnitude pressure of −15 MPa.

Discussion

The experimental study presented above examines the feasibility of using dilute, aqueous suspensions of commercially available magnetite nanoparticles as a means to reduce the degradative effects of the acoustic coupling bath on MR guidance imaging during T-FUS procedures. The experiments tested the acoustic compatibility of these suspensions at likely T-FUS insonation frequencies (650 and 750 kHz) and their impact on MR guidance imaging while sonicating a gel phantom. Experiment 1 demonstrated the ability of the nanoparticles to both reduce the signal magnitude of the coupling bath and remove associated motion artifacts for a broad range of dilute nanoparticle concentrations. Experiment 2 showed that a dilute nanoparticle concentration coupling bath imparted less than 5% loss in thermal deposition over a broad range of acoustic powers and also promoted detectable cavitation activity. Experiment 3 demonstrated that, under a high duty cycle, continuous wave insonation regime, the particles reduce the 0.5 cavitation duty cycle power threshold by 308 W per each mM of nanoparticle concentration. Finally, experiment 4 indicated that these particles reduce the 0.5 probability cavitation pressure threshold when exposed to a low duty cycle, histotripsy insonation regime. Variability in the estimated pressure thresholds prevented us from confidently determining a mathematical relationship with pressure.

The main concern over the use of aqueous magnetite nanoparticles as a coupling bath is that the particles will enhance losses as an acoustic wave propagates. Losses via direct scatter off the particles, impedance mismatch, and absorption can be minimized by suspending as few particles as possible within the coupling bath. However, widespread cavitation throughout the prefocal field can also potentially block the propagation of acoustic energy into a desired target. In addition, in a clinical setting, cavitation emissions can act as important safety signals and be used to prevent tissue damage. Therefore, controlling cavitation in the prefocal acoustic field should be an important design consideration for magnetite infused acoustic coupling bath. In the case of low duty cycle insonations used in histotripsy, large negative pressures at the focus are primarily obtained by geometric focal gains using a hemispherical transducer and the acoustic pressures in the coupling bath can be expected to remain small compared to the cavitation threshold pressures observed in experiment 4. However, if treatment targets lie near the skull [33], the prefocal field in the coupling bath may approach that at the focus in order to surmount acoustic losses in the bone and cavitation in the coupling bath can become quite likely. For these cases, future coupling bath designs should consider the possibility of very large pressure fields within the bath.

In the case of high duty cycle thermal ablation procedures, care must be taken that the power deposition in the prefocal field does not approach the 0.5 cavitation duty cycle threshold. This relationship will place limitations on the acceptable maximum nanoparticle concentration for a given sonication power level. The temperature measurements described in Experiment 2 indicate that, in this system, the prefocal field remained sufficiently below the 120 W threshold associated with a 0.25 mM concentration coupling bath to impede thermal deposition by no more that 5%. It is entirely possible that higher nanoparticle concentrations will induce more spatially and temporally abundant cavitation and more seriously impede thermal buildup in the target. One limitation of this study is its lack of thermal measurements over a broader range of particle concentrations.

A second concern over using magnetite nanoparticles is settling. So long as the nanoparticle concentration is sufficiently large to remove motion artifacts, settling can easily be avoided by continuously circulating the water bath during scanning. However, it is possible that some applications, such as a histotripsy ablation near the skull surface, require a low nanoparticle concentration, which may lead to residual water motion artifacts. In these cases, it will be desirable to have the water bath stationary during MR imaging. If the imaging time lasts longer than 5 minutes, particle settling may begin to produce spatially varying suppression of the coupling bath or sufficiently increase the concentration of particles to induce high rates of cavitation. It may be possible to mitigate these effects by intermittently perturbing the water bath over the course of imaging.

A future direction of study is indicated by Smith et al. [21], who found that coating magnetite nanoparticles with silica can increase the cavitation threshold pressure. Surface modifications can be an intriguing method to reduce the chance of cavitation activity during continuous-wave sonications. However, they can also reduce the relaxivity of magnetite particles, reducing their ability to suppress the MR signal of the coupling bath [16]. Another future direction of study would be using cavitation deletion pulses to coalesce or dissolve potential nuclei [34] among the nanoparticles either before or after mixture with degassed water.

While there are a number of alternative methods to suppress the MR signal of the coupling bath, including chelated contrast agents, inversion recovery MR pulse sequences, and spatially selective RF pulses, the use of magnetite particles remains particularly attractive for several reasons. First, the rate of signal loss from the coupling bath, for a fixed echo time and repetition rate, is maximized at low nanoparticle concentrations. This relationship ensures that even very dilute solutions can suppress a large amount of imaging artifacts, better preserving, relative to chelated contrast agents, the favorable mechanical, acoustic, thermal, and electrical properties of water as a coupling bath. Second, the performance of the nanoparticles is invariant with large magnetic field and RF inhomogeneities that would reduce the efficacy of spatially-selective RF pulses. Third, while the magnetite nanoparticles will not prove beneficial to images acquired with very short echo times, they remain compatible with a wider variety of acquisition parameters and techniques than inversion recovery techniques, including rapid, gradient-echo sequences commonly used for thermometry.

Under some circumstances, it is desirable to observe the coupling bath on MRI. For example, to prevent unwanted cavitation or heating, it is important to deactivate transducer elements whose beam path intersects air pockets such as bubbles lodged on the patient's scalp or folds in the rubber membrane used to prevent the coupling bath from spilling out. In nominal practice, the bright coupling bath observed on a T2-weighted scan can nicely delineate these features. However, using the proposed nanoparticle-infused coupling bath would eliminate strong T2-weighted contrast between an air pocket and the coupling bath and prevent easy detection. A potential solution could be to use short TE scans during this phase of treatment preparation to capture the coupling bath prior to decay.

One limitation of this study is that it does not cover the full range of acoustic frequencies that may be used in T-FUS procedures. Acoustic absorption and scatter can vary with insonation frequency [35]. Studies across a spectrum of frequencies can better characterize the nature and origin of any absorption or scatter caused by the nanoparticles. A second limitation is that the delays between insonations in Experiment 3 are too short to allow complete cooling, which likely has contributed to large heating variations at several acoustic powers. A more rigorous analysis of the sound speed, attenuation, and impedance of the coupling media is can more accurately predict expected temperature losses while using a nanoparticle-infused coupling bath. This study also does not examine the effect of the magnetite nanoparticles on the T1 of the coupling bath nor does it examine imaging benefits gained by using T1-weighted imaging techniques, which are an important class of neuroimaging methods.

Conclusion

This study examines the feasibility of using dilute, aqueous suspensions of commercially available magnetite nanoparticles as an acoustic coupling bath during transcranial focused ultrasound surgeries. The results show that, at dilute concentrations, these nanoparticles can effectively reduce MR signal from the coupling bath for a broad class of T2- and T2*-weighted scans without causing serious losses in heating during sonication. Meanwhile, experiments using both high and low duty cycle insonation regimes demonstrated that the particles can reduce cavitation thresholds by at least 15%. Under a high duty cycle regime, this reduction is particularly pronounced and scales linearly with particle concentration. These results suggest that magnetite nanoparticle suspensions are a feasible means to resolve MR image artifacts induced by the acoustic coupling bath during transcranial focused ultrasound surgeries so long as the prefocal acoustic field and nanoparticle concentration can be constrained below the appropriate cavitation threshold.

### REFERENCES

The following patents, applications and publications as listed below and throughout this document are hereby incorporated by reference in their entirety herein.
1. Elias W J, Lipsman N, Ondo W G, et al. A Randomized Trial of Focused Ultrasound Thalamotomy for Essential Tremor. N Engl J Med. 2016; 375(8):730-739. doi:10.1056/NEJMoa1600159
2. Sukovich J R, Cain C A, Pandey A S, et al. In vivo histotripsy brain treatment. J Neurosurg. October 2018:1-8. doi:10.3171/2018.4.JNS172652
3. Ghanouni P, Pauly K B, Elias W J, et al. Transcranial MRI-Guided Focused Ultrasound: A Review of the Technologic and Neurologic Applications. Am J Roentgenol. 2015; 205(1):150-159. doi:10.2214/AJR.14.13632
4. Ellis S, Rieke V, Kohi M, Westphalen A C. Clinical applications for magnetic resonance guided high intensity focused ultrasound (MRgHIFU): Present and future. J Med Imaging Radiat Oncol. 2013; 57(4):391-399. doi:10.1111/1754-9485.12085
5. Colen R R, Sahnoune I, Weinberg J S. Neurosurgical Applications of High-Intensity Focused Ultrasound with Magnetic Resonance Thermometry. Neurosurg Clin N Am. 2017; 28(4):559-567. doi:10.1016/j.nec.2017.05.008
6. Hectors S J C G, Jacobs I, Moonen C T W, Strijkers G J, Nicolay K. MRI methods for the evaluation of high intensity focused ultrasound tumor treatment: Current status and future needs. Magn Reson Med. 2016; 75(1):302-317. doi:10.1002/mrm.25758
7. Hectors S J C G C G, Jacobs I, Moonen C T W W, Strijkers G J, Nicolay K. MRI methods for the evaluation of high intensity focused ultrasound tumor treatment: Current status and future needs. Magn Reson Med. 2015; 317(1):n/a-n/a. doi:10.1002/mrm.25758
8. Ghanouni P, Pauly K B, Elias W J, et al. Transcranial MRI-guided focused ultrasound: A review of the technologic and neurologic applications. Am J Roentgenol. 2015; 205(1):150-159. doi:10.2214/AJR.14.13632
9. Hynynen K, Jones R M. Image-guided ultrasound phased arrays are a disruptive technology for non-invasive therapy. Phys Med Biol.:1-50. doi:10.1088/0031-9155/61/17/R206
10. Chang W S, Jung H H, Kweon E J, Zadicario E, Rachmilevitch I, Chang J W. Unilateral magnetic resonance guided focused ultrasound thalamotomy for essential tremor: practices and clinicoradiological outcomes. J Neurol Neurosurg Psychiatry. 2015; 86(3):257-264. doi:10.1136/jnnp-2014-307642
11. Odéen, Henrik; Patil, Sunil; Bolster, Bradley; Bhat Himanshu; Parker D L. Evaluation and tradeoffs of 2D and 3D Cartesian MR temperature imaging (MRTI) for brain applications. In: 6th International Symposium on Focused Ultrasound. Reston, Va.; 2018:BR-46.
12. Deckers R, Merckel L G, Denis de Senneville B, et al. Performance analysis of a dedicated breast MR-HIFU system for tumor ablation in breast cancer patients. Phys Med Biol. 2015; 60(14):5527-5542. doi:10.1088/0031-9155/60/14/5527
13. Merckel L G, Knuttel F M, Deckers R, et al. First clinical experience with a dedicated MRI-guided high-intensity focused ultrasound system for breast cancer ablation. Eur Radiol. 2016; 26(11):4037-4046. doi:10.1007/s00330-016-4222-9
14. Gillis P, Koenig SH. Transverse relaxation of solvent protons induced by magnetized spheres: application to ferritin, erythrocytes, and magnetite. Magn Reson Med. 1987; 5(4):323-345. doi:10.1002/mrm.1910050404
15. Lee N, Hyeon T. Designed synthesis of uniformly sized iron oxide nanoparticles for efficient magnetic resonance imaging contrast agents. Chem Soc Rev. 2012; 41(7):2575-2589. doi:10.1039/c1cs15248c
16. Carroll M R J, Huffstetler P P, Miles W C, et al. The effect of polymer coatings on proton transverse relaxivities of aqueous suspensions of magnetic nanoparticles. Nanotechnology. 2011. doi:10.1088/0957-4484/22/32/325702
17. Goff J D, Huffstetler P P, Miles W C, et al. Novel phosphonate-functional poly(ethylene oxide)-magnetite nanoparticles form stable colloidal dispersions in phosphate-buffered saline. Chem Mater. 2009; 21(20):4784-4795. doi:10.1021/cm901006g
18. Pothayee N, Balasubramaniam S, Pothayee N, et al. Magnetic nanoclusters with hydrophilic spacing for dual drug delivery and sensitive magnetic resonance imaging. J Mater Chem B. 2013. doi:10.1039/c2tb00275b
19. Apfel R E. The Role of Impurities in Cavitation-Threshold Determination. J Acoust Soc Am. 1970; 48(5B):1179-1186. doi:10.1121/1.1912258
20. Crum L A. Tensile strength of water. Nature. 1979; 278(5700):148-149. doi:10.1038/278148a0
21. Smith M J, Ho V H B, Darton N J, Slater N K H. Effect of Magnetite Nanoparticle Agglomerates on Ultrasound Induced Inertial Cavitation. Ultrasound Med Biol. 2009; 35(6):1010-1014. doi:10.1016/j.ultrasmedbio.2008.12.010
22. Ho V H B, Smith M J, Slater N K H. Effect of magnetite nanoparticle agglomerates on the destruction of tumor spheroids using high intensity focused ultrasound. Ultrasound Med Biol. 2011; 37(1):169-175. doi:10.1016/j.ultrasmedbio.2010.09.007
23. Sun Y, Zheng Y, Ran H, et al. Superparamagnetic PLGA-iron oxide microcapsules for dual-modality US/MR imaging and high intensity focused US breast cancer ablation. Biomaterials. 2012; 33(24):5854-5864. doi:10.1016/j.biomaterials.2012.04.062
24. Wang Z, Qiao R, Tang N, et al. Active targeting theranostic iron oxide nanoparticles for MRI and magnetic resonance-guided focused ultrasound ablation of lung cancer. Biomaterials. 2017; 127:25-35. doi:10.1016/j.biomaterials.2017.02.037
25. Sokka S D, King R, Hynynen K. MRI-guided gas bubble enhanced ultrasound heating in in vivo rabbit thigh. Phys Med Biol. 2003; 48(2):223-241. doi:10.1088/0031-9155/48/2/306
26. Rohrer M, Bauer H, Mintorovitch J, Requardt M, Weinmann H-JJ. Comparison of magnetic properties of MRI contrast media solutions at different magnetic field strengths. Invest Radiol. 2005; 40(11):715-724. doi:10.1097/01.r1i.0000184756.66360.d3
27. Chen W-S, Brayman A A, Matula T J, Crum L A. Inertial cavitation dose and hemolysis produced in vitro with or without Optison. Ultrasound Med Biol. 2003; 29(5):725-737. doi:10.1016/S0301-5629(03)00013-9
28. Roberts W W, Teofilovic D, Jahnke R C, Patri J, Risdahl J M, Bertolina J A. Histotripsy of the prostate using a commercial system in a canine model. J Urol. 2014; 191(3):860-865. doi:10.1016/j.juro.2013.08.077
29. Allen S P, Hernandez-Garcia L, Cain C A, Hall T L. MR-based detection of individual histotripsy bubble clouds formed in tissues and phantoms. Magn Reson Med. 2016; 76(5):1486-1493. doi:10.1002/mrm.26062
30. Vlaisavljevich E, Lin K-W, Warnez M T, et al. Effects of tissue stiffness, ultrasound frequency, and pressure on histotripsy-induced cavitation bubble behavior. Phys Med Biol. 2015; 60(6):2271-2292. doi:10.1088/0031-9155/60/6/2271
31. Maxwell A D, Cain C A, Hall T L, Fowlkes J B, Xu Z. Probability of cavitation for single ultrasound pulses applied to tissues and tissue-mimicking materials. Ultrasound Med Biol. 2013; 39(3):449-465. doi:http://dx.doi.org/10.1016/j.ultrasmedbio.2012.09.004
32. Vlaisavljevich E, Gerhardson T, Hall T, Xu Z. Effects of f-number on the histotripsy intrinsic threshold and cavitation bubble cloud behavior. Phys Med Biol. 2017; 62(4):1269-1290. doi:10.1088/1361-6560/aa54c7
33. Sukovich J, Xu Z, Kim Y, et al. Targeted Lesion Generation Through the Skull Without Aberration Correction Using Histotripsy. IEEE Trans Ultrason Ferroelectr Freq Control. 2016; 63(5):671-682. doi:10.1109/TUFFC.2016.2531504
34. Duryea A P, Cain C A, Tamaddoni H A, Roberts W W, Hall T L. Removal of residual nuclei following a cavitation event using low-amplitude ultrasound. IEEE Trans Ultrason Ferroelectr Freq Control. 2014; 61(10):1619-1626. doi:10.1109/TUFFC.2014.006316
35. Holmes A K, Challis R E, Wedlock D J. A Wide Bandwidth Study of Ultrasound Velocity and Attenuation in Suspensions: Comparison of Theory with Experimental Measurements. J Colloid Interface Sci. 1993; 156(2):261-268. doi:10.1006/jcis.1993.1109

### Example 2

Essential tremor (ET) is a debilitating movement disorder with a prevalence as high as 4% of the population. Approximately 50% of ET patients do not respond to medication. Many are also wary of the risks associated with conventional surgical therapies, such as deep brain stimulation and gamma knife. Meanwhile, transcranial focused ultrasound (T-FUS) offers immediate, non-invasive relief from ET symptoms by employing an array of ultrasound transducers to selectively heat and destroy a small thalamic nucleus, disrupting the neural pathways that cause ET. The procedure precludes stitches, sedation, or hospitalization, thereby reducing patient morbidities and treatment costs. Because it is noninvasive, T-FUS relies on accurate magnetic resonance imaging (MRI) data to estimate both thalamic location and ultrasound-induced heating.

However, the coupling water bath that conducts ultrasound from the transducer to the patient also introduces MR estimation error by (1) introducing water flow and (2) imposing a large imaging field-of-view. Flowing water causes data inconsistencies across MRI's piecewise image acquisition process, corrupting the estimates derived from MR images. Meanwhile, the large diameter of the water bath limits the acquisition speed and resolution of MR images because of the tradeoffs needed to avoid superimposing signal from the bath onto the more critical signal from the patient. A method that can remove these water-bath-induced MRI data errors would significantly improve the safety and precision of T-FUS.

In this study, an acoustic coupling media is developed that can remove coupling-bath-induced MR data errors, enabling a smaller imaging field-of-view and removing flow-induced inconsistences. This media contains acoustically compatible magnetite nanoparticles that can rapidly decay the bath's MR signal, rendering it small relative to that of the patient and unable to degrade MRI data. This coupling media can minimize flow-induced and field-of-view-induced MR estimation errors. This study comprises the following specific aims:

Aim 1—Synthesize magnetite nanoparticles to suppress the water bath's MR signal. Colloidal, magnetite nanoparticles enveloped in a hydrophilic polymeric coating are developed. The magnetite can accelerate the media's transverse relaxation rate to ˜100 s⁻¹, rendering it undetectable by most MR scans. The hydrophilic coating can improve the particles' acoustic compatibility.

Aim 2—Assess acoustic cavitation threshold pressures. The most salient property governing the acoustic compatibility of the nanoparticles developed in Aim 1, the cavitation threshold pressure, is measured. The impact various concentrations of these particles have on this threshold is measured using two acoustic pulsing schemes pertinent to T-FUS.

Aim 3—In situ demonstration of improved MR data fidelity. The impact of these nanoparticles on relevant measures of MRI data fidelity during T-FUS treatments of in situ tissue phantoms is measured while under water flow and reduced imaging field-of-view conditions.

Impact

T-FUS has already garnered FDA approval for the treatment of ET and tremor-dominant Parkinson's disease. Further, T-FUS world leaders are currently conducting clinical trials on the treatment of neuropathic pain. This study aims to design, develop, and pursue further translation of a method that will improve MRI data accuracy during T-FUS.

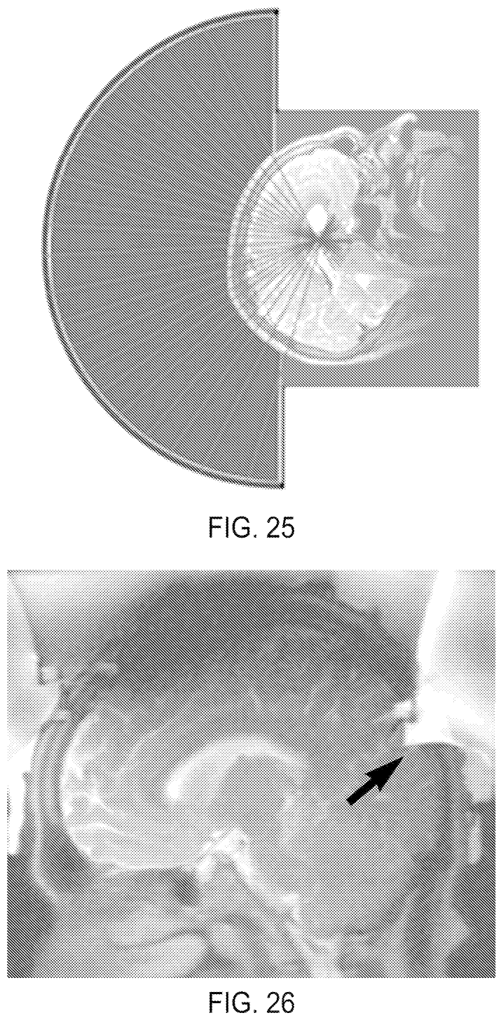

T-FUS works by transmitting ultrasound energy from an array of distributed sound sources through an acoustically conducting water bath, through the patient's scalp, skull, and dura matter, and, finally, into the target in the brain (i.e., sonication). The water bath is essential to surgery because it couples sound transmission between the transducer and the patient (FIG. 25). Meanwhile, because T-FUS is noninvasive, surgeons rely on real-time image guidance to ensure acceptable treatment outcomes. Magnetic resonance imaging (MRI) is well suited to this task by providing anatomical imaging thermometry, functional assessments, and lesion identification and classification [X1]. However, when the quality and accuracy of MR imaging decreases, the surgeon's degree of control over treatment safety and efficacy also diminishes [X2, X3].

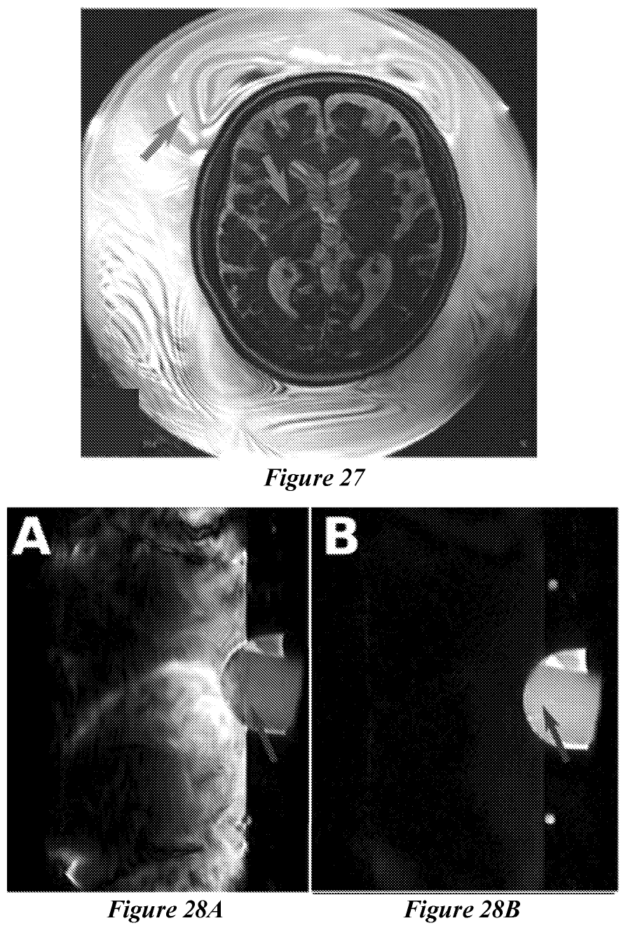

The coupling water bath present during T-FUS procedures causes significant imaging data errors during intraoperative MR scanning. For example, the presence of the water bath requires a large imaging field of view, thereby increasing image acquisition times and limiting the maximum sampling rate of a thermometry time course (FIG. 26). Water movement due to circulation and vibrations in the water bath vibration produce data inconsistencies across MRI's piecewise image sampling process, causing further imaging errors (FIG. 27). A technique that removes these sources of imaging error, enabling more rapid, accurate MR thermometry time courses and arbitrary water flow would constitute a major improvement in the reliability and safety of T-FUS procedures.

This study aims to develop acoustically compatible superparamagnetic nanoparticles to render the coupling water bath invisible to the MR scanner. Previous work has shown that particles of encapsulated magnetite efficiently accelerate the MR transverse relaxation process [X4, X5] (characterized by the R₂ relaxation rate). Several labs have demonstrated a high degree of control over the size, surface properties, and relaxivity of these particles [X6, X7]. When used as an acoustic coupling medium during a T-FUS procedure, these suspensions will rapidly decay the bath's MR signal, rendering it too small relative to the patient's MR signal to produce observable data errors. In addition, by designing 10-100 nm particles with hydrophilic surface properties, these suspensions can negligibly affect both acoustic transmission through the water bath and the generation of unwanted bubble activity (cavitation).

This coupling media can improve the safety and precision of MR guided T-FUS procedures by improving the fidelity of MR image data. The coupling media developed in this work can be put to use by researchers, physicians, and T-FUS device manufacturers, providing a significant improvement to both ET treatments and upcoming T-FUS applications.

### EXPERIMENTAL

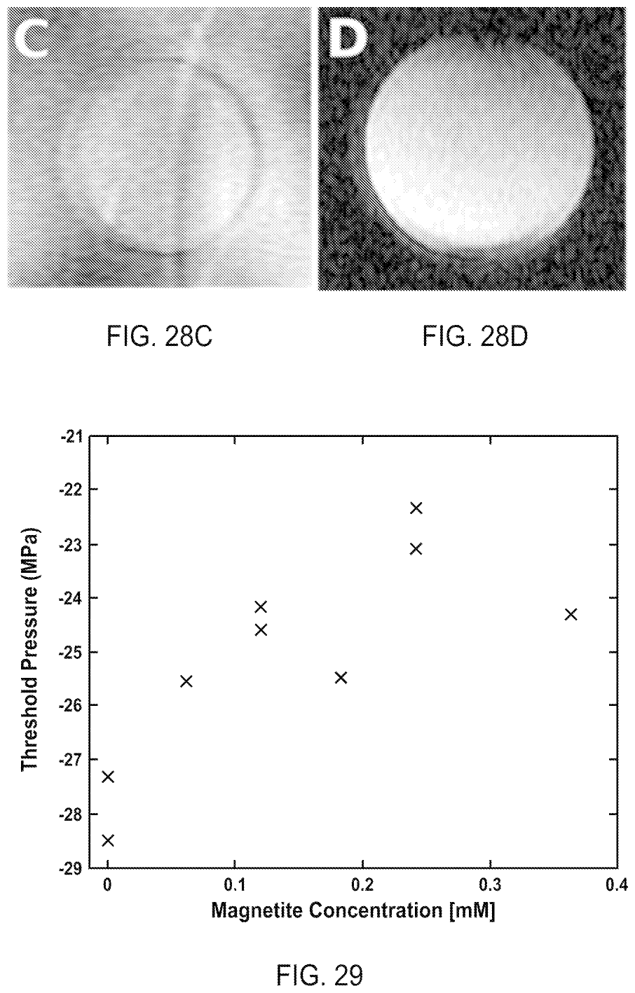

Small-diameter, surface-modified magnetite nanoparticles that both suppress the water bath's MR signal and also disrupt acoustic cavitation upon exposure to ultrasound are developed. Previous work with non-surface-modified, aqueous magnetite agglomerates has shown that magnetite particles can effectively remove water-induced imaging errors, permitting both arbitrary water flow (FIG. 28A-FIG. 28B) and a reduced imaging field-of-view (FIG. 28C-FIG. 28D). However, it was also found that these particles lower the threshold pressure required for spontaneous acoustic cavitation to levels experienced during T-FUS [X8, X9] (FIG. 29). If used during a clinical TFUS procedure, particle-induced cavitation events would potentially impede acoustic transmission, cause skin lacerations, and even falsely signal dangerous cavitation activity inside the patient's vasculature. This study seeks a particle formulation that preserves the high relaxivity of non-modified magnetite while also restoring the cavitation threshold pressure to that of water.

Pure magnetite nanoparticles can seed cavitation events by stabilizing gas bubbles on the surface of the particle agglomerates [X10], thereby reducing the threshold for nucleating cavitation. Therefore, the magnetite particles with small diameters and hydrophilic surface coatings are synthesized that can prevent gas bubble stabilization and increase the cavitation threshold relative to uncoated magnetite particles. This agrees with the work of Smith et al. [X8], who showed that coating pure magnetite particles with a hydrophilic silicon dioxide shell greatly reduced cavitation emissions upon exposure to ultrasound.

Aim 1—Synthesize magnetite nanoparticles to suppress the water bath's MR signal: Magnetite nanoparticles with hydrophilic surface chemistries were synthesized using the co-precipitation method by Mendenhall et al. in which solutions of iron(II) sulfate and iron(III) chloride were reduced using NH₄OH and then sonicated with an aqueous solution of commercially available poly(methacrylic acid) (PMAA) which colloidally disperses the particles [X11]. This produced polymer-stabilized nanoparticles with diameters, as measured by dynamic light scattering, ranging from ˜20-40 nm at pH 6-7. The PMAA adsorbed onto the magnetite and stabilized the nanoparticles via both electrostatic and steric repulsion. Commercially available PMAA with weight-average molecular weight ˜10,000 g/mole is used in this work, comparable to that used previously. This approach is attractive since it can be easily scaled to make large quantities of particles. Aqueous suspensions of the particles can be characterized by thermal gravimetric analysis, dynamic light scattering, transmission electron microscopy, zeta potential measurements, and can undergo standard MR relaxivity measurements [X12,X13]. Using these results, aqueous coupling media with R2 rates of 0.3, 10, 100, and 200 s⁻¹ can be created for further study.

Aim 2—Assess acoustic cavitation threshold pressures. Using a custom fluid chamber, the dependence of the cavitation threshold pressures of the coupling media produced in Aim 1 when exposed to acoustic pulsing regimes commonly used in T-FUS mechanical erosion thermal ablation procedures can be characterized. First, the chamber is placed at the focus of a custom-built, 32 element, 500 kHz, histotripsy transducer and 100 single-cycle histotripsy pulses applied at a repetition rate of 0.5 Hz and at pressure levels ranging from −8 to −42 MPa in 1 MPa increments are deposited. Second, the chamber is placed at the focus of a hemispherical 650 kHz transducer (ExAblate Neuro, InSightec, Haifa Israel) and 100, 10 s “continuous wave” sonications at peak negative pressure levels ranging from 0 to −6 MPa in increments of 0.25 MPa are deposited. Cavitation activity can be monitored using standard methods and the 0.5 cavitation probability threshold pressures can be computed using methods described previously [X14—X17].

If the cavitation thresholds measured in Aim 2 demonstrate a dependence on particle concentration, the concentration with the largest relaxation rate that exhibits a <10% chance of cavitation for a 10 s sonication at 3 MPa peak negative pressure can be selected for use in Aim 3.

Aim 3—In Vitro Validation of Improved T-FUS Guidance Imaging: Next, the hypotheses that the coupling media can allow simultaneous water circulation and MR imaging is tested. Using an MR-compatible, hemispherical, 650 kHz array transducer system (ExAblate Neuro, InSightec, Haifa Israel), common T-FUS MR thermometry and anatomical scans of a gel target can be acquired using a both reduced and full-size imaging fields-of-view under static water flow conditions, continuous flow conditions, and also as a function of time (1-500 s) after the cessation of circulation for each particle concentration produced in Aim 1. The root mean square error (RMSE) and structural similarity index (SSI) [X18] of the resulting images as compared to control images acquired 20 minutes after the cessation of circulation can then be computed.

Conclusion

Aqueous, colloidal particles can be created with a relaxivity of ˜100 s⁻¹ mM⁻¹ that can cause minimal decreases in cavitation threshold pressures for all concentrations and sonication schemes tested. When used as a coupling bath, these particles can greatly improve MRI data accuracy under arbitrary water flow and reduced field-of-view conditions, thereby improving the safety and precision of T-FUS.

### REFERENCES

X1. Hectors S J C G, Jacobs I, Moonen C T W, Strijkers G J, Nicolay K. MRI methods for the evaluation of high intensity focused ultrasound tumor treatment: Current status and future needs. Magn Reson Med. 2016; 75(1):302-317. doi:10.1002/mrm.25758.
X2. Hectors S J C G C G, Jacobs I, Moonen C T W W, Strijkers G J, Nicolay K. MRI methods for the evaluation of high intensity focused ultrasound tumor treatment: Current status and future needs. Magn Reson Med. 2015; 317(1):n/a-n/a. doi:10.1002/mrm.25758.
X3. Ghanouni P, Pauly K B, Elias W J, et al. Transcranial MRI-guided focused ultrasound: A review of the technologic and neurologic applications. Am J Roentgenol. 2015; 205(1):150-159. doi:10.2214/AJR.14.13632.
X4. Gillis P, Koenig SH. Transverse relaxation of solvent protons induced by magnetized spheres: application to ferritin, erythrocytes, and magnetite. Magn Reson Med. 1987; 5(4):323-345. doi:10.1002/mrm.1910050404.
X5. Lee N, Hyeon T. Designed synthesis of uniformly sized iron oxide nanoparticles for efficient magnetic resonance imaging contrast agents. Chem Soc Rev. 2012; 41(7):2575-2589. doi:10.1039/c1cs15248c.
X6. Miles W C, Goff J D, Huffstetler P P, Mefford O T, Riffle J S, Davis R M. The design of well-defined PDMS-Magnetite complexes. Polymer (Guildf). 2010. doi:10.1016/j.polymer.2009.11.022.
X7. Zhang R, Fellows B, Pothayee N, et al. Ammonium Bisphosphonate Polymeric Magnetic Nanocomplexes for Platinum Anticancer Drug Delivery and Imaging with Potential Hyperthermia and Temperature-Dependent Drug Release. J Nanomater. 2018; 2018:1-14. doi:10.1155/2018/4341580.
X8. Smith M J, Ho V H B, Darton N J, Slater N K H. Effect of Magnetite Nanoparticle Agglomerates on Ultrasound Induced Inertial Cavitation. Ultrasound Med Biol. 2009; 35(6):1010-1014. doi:10.1016/j.ultrasmedbio.2008.12.010.
X9. Ho V H B, Smith M J, Slater N K H. Effect of magnetite nanoparticle agglomerates on the destruction of tumor spheroids using high intensity focused ultrasound. Ultrasound Med Biol. 2011; 37(1):169-175. doi:10.1016/j.ultrasmedbio.2010.09.007.
X10. Crum L A. Acoustic cavitation series: part five rectified diffusion. Ultrasonics. 1984; 22(5):215-223. doi:10.1016/0041-624X(84)90016-7.
X11. Mendenhall G D, Geng Y, Hwang J. Optimization of long-term stability of magnetic fluids from magnetite and synthetic polyelectrolytes. J Colloid Interface Sci. 1996; 184(2):519-526. doi:10.1006/jcis.1996.0647.
X12. Pintaske J, Martirosian P, Graf H, et al. Relaxivity of Gadopentetate Dimeglumine (Magnevist), Gadobutrol (Gadovist), and Gadobenate Dimeglumine (MultiHance) in human blood plasma at 0.2, 1.5, and 3 Tesla. Invest Radiol. 2006; 41(3):213-221. doi:10.1097/01.r1i.0000197668.44926.f7.
X13. Rohrer M, Bauer H, Mintorovitch J, Requardt M, Weinmann H-JJ. Comparison of magnetic properties of MRI contrast media solutions at different magnetic field strengths. Invest Radiol. 2005; 40(11):715-724. doi:10.1097/01.r1i.0000184756.66360.d3.
X14. Maxwell A D, Cain C A, Hall T L, Fowlkes J B, Xu Z. Probability of cavitation for single ultrasound pulses applied to tissues and tissue-mimicking materials. Ultrasound Med Biol. 2013; 39(3):449-465. doi:http://dx.doi.org/10.1016/j.ultrasmedbio.2012.09.004.
X15. Allen S P, Hall T L, Cain C A, Hernandez-Garcia L. Controlling cavitation-based image contrast in focused ultrasound histotripsy surgery. Magn Reson Med. 2015; 73(1):204-213. doi:10.1002/mrm.25115.
X16. Vlaisavljevich E, Lin K-W W, Maxwell A, et al. Effects of ultrasound frequency and tissue stiffness on the histotripsy intrinsic threshold for cavitation. Ultrasound Med Biol. 2015; 41(6):1651-1667. doi:10.1016/j.ultrasmedbio.2015.01.028.
X17. Vlaisavljevich E, Gerhardson T, Hall T, Xu Z. Effects of f-number on the histotripsy intrinsic threshold and cavitation bubble cloud behavior. Phys Med Biol. 2017; 62(4):1269-1290. doi:10.1088/1361-6560/aa54c7.
X18. Wang Z, Bovik a C, Sheikh H R, Simmoncelli E P. Image quality assessment: form error visibility to structural similarity. Image Process IEEE Trans. 2004; 13(4):600-612. doi:10.1109/TIP.2003.819861.

### Example 3—Development of Acoustic Coupling Media to Improve Image-Guidance and Treatment Safety in Transcranial Focused Ultrasound Procedures

Surgeries utilizing transcranial focused ultrasound (T-FUS) promise equivalent or better treatment outcomes compared to invasive procedures with the added benefits of reduced morbidity, lower treatment costs, and faster patient recovery times. Because they are non-invasive, T-FUS procedures rely heavily on magnetic resonance imaging (MRI) to guide and control the therapy process, and the quality of intraoperative MR imaging correlates with the degree of control a surgeon has over treatment outcomes [Z1,Z2]. This project aims to improve the safety and efficacy of T-FUS procedures by mitigating a frequent source of MR imaging error: the acoustic coupling water bath.

All T-FUS procedures utilize a water bath to facilitate acoustic transmission from the ultrasound applicator into the patient. However, the coupling bath can also hinder T-FUS procedures by degrading the quality of MR guidance imaging. Specifically, the water bath (1) imposes a large imaging field of view; (2) skews crucial MR calibration parameters such as the transmit RF field amplification factor, magnetic field shimming parameters, and receiver amplification parameters [Z3]; and (3) introduces imaging errors due to inadvertent water flow. The water bath also produces a secondary hindrance by seeding cavitation nuclei. Acoustic emissions from these nuclei falsely signal dangerous cavitation in the patient's vasculature. Upon detection, the surgeon may prematurely attenuate or terminate a given T-FUS sonication.

In this study, a method to simultaneously suppress the coupling bath's ability to both degrade intraoperative MR imaging and seed spurious cavitation is investigated. Nanoparticles can be designed to both rapidly accelerate water's MR signal decay process such that the bath is undetectable during MR imaging and deter the stabilization of gas bubbles on the nanoparticle's surface. These nanoparticles can improve the quality and reliability of MR guidance imaging during T-FUS procedures. These nanoparticles can further satisfy the Food and Drug Administration's (FDA) skin-contact biocompatibility requirements.

Specific Aims

A biocompatible coupling substance with chemical properties that both accelerate the decay of this substance's MR signal and reduce the chance of cavitation upon exposure to a T-FUS procedure is developed.

Aim 1—Synthesize nanoparticles to suppress the water bath's MR signal. Colloidal, magnetite nanoparticles enveloped in a hydrophilic polymeric coating are developed. The magnetite can accelerate the media's transverse relaxation rate to ˜50-100 s⁻¹, rendering it undetectable for most thermometry and anatomical scans. The hydrophilic coating can prevent the particles from aggregating and hosting a stabilized gas bubble. The cavitation thresholds for these particles and their MR relaxivities can be characterized. The impact of these nanoparticles on several relevant measures of MR image quality during T-FUS sonication of gel targets can also be measured.

Aim 2—(A) Perform in vivo validation and (B) establish biocompatibility and safety. (A) Human volunteers can be used to simulate clinical T-FUS thalamotomy procedures while also using the nanoparticles developed in Aim 1. During these procedures, standard MR guidance imaging can be performed. A blinded review panel can then evaluate the impact of the suppressed water bath on clinically relevant image features. The results can be evaluated using both parametric and non-parametric statistical tests. (B) In an effort to facilitate clinical translation and regulatory approval, the toxokinetics and biocompatibility of the nanoparticles can be measured using a preclinical rat model. After their simultaneous exposure to both the nanoparticles developed in Aim 1 and T-FUS sonication, the concentration of iron that has permeated the skin barrier and entered the liver, kidneys, and brain can be estimated. A survival study can also be conducted and any adverse effects on the rats' skin or behavior can be observed.

Background and Significance

Transcranial Focused Ultrasound (T-FUS) is a platform technology that promises noninvasive therapies including neuromodulation, drug transmission across the blood brain barrier, and thermal and mechanical lesioning for a host of clinical indications [Z4-Z6]. The technology's main advantages over current practice include its completely non-invasive nature and its spatial selectivity [Z5]. Currently, T-FUS thalamotomy for essential tremor has received regulatory approval in both the United States and Europe. Meanwhile, treatments for Parkinson's disease and neuropathic pain have received approval in Europe. Procedures for Alzheimer's disease, depression, and epilepsy are currently undergoing clinical trials. If these trials succeed, T-FUS could dramatically improve the morbidity and cost of a large number of transcranial surgical procedures.

Because T-FUS is completely non-invasive, surgeons rely on real-time image guidance to ensure that the treatment process remains localized at the correct location. This task frequently falls to magnetic resonance imaging (MRI), which can provide anatomical imaging, thermometry, tissue characterization, functional assessments, and lesion identification and classification [Z7]. However, if the quality and accuracy of MR imaging decreases, the surgeon's degree of control over treatment safety and efficacy also diminishes [Z1,Z2].

The coupling water bath present during T-FUS procedures remains consistent source of imaging error during intraoperative scanning. For example, the water bath constrains a large imaging field of view, which subsequently limits the rate at which a thermometry or functional MRI time course can be reliably sampled. It also introduces error into the pre-scan calibration parameters such as the magnetic field shimming currents, the radiofrequency transmitter amplifier coefficient, and the receiver amplifier coefficients. Poor calibration leads to blurred or shifted images, degraded signal levels and tissue contrasts, and other image errors [Z3]. Finally, whether by inadvertent patient motion or residual momentum after degassing and circulation, the water bath is almost always in motion. Uncompensated or uncontrolled water flow introduces error into thermometry estimates, blurs tissue contrast, and disrupts the delicate signal correlations used for diffusion tractography and functional MR imaging.

This project aims to use paramagnetic nanoparticles to improve MR imaging guidance and control during T-FUS procedures by eliminating the contribution of the water bath to intraoperative MR image features. Previous works in the literature have shown that particles of encapsulated magnetite efficiently accelerate the MR transverse relaxation process [Z8] (characterized by both the R2 relaxation rate and its corresponding inverse: T₂=R₂⁻¹). Several labs have demonstrated a high degree of control over the size, surface properties, biocompatibility, and relaxivity of these particles [Z9,Z10]. It is estimated that milli-molar concentrations of these particles can be sufficient to accelerate water's R₂ constant from its native 0.3 s⁻¹ to 100 s⁻¹. When added into the water bath, these particles can remove the deleterious effects of the water' signal on a broad class of MRI acquisition schemes, allowing for more accurate pre-scan calibrations, protection against water-motion artifacts, and more time-efficient MR scanning due to a smaller imaging field of view.

The nano-particles present several synergies that can benefit a broad scope of T-FUS applications. For example, the dilute concentration of particles can have a minimal impact on the acoustic and physical properties of the water bath, preserving its coupling and cooling functions while also limiting the chance of a spurious cavitation. The particles can permit simultaneous water circulation and MR thermometry, allowing superior cooling and degassing. Further, a suppressed water bath signal can present fewer confounding image features during MR-CT skull registration. Further, a suppressed water bath can remove a subtle source of 3D MR thermometry error by shortening—by an order of magnitude—the time required for water's MR-signal to reach a steady-state.

This study is designed for rapid clinical translation and use by researchers, physicians, and T-FUS device manufacturers. For example, the particles can be designed to both suppress the water bath and remain resistant to cavitation across the broad range of sonication parameters used by disparate T-FUS applications (e.g., neuromodulation, blood brain barrier opening, histotripsy, and thermal ablation). Further, a safety and biocompatibility study that is designed to facilitate future clinical translation and regulatory approval can be conducted.

Results

Efficacy of Magnetite Nanoparticles

Uncoated magnetite nanoparticle aggregates during T-FUS procedures in gel targets were previously investigated. Using a continuously circulated 0.25 mM nanoparticle concentration in the water bath, these magnetite particles were able to suppress more than 90% of the water bath signal for T2-weighted spin echo anatomical scans and T2* weighted thermometry scans (FIG. 10-FIG. 13).

Additionally, with the particles in the water bath, MR thermometry was performed simultaneous to 10 s long, continuous wave sonications of a gel target over a broad range of transmitted acoustic powers. The presence of the nanoparticles attenuated the temperatures of the gel target (averaged over 3 pixels) by 2° C. for all transmitted acoustic powers above 200 W relative to sonications with a standard degassed water bath (FIG. 19). These results indicate that small dilutions of magnetite nanoparticles do not greatly inhibit the propagation of ultrasound through the water bath.

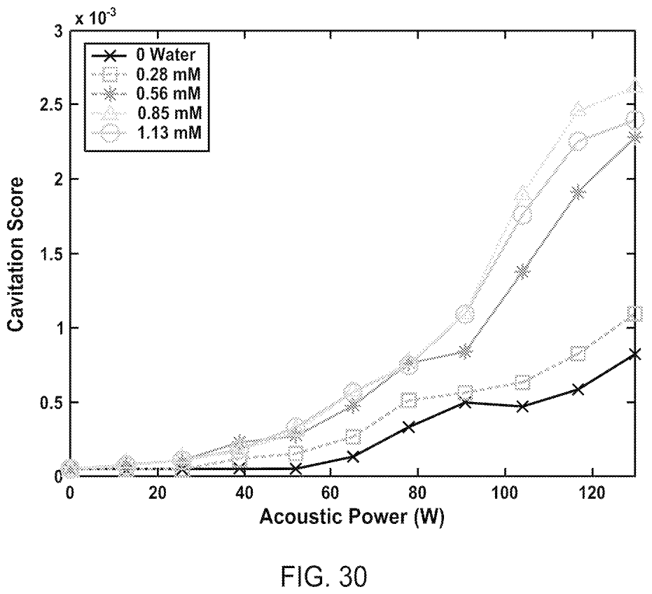

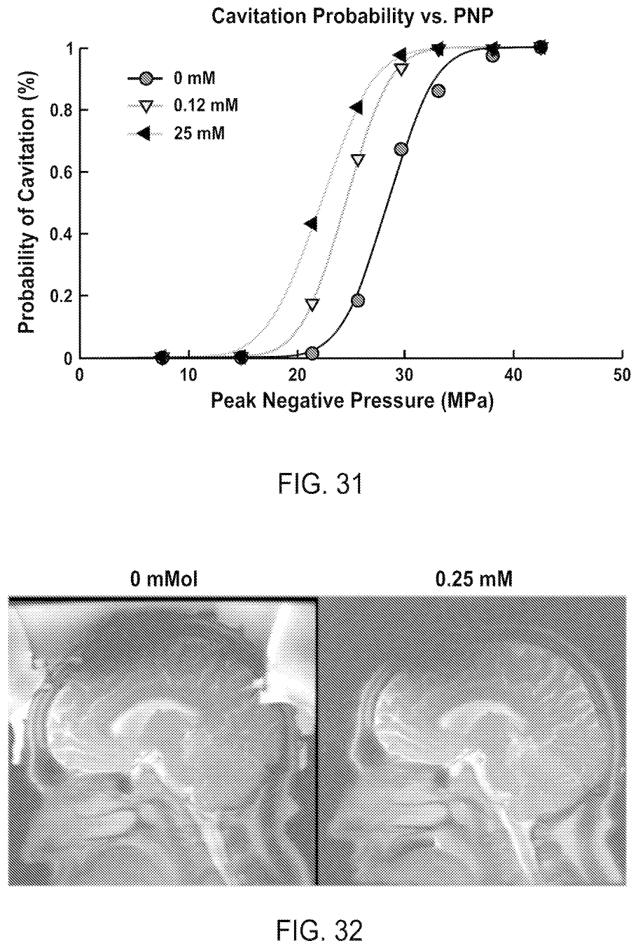

However, magnetite particles have been previously shown to seed cavitation nuclei at even very low amplitude sonications [Z11]. Further, over the course of the previous experiments, enhanced acoustic emissions were observed in the water bath. It was subsequently shown that sonicating aqueous magnetite particles can produce both stable and inertial cavitation emissions across a broad range of particle concentrations and transmitted acoustic powers. (FIG. 30). It was also found that the presence of pure magnetite particles can lower the intrinsic inertial cavitation pressure threshold (FIG. 31). An ideal nanoparticle design will both suppress the water bath signal and remain resistant to cavitation.

It is hypothesized that pure magnetite nanoparticles can seed cavitation nuclei by (1) stabilizing gas bubbles on the surface of the particle aggregates [Z12] and (2) generating weak points in the hydrogen bond network among water molecules surrounding the aggregates [Z13]. Magnetite particles with hydrophilic surface coatings can disrupt these two mechanisms, increasing the cavitation threshold relative to uncoated magnetite. In the literature, work by Smith et al. [Z11] showed that coating pure magnetite particles with a hydrophilic silicone dioxide shell greatly reduced cavitation emissions upon exposure to low-amplitude, continuous wave ultrasound, likely by reducing the occurrence of stabilized gas bubbles. Building off of these results, colloidal magnetite nanoparticles with hydrophilic shells that can both accelerate the R₂ relaxation rate of water and prevent the formation of cavitation nuclei can be constructed.

In Vivo Validation

Magnetite nanoparticles were previously used to suppress the water bath while scanning human volunteers. Upon scanning, fewer flow artifacts and a reduced field of view were observed (FIG. 32). The quality of the images acquired during a nominal T-FUS procedure as a function of water bath composition can be further evaluated.

Biocompatibility and Safety

The literature contains extensive documentation on the safety and biocompatibility of magnetite nanoparticle variants in the context of injectable contrast agents [Z14—Z16], detailing their distribution, kinetics, and toxicity [Z17]. Possible vectors for patient harm include oxidative DNA and cellular damage from nanoparticle breakdown products, local iron overload, and cytotoxicity [Z14, Z15]. However, clinical trials of dextran coated magnetite nanoparticles demonstrate high patient tolerability with very few instances of the above mentioned side effects or adverse events [Z17]. In vitro studies of cellular exposure to aqueous magnetite solutions indicate that a variety of cells types express cytotoxic responses including losses in cell viability, membrane integrity, and increased inflammatory cytokine expression. These results varied with both the concentration and surface coating of the particles [Z14, Z15, Z18, Z19].

In contrast, the existing body of literature addressing the potential toxicity of dermal exposure to magnetite nanoparticles is less satisfactory [Z20]. However, the existing studies indicate that intact skin may be a fairly effective barrier against nanoparticle infiltration for particles between 5 and 20 nm in diameter [Z21,Z22]. In one study, aqueous solutions of hydrophilic magnetite nanoparticles with diameters of less than 20 nm were allowed to diffuse across ex vivo full thickness human abdominal skin. Electron microscopy indicated that the nanoparticles could penetrate the stratum corneum and hair follicles but only very rarely penetrated to deeper layers such as the stratum granulosum [Z23]. A second study found no penetration beyond the stratum corneum [Z24]. In another study, washing with soap and water removed at least 85% of dried magnetite nanoparticles from the stratum corneum [Z25]. One case report of cutaneous contact with an extremely high concentration of magnetite particles resulted in only temporary skin discoloration [Z26].

It is also possible that the skin barrier may be compromised via a sonophoresis effect or by inadvertent skin wounds as a result of shaving the patient's hair. For example, after making an incision through the stratum corneum of ex vivo skin, uncoated iron oxide particles have been observed to diffuse through the intra and extra cellular spaces of the stratum granulosum [Z27]. Meanwhile, sonophoresis has demonstrated modest success in enhancing the skin penetration and permeation of non-iron metallic nano particles [Z28].

Experimental Design and Methods

Aim 1: Synthesize Nanoparticles to Suppress the MR Water Bath Signal

Magnetite nanoparticles accelerate the MR transverse relaxation process via the diffusion of water through the complex magnetic microenvironment near the particle surface [Z29,Z30]. In general, the relaxivity of a nanoparticle design increases with the size of the particle and the thinness of its encapsulating shell [Z8]. Likewise, the ability of a solid particle to seed stabilized gas bubbles also depends on the size and chemical makeup of the surface of the nanoparticle [Z31]. For example, the cavitation threshold pressure associated with solid particles tends to decrease with increased size of the stabilized gas bubble [Z32] and the hydrophobicity of the particle's shell. An important part of this project is designing a balance between these relationships.

In this aim, small, hydrophobic, colloidal magnetite nanoparticles are designed and their acoustic cavitation and MR imaging properties are characterized under various acoustic pulsing schemes and magnetic field strengths. Their efficacy in improving MR imaging metrics during T-FUS ablations of gel targets can then be demonstrated.

MR Characterization: The transverse and longitudinal relativities of various concentrations of the nanoparticles can be estimated in both 1.5 and 3 T MR scanners using multi-spin echo and inversion recovery sequences. The multi-spin-echo sequence can be repeated 3 times and can use echo spacings of 2.5, 10, and 20 ms to acquire echo times varying from twice the echo spacing to 500 ms. The inversion recovery sequences can employ inversion times ranging from 10 to 4000 ms. Both sequences can have repetition times exceeding 8 seconds. Each concentration's relaxation constants can be estimated using a least-squares fit to a single compartment model [Z33]. The relaxivities of the particles can then be estimated by a linear least-squares fit of the estimated relaxation parameters to the concentration of the nanoparticles.

Cavitation Characterization: The cavitation threshold of water baths containing magnetite nanoparticles can be characterized by sonicating a small chamber filled with the particles using 4 different acoustic pulsing regimes. First, a single-cycle, “intrinsic histotripsy” pulse (˜110/40 MPa peak positive and peak negative pressures) pulsed at 2 Hz using a 500 kHz transducer can be used. Second, a five-cycle, “shock scattering histotripsy” pulsing scheme (˜80/13 MPa peak positive and peak negative pressures) using a 700 kHz transducer can be used. Third, a 10 s “continuous wave” pulsing scheme (10/4 MPa peak positive and peak negative pressures) using a hemispherical 650 kHz transducer (ExAblate Neuro, Insightec, Haifa Israel) can be used. Finally, a 20 s “continuous wave” pulsing scheme using a 250 kHz transducer (Insightec, Haifa Israel) suitable for blood brain barrier disruption (2/2 MPa peak positive and peak negative pressures) can be used. Cavitation detection can be achieved by passive cavitation detection (PCD) emissions and by optical cameras for sonication schemes 1 and 2 and by only PCD for schemes 3 and 4.

For each sonication scheme, the acoustic pressures can be raised from 0 MPa to the maximum peak positive and negative pressures described above. At each pressure level, the experiment can be repeated 100 times. The cavitation threshold can be computed as the lowest sonication pressure level that reports cavitation in 50 or more of the repeated experiments.

MR-Guided T-FUS Validation: The nanoparticles can improve MR guidance by reducing the imaging field of view, reducing water motion artifact, and by improving MR prescan calibrations. These claims can be tested by using an MR-compatible, hemispherical, 650 kHz array transducer system (ExAblate Neuro, Insightec, Haifa Israel) to sonicate gel targets while varying the concentration of nanoparticles suspended in the water bath. Using MR-compatible thermocouples as an experimental control, the accuracy and repeatability of small-field-of-view (15 cm×15 cm) MR thermometry scans can be tested. The variance of both phase and magnitude images of a uniform gel target as a function of time (1-500 s) after the cessation of water bath circulation can be computed. Finally, the uniformity of static and transmit field maps acquired after standard pre-scan calibrations can be measured as a function of water bath composition.

Outcome: Based on preliminary data, nanoparticles with relaxivities of 100 mM s⁻¹ can be created. A 1 mM concentration of these particles can then suppress the water bath at levels equivalent to those shown in FIG. 10-FIG. 13. The coated particles can also express cavitation thresholds close to that of degassed water for sonication schemes 3 and 4. There can be a small drop in cavitation threshold pressure for schemes 1-2 due to inhomogeneities in the hydrogen bond structure on the particle's wetted surface. The suppressed water bath can enable small field-of-view thermometry, reduce water motion artifacts, and improve the uniformity of the static and transmit fields produced after a prescan calibration.

Potential Problems and Alternative approach: If it is determined that having the particles touch the transducer surface is detrimental, plastic barriers can be used between the water bath and the transducer surface for all MR imaging experiments. Further, for the sonication of gel targets, the water bath can be separated into two hemispherical shells using a plastic barrier; the section in contact with the transducer can contain degassed water and the section in contact with the gel target can contain the nanoparticles.

Aim 2: In Vivo validation and Safety

This aim proposes to explicitly test the hypothesis that including nanoparticles in the coupling water bath can improve the quality and reliability of MR guidance imaging in a clinical T-FUS setting. This is in contrast to the experiments proposed in Aim 1, which can measure the impact the nanoparticles have on general image quality metrics. A secondary goal of this aim is to demonstrate the safety and biocompatibility of these nanoparticles. Basic toxicology and biocompatibility tests can be performed to estimate any risks these particles may pose during a clinical T-FUS procedure. The results produced by these studies can facilitate future clinical translation and regulatory approval.

Clinical T-FUS Validation: Four human volunteers can be used to simulate clinical T-FUS thalamotomies and measure the impact of the water bath design in clinically relevant MR guidance imaging features. Prior to the simulated procedure, volunteers undergo a ultra-short-echo-time (UTE) MRI to produce a pseudo-computed tomography (CT) model of their skulls [Z34]. The use of UTE MRI can reduce the volunteer's exposure to ionizing radiation while simultaneously producing a skull estimate with almost the same morphology as that produced by a CT scan [Z35].

Each volunteer is then coupled to an MR compatible T-FUS device (Insightec Exablate Neuro, Haifa, Israel) according to standard clinical procedures. The water bath either includes (n=2) or excludes (n=2) the nanoparticles. The procedure is then carried out normally with the exception that the acoustic amplifiers will be disabled, preventing the transmission of ultrasound into the volunteer. More specifically, the pseudo-CT skull model can be registered to intraoperative imaging; clinical anatomy scans can be used to plan lesioning targets off of anterior commissure and posterior commissure lines; and clinical MR thermometry sequences can be used to estimate the temperature at the focal spot. Finally, T2-weighted MR imaging can be used to estimate the formation of edema at the sonication target and T2*-weighted images can be used to identify potential hemorrhages. All scanning parameters, except for pre-scan calibrations, will be held invariant with water bath composition.

All imaging sets can then be brought before a blinded panel of three experienced T-FUS clinicians. To prevent bias, the water bath, if visible, can be cropped from each image prior to review. The panel can evaluate each image on a 5 point scale. Differences in image quality ratings can be assessed using a Kruskal-Wallis test. Further, the panel can perform pairwise comparisons of images acquired with and without the nanoparticles in the water bath. These comparisons can be assessed using a Wilcoxson signed rank test with Bonferroni correction for multiple comparisons.

Biocompatibility and Safety: Anticipating FDA safety requirements, the toxokinetics and biocompatibility of the nanoparticles can be measured using a preclinical rat model. A total of 40 rats have their scalps shaved and are then exposed to different water bath compositions for 3 hours (30 to a water bath with nanoparticles designed to accelerate R2 to 100 s⁻¹, 10 to a water bath without any nanoparticles). An additional 5 rats are not exposed to the water bath as a control. After exposure one third of the rats are immediately sacrificed. The portions of the skin exposed to the water bath, the kidney, the liver, and the brains of these animals are preserved, sectioned, and processed using a Prussian blue staining to detect iron deposits. Transmission electron microscopy and optical microscopy can then be used to estimate the relative proportions of iron within these organs [Z23,Z36]. The remaining animals survive for 28 days. Their skin and behavior is assessed for any abnormalities. Finally, these animals and the 10 unexposed controls are sacrificed and subjected to the same analysis as the acute cohort. Differences in the relative concentration of iron deposits are assessed using two-tailed t-tests.

Outcomes: The nanoparticles can produce higher scores on anatomical imaging, CT-MR registration, and hemorrhage detection scans compared to images acquired without the water bath. However, the thermometry scans, which are already fairly robust, can demonstrate image quality independent of the water bath composition. In the biosafety experiments, the particles can penetrate the stratum corneum of the skin but not the liver, kidneys, or brain. Further, the survived animals can present skin discoloration and no other adverse effects.

Summary of Measurable Results

1. The relaxivity of the designed nanoparticles.

2. The cavitation thresholds of the nanoparticles for each sonication scheme.

3. The temperatures achieved in sonications of gel targets while using various water bath designs acquired using thermocouples and MR thermometry.

4. The signal variance of images of a gel target as a function of time between image acquisition and the cessation of water bath circulation

5. The variance of the measured static and transmit magnetic fields in a gel target as a function of water bath design

6. Review panel assessments of the quality of images acquired for MR-CT registration, target localization, thermometry, and edema and hemorrhage detection as a function of water bath composition

7. Relative concentrations of iron deposits in the skin, liver, kidney, and brain of rats exposed to various water bath compositions.

8. Abnormalities in behavior and skin appearance of rats at 1 to 28 days after exposure to water baths of different compositions.

Impact

This project aims to improve all MR-guided FUS applications. The technical innovations described herein can minimize deleterious effects of the acoustic coupling bath on intraoperative MR guidance imaging during T-FUS. They can also minimize the risk of spurious cavitation events. Finally, these innovations can allow for higher quality guidance images, faster thermometry time courses, and simultaneous water bath circulation and MR imaging. Altogether, this study can facilitate the rapid translation of a design that can improve the safety of transcranial MR-guided FUS procedures.

### REFERENCES

Z1. Hectors S J C G C G, Jacobs I, Moonen C T W W, Strijkers G J, Nicolay K. MRI methods for the evaluation of high intensity focused ultrasound tumor treatment: Current status and future needs. Magn Reson Med. 2015; 317(1):n/a-n/a. doi:10.1002/mrm.25758.
Z2. Ghanouni P, Pauly K B, Elias W J, et al. Transcranial MRI-guided focused ultrasound: A review of the technologic and neurologic applications. Am J Roentgenol. 2015; 205(1):150-159. doi:10.2214/AJR.14.13632.
Z3. Odéen, Henrik; Patil, Sunil; Bolster, Bradley; Bhat Himanshu; Parker D L. Evaluation and tradeoffs of 2D and 3D Cartesian MR temperature imaging (MRTI) for brain applications. In: 6th International Symposium on Focused Ultrasound. Reston, Va.; 2018:BR-46.
Z4. Ghanouni P, Pauly K B, Elias W J, et al. Transcranial MRI-Guided Focused Ultrasound: A Review of the Technologic and Neurologic Applications. Am J Roentgenol. 2015; 205(1):150-159. doi:10.2214/AJR.14.13632.
Z5. Ellis S, Rieke V, Kohi M, Westphalen A C. Clinical applications for magnetic resonance guided high intensity focused ultrasound (MRgHIFU): Present and future. J Med Imaging Radiat Oncol. 2013; 57(4):391-399. doi:10.1111/1754-9485.12085.
Z6. Colen R R, Sahnoune I, Weinberg J S. Neurosurgical Applications of High-Intensity Focused Ultrasound with Magnetic Resonance Thermometry. Neurosurg Clin N Am. 2017; 28(4):559-567. doi:10.1016/j.nec.2017.05.008.
Z7. Hectors S J C G, Jacobs I, Moonen C T W, Strijkers G J, Nicolay K. MRI methods for the evaluation of high intensity focused ultrasound tumor treatment: Current status and future needs. Magn Reson Med. 2016; 75(1):302-317. doi:10.1002/mrm.25758.
Z8. Lee N, Hyeon T. Designed synthesis of uniformly sized iron oxide nanoparticles for efficient magnetic resonance imaging contrast agents. Chem Soc Rev. 2012; 41(7):2575-2589. doi:10.1039/c1cs15248c.
Z9. Balasubramaniam S, Kayandan S, Lin Y N, et al. Toward design of magnetic nanoparticle clusters stabilized by biocompatible diblock copolymers for T2-weighted MRI contrast. Langmuir. 2014; 30(6):1580-1587. doi:10.1021/1a403591z.
Z10. Wu W, Jiang C Z, Roy V A L. Designed synthesis and surface engineering strategies of magnetic iron oxide nanoparticles for biomedical applications. Nanoscale. 2016; 8(47):19421-19474. doi:10.1039/c6nr07542h.
Z11. Smith M J, Ho V H B, Darton N J, Slater N K H. Effect of Magnetite Nanoparticle Agglomerates on Ultrasound Induced Inertial Cavitation. Ultrasound Med Biol. 2009; 35(6):1010-1014. doi:10.1016/j.ultrasmedbio.2008.12.010.
Z12. Xu S, Qiao Y, Liu X, Church CC. Cavitation In Biomedicine: Principles and Techniques. In: Wan M, Feng Y, Ter Haar G, eds. Dordrecht: Springer; 2015:1-46. doi:10.1007/978-94-017-7255-6.
Z13. Li B, Gu Y, Chen M. Cavitation Inception of Water with Solid Nanoparticles: A Molecular Dynamics Study. Ultrason Sonochem. 2018; (January):0-1. doi:10.1016/j.ultsonch.2018.10.036.
Z14. Liu G, Gao J, Ai H, Chen X. Applications and potential toxicity of magnetic iron oxide nanoparticles. Small. 2013; 9(9-10):1533-1545. doi:10.1002/sm11.201201531.
Z15. Singh N, Jenkins G J S, Asadi R, Doak S H. Potential toxicity of superparamagnetic iron oxide nanoparticles (SPION). Nano Rev. 2010; 1(1):5358. doi:10.3402/nano.v1i0.5358.
Z16. Bernd H, De Kerviler E, Gaillard S, Bonnemain B. Safety and Tolerability of Ultrasmall Superparamagnetic Iron Oxide Contrast Agent. Invest Radiol. 2009; 44(6):336-342. doi:10.1097/RLI.0b013e3181a0068b.
Z17. Arami H, Khandhar A, Liggitt D, Krishnan K M. In vivo delivery, pharmacokinetics, biodistribution and toxicity of iron oxide nanoparticles. Chem Soc Rev. 2015; 44(23):8576-8607. doi:10.1039/c5cs00541h.
Z18. Murray A R, Kisin E, Inman A, et al. Oxidative Stress and Dermal Toxicity of Iron Oxide Nanoparticles In Vitro. Cell Biochem Biophys. 2013; 67(2):461-476. doi:10.1007/s12013-012-9367-9.
Z19. Hildebrand H, Kühnel D, Potthoff A, Mackenzie K, Springer A, Schirmer K. Evaluating the cytotoxicity of palladium/magnetite nano-catalysts intended for wastewater treatment. Environ Pollut. 2010; 158(1):65-73. doi:10.1016/j.envpol.2009.08.021.
Z20. Korinth G, Drexler H. Penetration of Nanoparticles through Intact and Compromised Skin. Nanomaterials. 2013:37-42. doi:10.1002/9783527673919.ch4.
Z21. Larese Filon F, Bello D, Cherrie J W, Sleeuwenhoek A, Spaan S, Brouwer D H. Occupational dermal exposure to nanoparticles and nano-enabled products: Part I-Factors affecting skin absorption. Int J Hyg Environ Health. 2016; 219(6):536-544. doi:10.1016/j.ijheh.2016.05.009.
Z22. Wang M. Evaluation of immunoresponses and cytotoxicity from skin exposure to metallic nanoparticles. 2018:4445-4459.
Z23. Baroli B, Ennas M G, Loffredo F, Isola M, Pinna R, Lopez-Quintela M A. Penetration of metallic nanoparticles in human full-thickness skin. J Invest Dermatol. 2007; 127(7):1701-1712. doi:10.1038/sj.jid.5700733.
Z24. Choi K-J, Park B, Lee S, Wang Q, Lee S. Morphological Studies of Penetration Pathways via Stratum corneum and Hair Follicles using Nano-sized Iron Oxide. Microsc Microanal. 2017; 23(S1):1374-1375. doi:10.1017/5143192761700753X.
Z25. Lewinski N A, Berthet A, Maurizi L, Eisenbeis A, Hopf N B. Effectiveness of hand washing on the removal of iron oxide nanoparticles from human skin ex vivo. J Occup Environ Hyg. 2017. doi:10.1080/15459624.2017.1296238.
Z26. Cohen P R, Arfa K S. Ferrofluid-associated Cutaneous Dyschromia: Discoloration of Hand and Fingers Following Accidental Exposure to Ferromagnetic Fluid. J Clin Aesthet Dermatol. 2016; 9(3):51-54.
Z27. Lee S E, Choi K J, Menon G K, et al. Penetration pathways induced by low-frequency sonophoresis with physical and chemical enhancers: Iron oxide nanoparticles versus lanthanum nitrates. J Invest Dermatol. 2010; 130(4):1063-1072. doi:10.1038/jid.2009.361.
Z28. Labouta H I, Schneider M. Interaction of inorganic nanoparticles with the skin barrier: current status and critical review. Nanomedicine. 2013; 9(1):39-54. doi:10.1016/j.nano.2012.04.004.
Z29. Brooks R a, Moiny F, Gillis P. On T2-shortening by weakly magnetized particles: the chemical exchange model. Magn Reson Med. 2001; 45(6):1014-1020.
Z30. Brooks R A, Moiny F, Gillis P. On T2-shortening by strongly magnetized spheres: A partial refocusing model. Magn Reson Med. 2002; 47(2):257-263. doi:10.1002/mrm.10059.
Z31. Crum L A. Acoustic cavitation series: part five rectified diffusion. Ultrasonics. 1984; 22(5):215-223. doi:10.1016/0041-624X(84)90016-7.
Z32. Kwan J J, Graham S, Myers R, Carlisle R, Stride E, Coussios CC. Ultrasound-induced inertial cavitation from gas-stabilizing nanoparticles. Phys Rev E—Stat Nonlinear, Soft Matter Phys. 2015; 92(2):1-5. doi:10.1103/PhysRevE.92.023019.
Z33. Barral J K, Gudmundson E, Stikov N, Etezadi-Amoli M, Stoica P, Nishimura D G. A robust methodology for in vivo T1 mapping. Magn Reson Med. 2010; 64(4):1057-1067. doi:10.1002/mrm.22497.
Z34. Johansson A, Karlsson M, Nyholm T. CT substitute derived from MRI sequences with ultrashort echo time. Med Phys. 2011; 38(5):2708-2714. doi:10.1118/1.3578928.
Z35. Miller G W, Eames M, Snell J, Aubry J-F. Ultrashort echo-time MRI versus CT for skull aberration correction in MR-guided transcranial focused ultrasound: In vitro comparison on human calvaria. Med Phys. 2015; 42(5):2223-2233. doi:10.1118/1.4916656.
Z36. Allen S P, Vlaisavljevich E, Shi J, et al. The response of MRI contrast parameters in in vitro tissues and tissue mimicking phantoms to fractionation by histotripsy. Phys Med Biol. 2017; 62(17):7167-7180. doi:10.1088/1361-6560/aa81ed.

### Example 4

The purpose of this project is to develop an acoustic coupling bath that is effectively invisible to MRI scans and also remains acoustically compatible with clinical transcranial FUS procedures. This remains important to the clinical translation of FUS because the commonly used acoustic coupling bath of degassed water persistently degrades the quality of MR guidance imaging. This project proposes an acoustic coupling bath doped with iron oxide nanoparticles that 1) have a high ability to decay MRI signals, (as quantified by the r₂ relaxivity), 2) have diameters less than 100 nm, and 3) are coated with a hydrophilic coating. It was hypothesized that these three features can produce a lightly doped water bath with identical mechanical, acoustic, thermal, biocompatibility, and electromagnetic properties as water, but with virtually no observable appearance or effect on guidance MR imaging. Finally, the low particle concentrations, small particle sizes, and specific coating can prevent the particles from stabilizing gas bubbles and seeding cavitation nuclei in the transducer's pre-focal field.

Overview of Particle Design

Iron oxide particles were synthesized using the coprecipitation method. To produce a hydrophilic surface coating, the particles were mixed with polymethacrylic acid and then placed in a water bath sonicator (Ultrasonic Bath, 15337410, 110 W, 40 kHz, Fisher Scientific) for 30 minutes. To produce small particles and improve purity, the resulting suspension was washed using magnetic sedimentation and deionized water. The suspension was then centrifuged (Sorvall Legend X1R, ThermoFisher Scientific) for 60 minutes at 5000 rcf. The sizes of the resulting particles were then analyzed using dynamic light scattering (Zetasizer Nano ZS).

The particles were found to be very monodisperse, with a mean hydrodynamic diameter of 31±1 nm. The mean diameter of the particles varied by less than 10% after storage in a refrigerator for 90 days, suggesting that the particles remain highly stable and well dispersed throughout the medium.

Relaxivity Estimates

To measure the relaxivity of these particles, 10 mL of 0.9, 0.45, 0.225, and 0.09 mM Fe dilutions were stored in conical Eppendorf tubes. These were then placed in the bore of a 3 T MRI scanner (MR 750, GE, Waukesha, Wis.) and subject to inversion recovery and multi-spin-echo sequences, which are the gold standard methods to estimate the T1 and T2 relaxation times of a given sample.

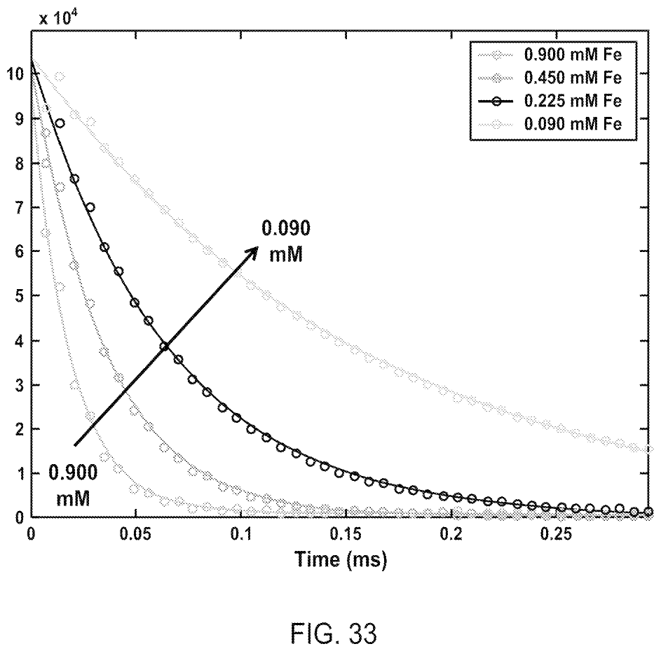

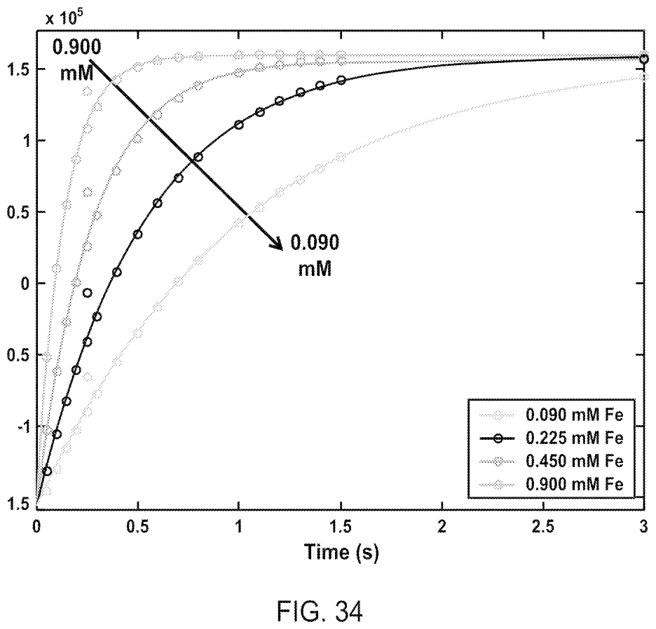

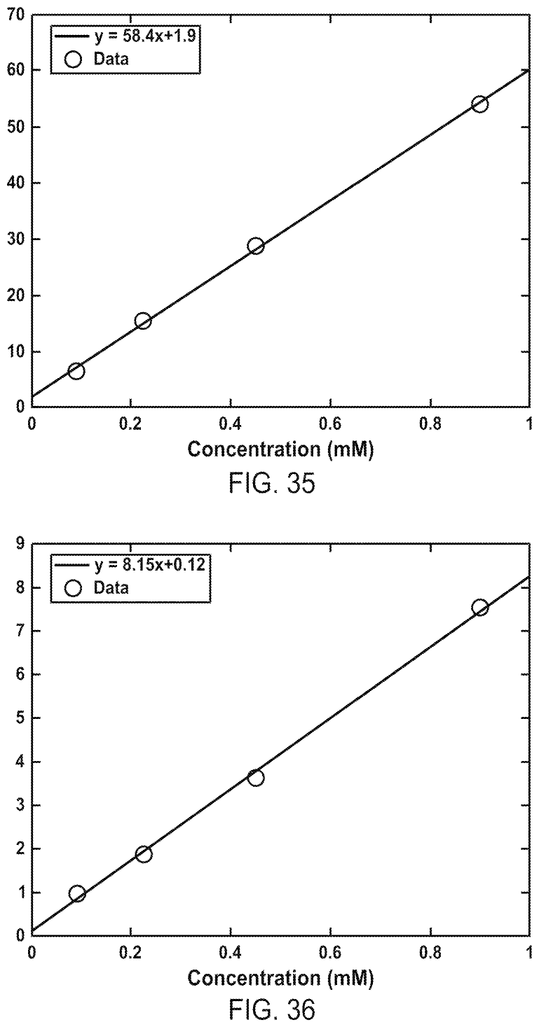

The sequences did a very good job of sampling both exponential decay curves for all but the T2 decay curve of the 0.09 mM samples. All sampled relaxation curves were very well described by mono-exponential decay. Some examples of the sampled data points (circles) and their corresponding least squares fits (lines) are shown in FIG. 33 and FIG. 34. The characteristic decay times from each of these curves can be plotted as a function nanoparticle concentration, as shown in FIG. 35 and FIG. 36, where the slope of the resulting line is the relaxivity of the particles.

The relaxivity of these particles compared to commercial particles obtained during a previous iteration of the project (Example 1) are displayed in Table 1. As can be observed, the particles generated in this example possess a much smaller relaxivity than the commercial particles used in Example 1. This means that the particles used in this example will be less efficient than the commercial particles at suppressing the MR signal of the coupling bath. Therefore higher concentration of the particles tested here will be required to achieve the same effects as those achieved with the commercial iteration.

TABLE 1
The r1 and r2 relaxivity of particles
from Example 1 and Example 2.

| | r₂ Relaxivity | r₁ Relaxivity |
|---|---|---|
| Particle Source | (s⁻¹ mM⁻¹ Fe) | (s⁻¹ mM⁻¹ Fe) |
| Previous Iteration (Example 1) | 175 | Not Measured |
| 240 nm diameter, Commercial Source | | |
| Current Iteration, 31 nm diameter | 58.4 ± 0.03 | 8.19 ± 0.15 |

The relatively smaller relaxivity is likely because the commercial particles would aggregate into clumps with a diameter about 7 times larger than the particles tested here. The larger the particle, or aggregation of particles, the larger the spatial extent of the magnetic field gradient the particle imposes as the magnetic field smoothly varies from the magnetic field strength at its surface (as calculated using the particle's magnetic susceptibility) and the ambient magnetic field strength. The field gradient, while microscopic, is what enables iron oxide to so efficiently accelerate T₂ MRI signal decay. Thus, larger particles tend to exhibit larger relaxivities and smaller particles, such as those discussed in this report, produce relatively smaller relaxivities.

Pulsed Cavitation Threshold Results

Insonating a sample with low duty-cycle, single acoustic pulses remains one of the best methods to understand the cavitation threshold pressure for a given acoustic frequency. The same dilutions tested during the relaxivity experiments described above were also subjected to single-cycle acoustic cavitation tests.

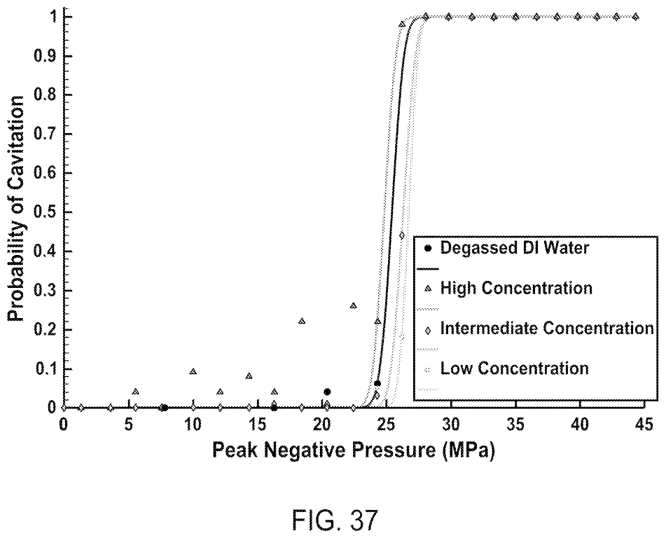

Degassed solutions of 0.9, 0.09, and 0.009 mM Fe concentrations were placed in a chamber at the focused of 500 kHz, intrinsic threshold, histotripsy transducer. Passive acoustic detectors and a camera were used to record cavitation events, which were then tabulated. The relative fraction of acoustic pulses yielding a cavitation event at a particular peak negative pressure was labeled the “probability of cavitation.” The results are displayed in FIG. 37.

These experiments yielded an estimate of the probability of cavitation as a function of peak negative acoustic pressure. The results indicated that the 0.5 cavitation probability threshold pressure remained invariant with nanoparticle concentration (FIG. 37). However, for the 0.9 mM concentration (labeled “High Concentration” in FIG. 37), non-trivial cavitation probabilities occurred at pressures below the 0.5 probability threshold pressure. These results suggest that some care must be taken to ensure that the prefocal acoustic field does not reach these peak negative pressures.

Cavitation threshold data, derived from FIG. 37, are presented Table 2. It appears that the 0.5 cavitation probability threshold pressure, pₜ, is invariant with particle concentration over the concentration ranges tested here. pₘᵢₙ refers to the minimum pressure at which a non-zero cavitation probability was observed. The term σ is a measure of how gradually the threshold curve appears to increase with negative pressure.

TABLE 2
Cavitation threshold data.

| | pₜ | pₘᵢₙ | |
|---|---|---|---|
| Sample | (MPa) | (MPa) | σ |
| Degassed DI Water | 25.4 | 20.4 | 0.72 |

| | | | | |
|---|---|---|---|---|
| 0.9 | mM Fe | 24.8 | 5.5 | 0.69 |
| 0.09 | mM Fe | 26.3 | 16.3 | 0.72 |
| 0.009 | mM Fe | 26.7 | 24.3 | 0.56 |

Continuous Wave Cavitation Threshold Results

The same particles, with the same concentrations, were subjected to continuous wave cavitation experiments. These are a modification of the pulsed experiments described above where the particles in the chamber are insonated for 10 s using a continuous wave acoustic pulse at 650 kHz. Passive acoustic detectors then record the relative fraction of the 10 s insonation where cavitation events are thought to occur. These curves, while not equivalent to a “probability of cavitation,” also demonstrate a sigmoidal shape and can suggest the frequency of cavitation events occurring at a given peak negative pressure.

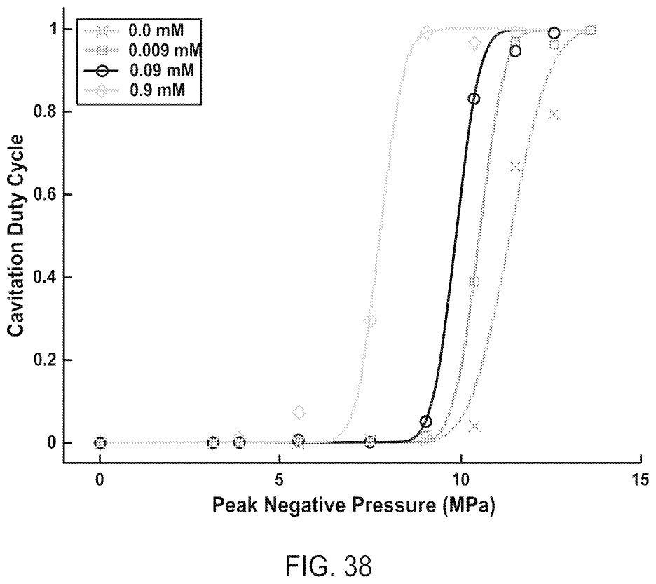

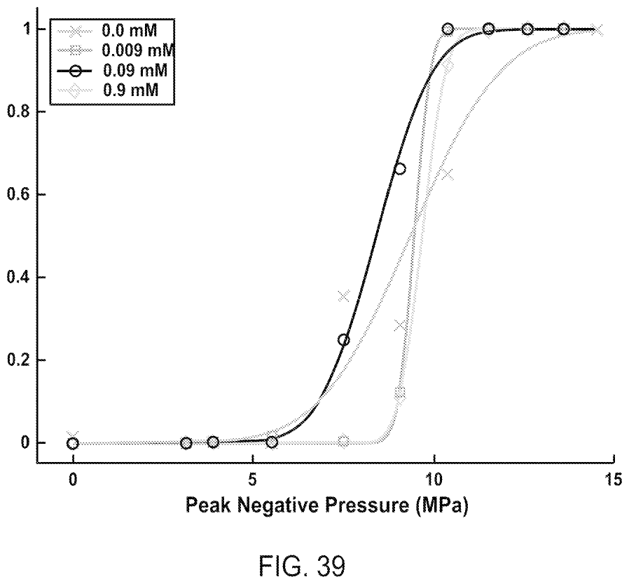

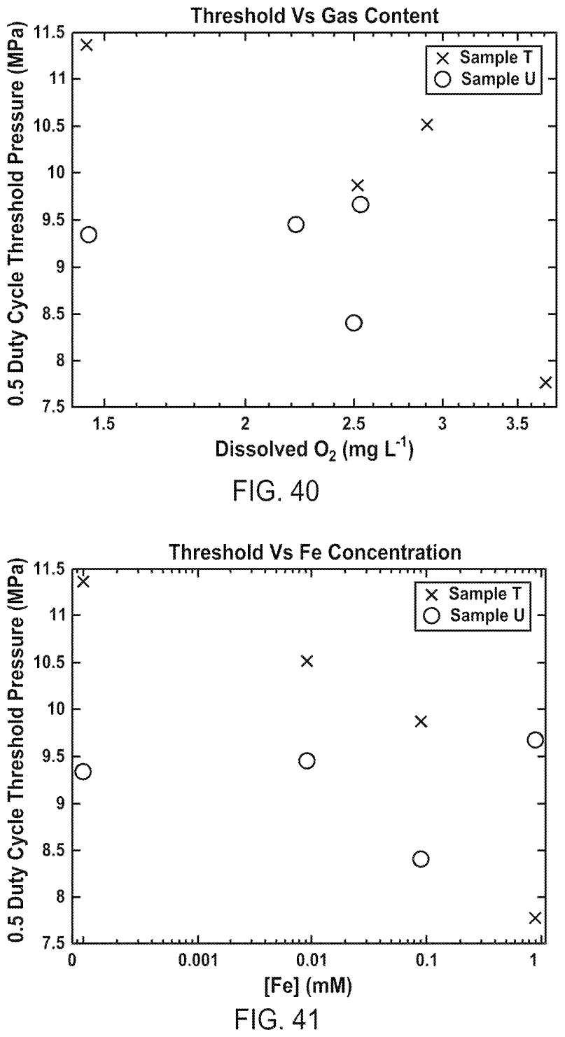

To better capture statistical variability, this set of experiments were run on two independently performed dilutions: one labeled “T” and one labeled “U”. The resulting sigmoidal curves are shown in FIG. 38 and FIG. 39, respectively. Save for one important outlier (the 0.9 mM Fe sample from source “T”), the curves represent very similar 0.5 cavitation duty cycle threshold pressures. In the case of the 0.9 mM sample from source “T,” experimental error allowed some dissolved oxygen (and likely other gases) to enter the solution prior to testing. Combined with the more gradual slopes observed for some samples derived from source “U,” it is unclear if the cavitation behavior is being determined by the concentration of nanoparticles in solution or gas content or both. To further clarify this point, the 0.5 cavitation duty cycle threshold pressures were plotted as both a function iron concentration and measured dissolved oxygen content, as shown in FIG. 40 and FIG. 41.

As can be seen in FIG. 40 and FIG. 41, the threshold appears to decrease slightly with particle concentration and gas content. However, it is still unclear which of the differences in the thresholds displayed in these figures are statistically significant and the exact relationship is difficult to determine from the data at hand. However, this behavior is very different from that observed using the commercially available particles from Example 1. For those particles, the threshold pressure (as measured using high duty cycle insonations) decreased monotonically with acoustic pressure as displayed FIG. 24.

Summary

To summarize, nanoparticles that match the desired size and surface chemistry were successfully created. When suspended the water, the particles appear to have, at best, weak influences on relevant cavitation thresholds. However, the particles displayed a lower relaxivity.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A coupling bath for use in a magnetic resonance imaging (MRI)-guided procedure in which focused energy is applied to an area of interest of a subject, the coupling bath comprising:
an aqueous solution comprising a plurality of paramagnetic particles dispersed in water,
each of the plurality of paramagnetic particles having a composition, an average particle size, a shape, a concentration, and an optional capping layer,
the optional capping layer, when present, comprising a plurality of ligands attached to a surface of the paramagnetic particle and having an average thickness,
wherein, when magnetic resonance images are collected from the area of interest of the subject for the MRI guidance:
the coupling bath is located proximate to the area of interest, and
the composition, the average particle size, the shape, the concentration, the presence or absence of the capping layer, the identity of the plurality of ligands when the capping layer is present, the average thickness of the capping layer when the capping layer is present, or a combination thereof is/are selected such that the coupling bath reduces or prevents imaging artifacts in the magnetic resonance images for the MRI guidance.

2. The coupling bath of claim 1, wherein the plurality of paramagnetic particles are biocompatible.

3. The coupling bath of claim 1, wherein the plurality of paramagnetic particles comprise a metal selected from the group consisting of Fe, Mn, Ni, Gd, and combinations thereof.

4. The coupling bath of claim 1, wherein the plurality of paramagnetic particles comprise Fe.

5. The coupling bath of claim 1, wherein the plurality of paramagnetic particles comprise an iron oxide.

6. The coupling bath of claim 1, wherein the plurality of paramagnetic particles comprise Fe₃O₄.

7. The coupling bath of claim 1, wherein the plurality of paramagnetic particles have an average particle size of 250 nanometers (nm) or less.

8. The coupling bath of claim 1, wherein the plurality of paramagnetic particles have an average particle size of from 30 nm to 50 nm.

9. The coupling bath of claim 1, wherein the plurality of paramagnetic particles are substantially spherical in shape.

10. The coupling bath of claim 1, wherein each of the plurality of paramagnetic particles further comprises a capping layer comprising a plurality of ligands, the plurality of ligands being attached to a surface of each of the plurality of paramagnetic particles.

11. The coupling bath of claim 1, wherein the plurality of ligands are hydrophilic such that the capping layer is hydrophilic.

12. The coupling bath of claim 1, wherein the plurality of ligands comprise poly((meth)acrylic acid).

13. The coupling bath of claim 1, wherein the capping layer has an average thickness of from 1 nm to 10 nm.

14. The coupling bath of claim 1, wherein the plurality of paramagnetic particles have a concentration of 10 mM or less in the coupling bath.

15-22. (canceled)

23. A method for a magnetic resonance imaging (MRI)-guided procedure on a subject wherein focused energy is applied to an area of interest of the subject, the method comprising providing the coupling bath of claim 1 proximate the area of interest, such that, when magnetic resonance images are collected from the area of interest of the subject for the MRI guidance, the coupling bath reduces or prevents imaging artifacts in the magnetic resonance images for the MRI guidance.

24-33. (canceled)

34. A system for a magnetic resonance imaging (MRI)-guided procedure on a subject wherein focused energy is applied to an area of interest of the subject, the system comprising the coupling bath of claim 1 proximate the area of interest and a magnetic resonance imaging device configured to collect images from the area of interest of the subject for the MRI guidance, wherein when the magnetic resonance images are collected from the area of interest of the subject for MRI guidance, the coupling bath reduces or prevents imaging artifacts in the magnetic resonance images for the MRI guidance.

35. The system of claim 34, further comprising a device configured to apply focused energy to the area of interest in the subject.

36. The system of claim 35, wherein the device configured to apply focused energy to the area of interest in the subject comprises a transcranial focused ultrasound (T-FUS) device.

37. The system of claim 36, wherein the T-FUS device comprises a transducer and the coupling bath is located between the transducer and the area of interest of the subject.

38. The system of claim 34, wherein the system further comprises a means for circulating the coupling bath.
